(19) United States
(12) Patent Application Publication
(10) Pub. No.: US 2026/0115213 A1
(43) Pub. Date: Apr. 30, 2026

(54) SYNTHETIC ROCAGLATES WITH BROAD-SPECTRUM ANTIVIRAL ACTIVITIES AND USES THEREOF

(71) Applicants: Memorial Sloan Kettering Cancer Center, NEW YORK, NY (US); Philipps-Universität Marburg, Marbur (DE)

(72) Inventors: HANS-GUIDO WENDEL, New York, NY (US); ARNOLD GRUNWELLER, Marburg (DE)

(73) Assignees: Memorial Sloan Kettering Cancer Center, NEW YORK, NY (US); Philipps-Universität Marburg, Marbur (DE)

(21) Appl. No.: 18/555,484

(22) PCT Filed: Apr. 14, 2022
(86) PCT No.: PCT/US2022/024790

§ 371 Date: Oct. 13, 2023

Related U.S. Application Data
(60) Provisional application No. 63/175,014, filed on Apr. 14, 2021.

(51) Int. Cl. A61K 31/665; A61K 31/343; A61P 31/14

(52) U.S. Cl. CPC A61K 31/665; A61K 31/343; A61P 31/14

(57) ABSTRACT

Described herein are compositions, uses thereof, and methods for treating a viral infection in a host cell or organism infected by the virus, such as coronaviruses, Zika virus, Lassa virus, Crimean Congo hemorrhagic fever virus, hepatitis E virus, and other RNA viruses. Also described herein are synthetic rocaglate compositions, uses thereof, and methods for reducing or inhibiting translation initiation of a messenger ribonucleic acid (mRNA) of a virus in a host cell or organism infected by the virus.

### CROSS-REFERENCE TO RELATED APPLICATIONS

This application claims benefit of U.S. Provisional Patent Application No. 63/175,014, filed Apr. 14, 2021, which is incorporated by reference herein in its entirety.

### FIELD OF INTEREST

This disclosure relates to synthetic rocaglate compositions, uses thereof, and methods for treating a viral infection in a host cell or organism infected by the virus, such as coronaviruses, Zika virus, Lassa virus, Crimean Congo hemorrhagic fever virus, and hepatitis E virus, and other RNA viruses. Also disclosed are synthetic rocaglate compositions, uses thereof, and methods for reducing or inhibiting translation initiation of a messenger ribonucleic acid (mRNA) of a virus in a host cell or organism infected by the virus.

### BACKGROUND

Rocaglates, a class of natural compounds isolated from plants of the genus Aglaia in the mahogany family (Meliaceae), are potent inhibitors of translation initiation. They are proposed to form stable stacking interactions with polypurine sequences in the 5′-UTR of selected mRNAs thereby clamping the RNA substrate onto eIF4A causing the inhibition of the translation initiation complex. The DEAD-box RNA helicase eIF4A, which is part of the heterotrimeric translation initiation complex eIF4F, unwinds RNA secondary structures in 5′-untranslated regions (5′-UTRs) of selected mRNAs to enable binding of the 43S preinitiation complex (PIC). In cells, eIF4A has a critical role in the translation of protooncogenic mRNAs with complex structured 5′-UTRs. Viral RNAs also contain highly structured 5′-UTRs, suggesting that viral protein synthesis may also be eIF4A-dependent.

The activation of protein translation contributes to malignant transformation. For example, activation of the RAS, ERK, and AKT signaling pathways stimulates cap-dependent translation. Moreover, the rate limiting eIF4E translation factor is expressed at high levels in many cancers and can transform rodent fibroblasts and promote tumor development in vivo. Accordingly, cap-dependent translation is an emerging target for cancer therapies. Notably, three distinct natural compounds target the eIF4A helicase and these are the rocaglate Silvestrol (methyl (1R,2R,3S,3aR,8bS)-6-[[(2S,3R,6R)-6-[(1R)-1,2-dihydroxyethyl]-3-methoxy-1,4-dioxan-2-yl]oxy]-1,8b-dihydroxy-8-methoxy-3a-(4-methoxyphenyl)-3-phenyl-2,3-dihydro-1H-cyclopenta[b][1]benzofuran-2-carboxylate) and at least some other natural rocaglates isolated from plants of the genus Algaia in the mahogany family (Meliaceae) in the Malaysian rainforest, the macrolide pateamine A ((3S,6Z,8E,11S,15R,17S)-15-amino-3-[(1E,3E,5E)-7-(dimethylamino)-2, 5-dimethylhepta-1,3,5-trienyl]-9,11,17-trimethyl-4, 12-dioxa-20-thia-21-azabicyclo[16.2.1]henicosa-1(21),6,8,18-tetraene-5, 13-dione)) found in marine sponges off the coast of New Zealand, and the steroid hippuristanol which is produced by Pacific corals. These compounds show promising preclinical activity against different cancers. Other strategies to inhibit translation include rapamycin and mTORC1 kinase inhibitors, inhibitors of the eIF4E kinase MNK1/2, a peptide (4EGI-1) that interferes with the eIF4E-eIF4G interaction, and the anti-viral ribavirin that may bind eIF4E directly.

Rocaglates are members of a super family of natural products incorporating a common cyclopentyl[b]furan core. Many members of this family, including Silvestrol, are potent inhibitors of translation initiation and exhibit single-agent, antineoplastic activity in preclinical assays (both in vitro and in vivo). A significant body of evidence suggests that these agents are inhibitors of eIF4A RNA helicase. They function by preventing translation initiation by hindering helicase unwinding via eIF4A inhibition and interfering with ribosome recruitment to mRNA templates. Briefly, eIF4A is selectively required for the translation of mRNAs with G-quadruplex (GQ) structures in their 5′UTRs. These ˜220 GQ mRNAs include oncogenes such as c-MYC, N-Myc, L-Myc, N-RAS, MYB, Notch1, BCL2, and CDK6.

In this regard, the specific eIF4A inhibitor Silvestrol, a plant-derived rocaglate, has broad-spectrum antiviral activity at non-cytotoxic concentrations in a low nanomolar range. Silvestrol inhibits the replication of RNA viruses representing different virus families, like Ebola- (EBOV), Corona- (CoV), Zika- (ZIKV), Chikungunya- (CHIKV), and hepatitis E (HEV) viruses. Notably, Silvestrol showed good bioavailability, in vivo antiviral activity and low cytotoxicity in primary cells. However, synthesis of Silvestrol is sophisticated, difficult, and time-consuming, thus hampering its prospects for further antiviral clinical development.

It would be desirable to have alternative strategies utilizing additional compositions and methods for inhibiting the replication of RNA viruses, including coronaviruses. It would also be desirable to have compositions and methods for treating or preventing human and other animal infections by RNA viruses, including coronaviruses.

### SUMMARY

The compositions and methods provided herein are directed to inhibiting the replication of RNA viruses, including coronaviruses, and to treating or preventing human or other animal infections by RNA viruses, including coronaviruses.

Disclosed herein are methods of treating a viral infection in a host cell or organism infected by the virus, the methods comprising administering to the cell or organism a therapeutically effective amount of a pharmaceutical composition comprising a rocaglate compound or a pharmaceutically acceptable salt thereof, method of treating a viral infection in a host cell or organism infected by the virus, the method comprising administering to the cell or organism a therapeutically effective amount of a pharmaceutical composition comprising a rocaglate compound or a pharmaceutically acceptable salt thereof, wherein the rocaglate compound comprises a compound represented by formula (I)

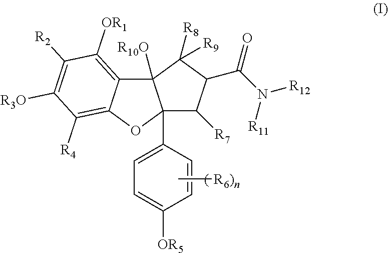

wherein R1, R3, and R5 are each independently H, alkyl, —P(O)(OH)(OH), —CH2-P(O)(OH)(OH), —P(O)(OH)(O-alkyl), —CH2-P(O)(OH)(O-alkyl), —P(O)(O-alkyl)(O-alkyl), —CH2-P(O)(O-alkyl)(O-alkyl), or (CO)-alkyl, or a pharmaceutically acceptable salt of —P(O)(OH)(OH), —CH2-P(O)(OH)(OH), —P(O)(OH)(O-alkyl), or —CH2-P(O)(OH)(O-alkyl), wherein at least one of R1, R3, and R5 is H, —P(O)(OH)(OH), —CH2-P(O)(OH)(OH), —P(O)(OH)(O-alkyl), —CH2-P(O)(OH)(O-alkyl), —P(O)(O-alkyl)(O-alkyl), —CH2-P(O)(O-alkyl)(O-alkyl), or a pharmaceutically acceptable salt of —P(O)(OH)(OH), —CH2-P(O)(OH)(OH), —P(O)(OH)(O-alkyl), or —CH2-P(O)(OH)(O-alkyl); R2 and R4 are each independently H, alkyl, halo, nitro, OH, O-alkyl, SH, S-alkyl, CN, haloalkyl, O-haloalkyl, NRaRb, (CO)-alkyl, (CO)OH, (CO)O-alkyl, SO2NRaRb, (CO)NRaRb, NH(CO)-alkyl, cycloalkyl, heterocycloalkyl, aryl, or heteroaryl, wherein aryl and heteroaryl are optionally substituted with alkyl, halo, nitro, OH, O-alkyl, SH, S-alkyl, CN, haloalkyl, O-haloalkyl, and NRaRb; R6 is alkyl, halo, nitro, OH, O-alkyl, SH, S-alkyl, CN, haloalkyl, O-haloalkyl, NRaRb, (CO)-alkyl, (CO)OH, (CO)O-alkyl, SO2NRaRb, (CO)NRaRb, NH(CO)-alkyl, cycloalkyl, heterocycloalkyl, aryl, or heteroaryl, wherein aryl and heteroaryl are optionally substituted with alkyl, halo, nitro, OH, O-alkyl, SH, S-alkyl, CN, haloalkyl, O-haloalkyl, and NRaRb; R7 is aryl or heteroaryl, wherein aryl and heteroaryl are optionally substituted with alkyl, halo, nitro, OH, O-alkyl, SH, S-alkyl, CN, haloalkyl, O-haloalkyl, and NRaRb; R8 and R9 are each independently H, OH, alkyl, halo, O-alkyl, SH, S-alkyl, CN, haloalkyl, O-haloalkyl, NRaRb, (CO)OH, (CO)O-alkyl, SO2NRaRb, (CO)NRaRb, or NH(CO)-alkyl; R10 is H, alkyl, (CO)-alkyl, or (CO)NRaRb; R11 and R12 are each independently H, OH, alkyloxy, cycloalkyloxy, heterocycloalkyloxy, cycloalkylalkyloxy, heterocycloalkylalkyloxy, arylalkyloxy, heteroarylalkyloxy, aryloxy, or heteroaryloxy; Ra and Rb are each H or alkyl, or Ra and Rb, together with the nitrogen atom they are attached, form a heterocycloalkyl group; and n is an integer from 0 to 4, or a pharmaceutically acceptable salt thereof.

Also disclosed herein are methods for reducing or inhibiting translation initiation of a messenger ribonucleic acid (mRNA) of a virus in a host cell or organism infected by the virus, the methods comprising administering to the cell or organism a therapeutically effective amount of a pharmaceutical composition comprising a rocaglate compound of formula (I) or a pharmaceutically acceptable salt thereof.

Also disclosed herein are uses of a synthetic rocaglate composition for reducing or inhibiting translation initiation of a messenger ribonucleic acid (mRNA) of a virus in a host cell or organism infected by the virus, the synthetic rocaglate composition comprising a therapeutically effective amount of a rocaglate composition of formula (I) or a pharmaceutically acceptable salt thereof.

For any of the foregoing uses, the synthetic rocaglate composition comprises

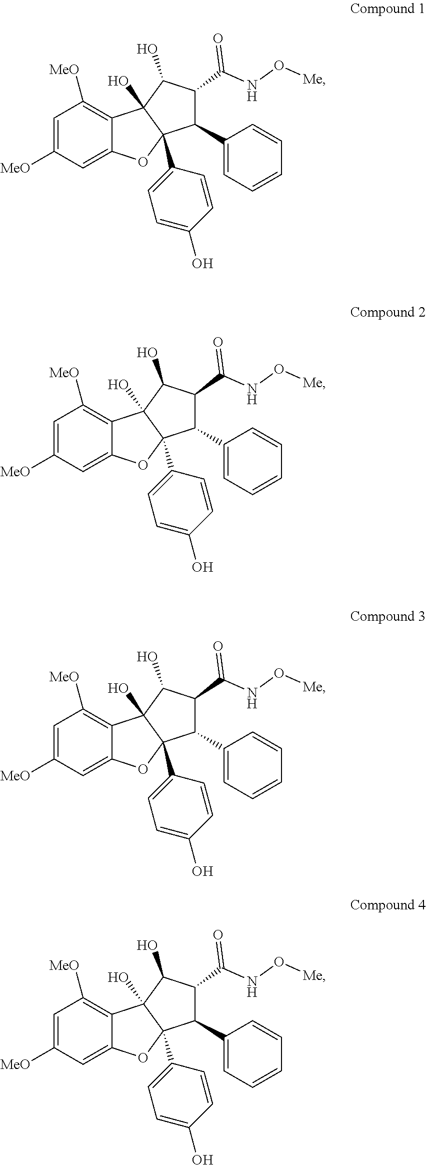 [SMILES: CONC(=O)[C@H]1[C@@H](O)[C@@]2(O)c3c(OC)cc(OC)cc3O[C@@]2(c2ccc(O)cc2)[C@@H]1c1ccccc1 ; CONC(=O)[C@@H]1[C@H](O)[C@]2(O)c3c(OC)cc(OC)cc3O[C@]2(c2ccc(O)cc2)[C@H]1c1ccccc1 ; CONC(=O)[C@@H]1[C@@H](O)[C@@]2(O)c3c(OC)cc(OC)cc3O[C@@]2(c2ccc(O)cc2)[C@H]1c1ccccc1 ; CONC(=O)[C@@H]1[C@@H](c2ccccc2)[C@@]2(c3ccc(O)cc3)Oc3cc(OC)cc(OC)c3[C@@]2(O)[C@H]1O]

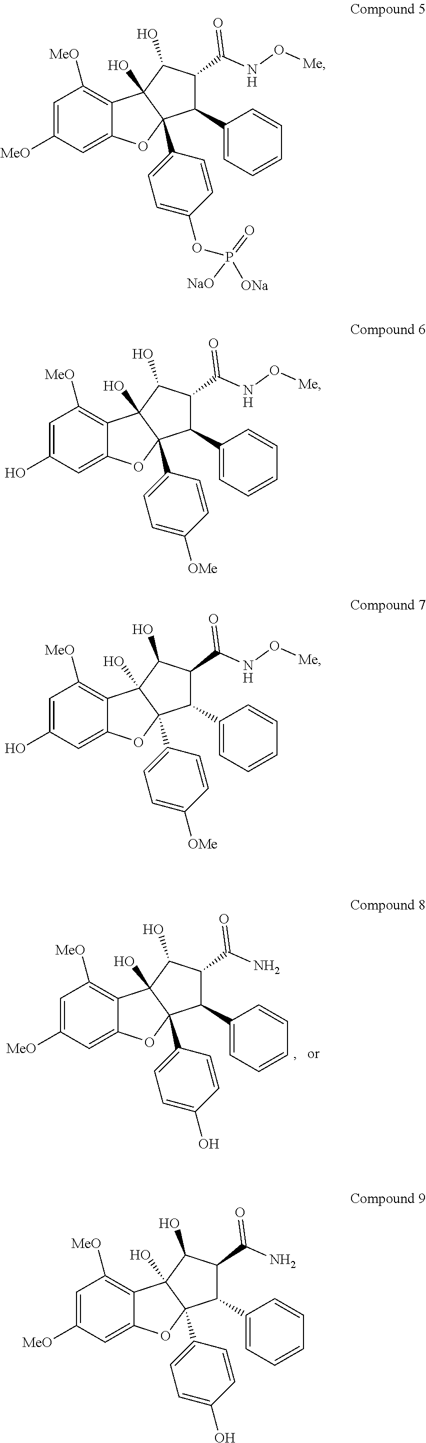

or an enantiomer of any thereof, a racemic mixture of any thereof, or a combination of any thereof.

In some embodiments, the synthetic rocaglate composition comprises Compound 9, a racemic mixture of Compound 9 and Compound 8, Compound 8, Compound 6, a racemic mixture of Compound 6 and Compound 7, Compound 1, Compound 5, or Compound 7.

In some embodiments, the synthetic rocaglate composition comprises at least 50% Compound 9 enantiomer. In some embodiments, the synthetic rocaglate composition is Compound 9 enantiomer.

In some embodiments, the synthetic rocaglate composition reduces or inhibits a eukaryotic initiation factor 4A (eIF4A) activity. In some embodiments, the synthetic rocaglate composition reduces or inhibits the eIF4A helicase activity. In some embodiments, the synthetic rocaglate composition reduces or inhibits eIF4A clamping to a 5′-untranslated region (5′-UTR) of the mRNA of the virus. In some embodiments, the 5′-UTR comprises a hairpin structure. In some embodiments, the 5′-UTR comprises a polypurine sequence element comprising at least 10 purine nucleotides. In some embodiments, the polypurine sequence element comprises at least 20 or at least 30 purine nucleotides.

In some embodiments, the virus is an RNA virus. In some embodiments, the virus comprises a virus from the Coronaviridae family, the Arenaviridae family, the Nairoviridae family, the Flaviviridae family, the Hepeviridae family, the Filoviridae family, or the Togaviridae family. In some embodiments, the virus from the Coronaviridae family comprises human coronavirus 229E (HCoV-229E), Middle East respiratory syndrome coronavirus (MERS-CoV), severe acute respiratory syndrome coronavirus (SARS-CoV), severe acute respiratory syndrome coronavirus 2 (SARS-CoV-2, COVID-19 virus), human coronavirus OC43 (HCoV-OC43), human coronavirus NL63 (HCoV-NL63), or human coronavirus HKU1 (HCoV-HKU1). In some embodiments, the virus from the Arenaviridae family comprises Lassa mammarenavirus (LASV), Guanarito mammarenavirus, Junin mammarenavirus, Lujo mammarenavirus, Machupo mammarenavirus, Sabia mammarenavirus, or Whitewater Arroyo mammarenavirus. In some embodiments, the virus from the Nairoviridae family comprises Crimean-Congo hemorrhagic fever virus (CCHFV). In some embodiments, the virus from the Flaviviridae family comprises Zika virus (ZIKV), hepacivirus C (hepatitis C virus, HepC), dengue fever virus, yellow fever virus, Japanese encephalitis virus, or West Nile virus. In some embodiments, the virus from the Hepeviridae family comprises hepatitis E virus (HEV) or hepatitis B virus.

The method of claim 12, wherein the virus from the Filoviridae family comprises Ebolavirus, Marburgvirus, Dianlovirus, Cuevavirus, Striavirus, or Thamnovirus. In some embodiments, the virus from the Togaviridae family comprises an Alphavirus. In some embodiments, the virus from the Alphavirus comprises Chikungunya virus, Eastern equine encephalitis virus, Western equine encephalitis virus, Barmah Forest virus, Mayaro virus, O'nyong'nyong virus, Ross river virus, Semliki Forest virus, Sindbis virus, Una virus, Tonate virus, or Venezuelan equine encephalitis.

In some embodiments, the pharmaceutical composition further comprises a pharmaceutically acceptable carrier, excipient, or diluent.

In another aspect, use of a synthetic rocaglate composition for treating a viral infection in a host cell or organism infected by a virus, the synthetic rocaglate composition comprising a therapeutically effective amount of a compound represented by formula (I)

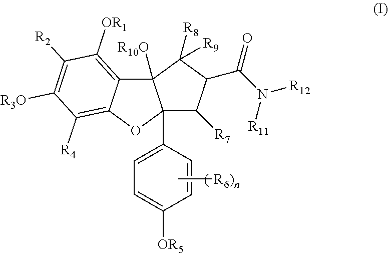

wherein
R1, R3, and R5 are each independently H, alkyl, —P(O)(OH)(OH), —CH2-P(O)(OH)(OH), —P(O)(OH)(O-alkyl), —CH2-P(O)(OH)(O-alkyl), —P(O)(O-alkyl)(O-alkyl), —CH2-P(O)(O-alkyl)(O-alkyl), or (CO)-alkyl, or a pharmaceutically acceptable salt of —P(O)(OH)(OH), —CH2-P(O)(OH)(OH), —P(O)(OH)(O-alkyl), or —CH2-P(O)(OH)(O-alkyl), wherein at least one of R1, R3, and R5 is H, —P(O)(OH)(OH), —CH2-P(O)(OH)(OH), —P(O)(OH)(O-alkyl), —CH2-P(O)(OH)(O-alkyl), —P(O)(O-alkyl)(O-alkyl), —CH2-P(O)(O-alkyl)(O-alkyl), or a pharmaceutically acceptable salt of —P(O)(OH)(OH), —CH2-P(O)(OH)(OH), —P(O)(OH)(O-alkyl), or —CH2-P(O)(OH)(O-alkyl);
R2 and R4 are each independently H, alkyl, halo, nitro, OH, O-alkyl, SH, S-alkyl, CN, haloalkyl, O-haloalkyl, NRaRb, (CO)-alkyl, (CO)OH, (CO)O-alkyl, SO2NRaRb, (CO)NRaRb, NH(CO)-alkyl, cycloalkyl, heterocycloalkyl, aryl, or heteroaryl, wherein aryl and heteroaryl are optionally substituted with alkyl, halo, nitro, OH, O-alkyl, SH, S-alkyl, CN, haloalkyl, O-haloalkyl, and NRaRb;
R6 is alkyl, halo, nitro, OH, O-alkyl, SH, S-alkyl, CN, haloalkyl, O-haloalkyl, NRaRb, (CO)-alkyl, (CO)OH, (CO)O-alkyl, SO2NRaRb, (CO)NRaRb, NH(CO)-alkyl, cycloalkyl, heterocycloalkyl, aryl, or heteroaryl, wherein aryl and heteroaryl are optionally substituted with alkyl, halo, nitro, OH, O-alkyl, SH, S-alkyl, CN, haloalkyl, O-haloalkyl, and NRaRb;
R7 is aryl or heteroaryl, wherein aryl and heteroaryl are optionally substituted with alkyl, halo, nitro, OH, O-alkyl, SH, S-alkyl, CN, haloalkyl, O-haloalkyl, and NRaRb;
R8 and R9 are each independently H, OH, alkyl, halo, O-alkyl, SH, S-alkyl, CN, haloalkyl, O-haloalkyl, NRaRb, (CO)OH, (CO)O-alkyl, SO2NRaRb, (CO)NRaRb, or NH(CO)-alkyl;
R10 is H, alkyl, (CO)-alkyl, or (CO)NRaRb;
R11 and R12 are each independently H, OH, alkyloxy, cycloalkyloxy, heterocycloalkyloxy, cycloalkylalkyloxy, heterocycloalkylalkyloxy, arylalkyloxy, heteroarylalkyloxy, aryloxy, or heteroaryloxy;
Ra and Rb are each H or alkyl, or Ra and Rb, together with the nitrogen atom they are attached, form a heterocycloalkyl group; and
n is an integer from 0 to 4,
or a pharmaceutically acceptable salt thereof.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic depicting a comparison between the structures of various rocaglates. FIG. 1 depicts the structures of Compound 1 (R=H; (1R,2R,3S,3aR,8bS)-1,8b-dihydroxy-3a-(4-hydroxyphenyl)-N,6,8-trimethoxy-3-phenyl-2,3-dihydro-1Hcyclopenta[b]benzofuran-2-carboxamide) and Compound 5 (R=PO₃²; sodium 4-((1R,2R,3S,3aR,8bS)-1,8b-dihydroxy-6,8-dimethoxy-2-(methoxycarbamoyl)-3-phenyl-2,3,3a,8b-tetrahydro-1H-cyclopenta[b]benzofuran-3a-yl)phenyl phosphate) (upper left); Compounds 8+9 (racemic mixture of (1S,2S,3R,3aS,8bR)-1,8b-dihydroxy-3a-(4-hydroxyphenyl)-6,8-dimethoxy-3-phenyl-2,3-dihydro-1H-cyclopenta[b]benzofuran-2-carboxamide [Compound 8] and (1R,2R,3S,3aR,8bS)-1,8b-dihydroxy-3a-(4-hydroxyphenyl)-6,8-dimethoxy-3-phenyl-2,3-dihydro-1H-cyclopenta[b]benzofuran-2-carboxamide [Compound 9]), Compound 8, and Compound 9 (upper right); and Compound 6 ((1R,2R,3S,3aR,8bS)-1,6,8b-trihydroxy-N,8-dimethoxy-3a-(4-methoxyphenyl)-3-phenyl-2,3-dihydro-1Hcyclopenta[b]benzofuran-2-carboxamide) and Compound 7 ((1S,2S,3R,3aS,8bR)-1,6,8b-trihydroxy-N,8-dimethoxy-3a-(4-methoxyphenyl)-3-phenyl-2,3,3a,8b-tetrahydro-1H-cyclopenta[b]benzofuran-2-carboxamide), lower left.

FIG. 2 is a schematic and a graph depicting a dual luciferase assay. FIG. 12A is a schematic of the dual luciferase assay used to analyze the sensitivity of viral 5′-untranslated regions (5′-UTRs) towards eIF4A inhibition.

FIG. 3 is a graph showing the resulting analysis of the sensitivity of the 5′-UTR of beta-globin (P-globin; shades of blue; left-to-right 5, 10, 50 and 100 nM) negative control (eIF4A-independent translation), and the sensitivity of the 5′-UTR of (AG)15 ([AG]₁₅; shades of red; left-to-right 5, 10, 50 and 100 nM) positive control (eIF5A-dependent translation), against 5, 10, 50, and 100 nM treatment with five rocaglate samples (Compound 1, Compound 6, Compound 7, Compound 8, Compound 9) in a dual luciferase assay. The reporter gene expression data were normalized to the transfection efficiencies and the corresponding DMSO controls. An eIF4A-independent effect was observed for Compound 1, while Compound 9 only reduced reporter activity with polyAG sequences. Therefore, only Compound 9 show eIF4A-dependency, while Compound 1 has nonspecific effects (B-Globin 5′-UTR). Standard errors of the mean of at least three independent experiments are shown. MFE=minimal free energy (kcal/mol). For each compound, bars from left to right are: 5, 10, 50 and 100 nM compound on 5′-UTR of beta-globin (β-globin) negative control (eIF4A-independent translation), and 5, 10, 50 and 100 nM compound on 5′-UTR of (AG)₁₅ positive control (eIF5A-dependent translation).

FIG. 4 is a graph (left) and a table (right) depicting the melting temperature (Tm; Tm) of eIF4A1(19-406)-(AG)5-AMPPNP-Rocaglate/DMSO complexes, using DMSO as a negative control, as indicative of eIF4A binding of various rocaglate compounds (Compound 1, Compound 6, Compound 7, Compound 8, Compound 9) described herein (eIF4A1(19-406)=amino acid residues 19-406 of eIF4A1); AMPPNP=adenylyl-imidodiphosphate [AMP-PNP]).

FIG. 5 is a graph depicting the comparative effects of Compound 8 and Compound 9 vs. Untreated cells in Normal Human Bronchial Epithelial cells (NHBE cells), as a measure of virus titer over time, from a donor infected with SARS-CoV-2 (COVID-19 virus). The cells of the donor were treated with 50 nM or 500 nM Compound 9 as compared with untreated cells or with cells treated with 500 nM Compound 8 as controls.

FIG. 6 is an immunofluorescence analysis to determine the effects of Compound 8 and 9 on viral dsRNA accumulation in SARS-CoV-2 infected Vero E6 cells. After infection and treatment with Compound 8 and 9 cells were fixed at 24 h p.i. and analyzed by confocal microscopy using a mouse anti-dsRNA mAB (J2, SCICONS English & Scientific Consulting Kft) that detects a viral double-stranded (ds) RNA replication intermediate (red) (Müller C, Hardt M, Schwudke D, Neuman B W, Pleschka S, Ziebuhr J. 2018. Inhibition of cytosolic phospholipase A2a impairs an early step of coronavirus replication in cell culture. J Virol 92:e01463-17). Cell nuclei were stained with 2-(4-amidinophenyl)-1H-indole-6-carboxamidine (4′,6-diamidino-2-phenylindole; DAPI; blue). Samples were treated as follows: untreated control, mock infection control, Compound 8 (500 nM), Compound 9 (50 nM), Compound 9 (500 nM) (left to right).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

It would be desirable to synthesize rocaglates having antiviral properties.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Disclosed herein are methods of treating a viral infection in a host cell or organism infected by the virus, the methods comprising administering to the cell or organism a therapeutically effective amount of a pharmaceutical composition comprising a rocaglate compound or a pharmaceutically acceptable salt thereof, method of treating a viral infection in a host cell or organism infected by the virus, the method comprising administering to the cell or organism a therapeutically effective amount of a pharmaceutical composition comprising a rocaglate compound or a pharmaceutically acceptable salt thereof, wherein the rocaglate compound comprises a compound represented by formula (I)

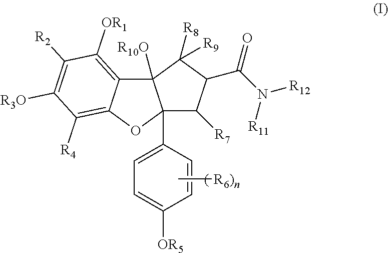

wherein R1, R3, and R5 are each independently H, alkyl, —P(O)(OH)(OH), —CH2-P(O)(OH)(OH), —P(O)(OH)(O-alkyl), —CH2-P(O)(OH)(O-alkyl), —P(O)(O-alkyl)(O-alkyl), —CH2-P(O)(O-alkyl)(O-alkyl), or (CO)-alkyl, or a pharmaceutically acceptable salt of —P(O)(OH)(OH), —CH2-P(O)(OH)(OH), —P(O)(OH)(O-alkyl), or —CH2-P(O)(OH)(O-alkyl), wherein at least one of R1, R3, and R5 is H, —P(O)(OH)(OH), —CH2-P(O)(OH)(OH), —P(O)(OH)(O-alkyl), —CH2-P(O)(OH)(O-alkyl), —P(O)(O-alkyl)(O-alkyl), —CH2-P(O)(O-alkyl)(O-alkyl), or a pharmaceutically acceptable salt of —P(O)(OH)(OH), —CH2-P(O)(OH)(OH), —P(O)(OH)(O-alkyl), or —CH2-P(O)(OH)(O-alkyl); R2 and R4 are each independently H, alkyl, halo, nitro, OH, O-alkyl, SH, S-alkyl, CN, haloalkyl, O-haloalkyl, NRaRb, (CO)-alkyl, (CO)OH, (CO)O-alkyl, SO2NRaRb, (CO)NRaRb, NH(CO)-alkyl, cycloalkyl, heterocycloalkyl, aryl, or heteroaryl, wherein aryl and heteroaryl are optionally substituted with alkyl, halo, nitro, OH, O-alkyl, SH, S-alkyl, CN, haloalkyl, O-haloalkyl, and NRaRb; R6 is alkyl, halo, nitro, OH, O-alkyl, SH, S-alkyl, CN, haloalkyl, O-haloalkyl, NRaRb, (CO)-alkyl, (CO)OH, (CO)O-alkyl, SO2NRaRb, (CO)NRaRb, NH(CO)-alkyl, cycloalkyl, heterocycloalkyl, aryl, or heteroaryl, wherein aryl and heteroaryl are optionally substituted with alkyl, halo, nitro, OH, O-alkyl, SH, S-alkyl, CN, haloalkyl, O-haloalkyl, and NRaRb; R7 is aryl or heteroaryl, wherein aryl and heteroaryl are optionally substituted with alkyl, halo, nitro, OH, O-alkyl, SH, S-alkyl, CN, haloalkyl, O-haloalkyl, and NRaRb; R8 and R9 are each independently H, OH, alkyl, halo, O-alkyl, SH, S-alkyl, CN, haloalkyl, O-haloalkyl, NRaRb, (CO)OH, (CO)O-alkyl, SO2NRaRb, (CO)NRaRb, or NH(CO)-alkyl; R10 is H, alkyl, (CO)-alkyl, or (CO)NRaRb; R11 and R12 are each independently H, OH, alkyloxy, cycloalkyloxy, heterocycloalkyloxy, cycloalkylalkyloxy, heterocycloalkylalkyloxy, arylalkyloxy, heteroarylalkyloxy, aryloxy, or heteroaryloxy; Ra and Rb are each H or alkyl, or Ra and Rb, together with the nitrogen atom they are attached, form a heterocycloalkyl group; and n is an integer from 0 to 4, or a pharmaceutically acceptable salt thereof.

In some embodiments, R11 is H. In some embodiments, R₁₁ and R₁₂ both are H. In some embodiments, R₁₂ is alkyloxy or cycloalkyloxy. In some embodiments, R₁₂ is alkyloxy. In certain embodiments, R₁₂ is OMe. In some embodiments, R₁₁ is H and R₁₂ is alkyloxy or cycloalkyloxy. In other embodiments, Rn is H and R₁₂ is OMe.

In some embodiments, R₃ is H, —P(O)(OH)(OH), —CH₂—P(O)(OH)(OH), —P(O)(OH)(O-alkyl), —CH₂—P(O)(OH)(O-alkyl), —P(O)(O-alkyl)(O-alkyl), —CH₂—P(O)(O-alkyl)(O-alkyl), or a pharmaceutically acceptable salt of —P(O)(OH)(OH), —CH₂—P(O)(OH)(OH), —P(O)(OH)(O-alkyl), or —CH₂—P(O)(OH)(O-alkyl). In some embodiments, R₃ is H. In other embodiments, R₃ is —P(O)(OH)(OH), —CH₂—P(O)(OH)(OH), —P(O)(OH)(O-alkyl), —CH₂—P(O)(OH)(O-alkyl), —P(O)(O-alkyl)(O-alkyl), —CH₂—P(O)(O-alkyl)(O-alkyl), or a pharmaceutically acceptable salt of —P(O)(OH)(OH), —CH₂—P(O)(OH)(OH), —P(O)(OH)(O-alkyl), or —CH₂—P(O)(OH)(O-alkyl). In other embodiments, R₃ is —P(O)(OH)(OH), —P(O)(OH)(O-alkyl), or —P(O)(O-alkyl)(O-alkyl), or a pharmaceutically acceptable salt of —P(O)(OH)(OH) or —P(O)(OH)(O-alkyl). In some embodiments, R₃ is —P(O)(OH)(OH) or —CH₂—P(O)(OH)(OH), or a pharmaceutically acceptable salt thereof. In some embodiments, R₃ is —P(O)(ONa)(OH). In other embodiments, R₃ is —P(O)(ONa)(ONa). In certain embodiments, R₃ is —P(O)(ONa)(O-alkyl).

In some embodiments, R₅ is H, —P(O)(OH)(OH), —CH₂—P(O)(OH)(OH), —P(O)(OH)(O-alkyl), —CH₂—P(O)(OH)(O-alkyl), —P(O)(O-alkyl)(O-alkyl), —CH₂—P(O)(O-alkyl)(O-alkyl), or a pharmaceutically acceptable salt of —P(O)(OH)(OH), —CH₂—P(O)(OH)(OH), —P(O)(OH)(O-alkyl), or —CH₂—P(O)(OH)(O-alkyl). In some embodiments, R₅ is H. In other embodiments, R₅ is —P(O)(OH)(OH), —CH₂—P(O)(OH)(OH), —P(O)(OH)(O-alkyl), —CH₂—P(O)(OH)(O-alkyl), —P(O)(O-alkyl)(O-alkyl), —CH₂—P(O)(O-alkyl)(O-alkyl), or a pharmaceutically acceptable salt of —P(O)(OH)(OH), —CH₂—P(O)(OH)(OH), —P(O)(OH)(O-alkyl), or —CH₂—P(O)(OH)(O-alkyl). In other embodiments, R₅ is —P(O)(OH)(OH), —P(O)(OH)(O-alkyl), or —P(O)(O-alkyl)(O-alkyl), or a pharmaceutically acceptable salt of —P(O)(OH)(OH) or —P(O)(OH)(O-alkyl). In some embodiments, R₅ is —P(O)(OH)(OH) or —CH₂—P(O)(OH)(OH), or a pharmaceutically acceptable salt thereof. In some embodiments, R₅ is —P(O)(ONa)(OH). In other embodiments, R₅ is —P(O)(ONa)(ONa). In certain embodiments, R₅ is —P(O)(ONa)(O-alkyl).

In some embodiments, R₁ is H, —P(O)(OH)(OH), —CH₂—P(O)(OH)(OH), —P(O)(OH)(O-alkyl), —CH₂—P(O)(OH)(O-alkyl), —P(O)(O-alkyl)(O-alkyl), —CH₂—P(O)(O-alkyl)(O-alkyl), or a pharmaceutically acceptable salt of —P(O)(OH)(OH), —CH₂—P(O)(OH)(OH), —P(O)(OH)(O-alkyl), or —CH₂—P(O)(OH)(O-alkyl). In some embodiments, R₁ is H. In other embodiments, R₁ is —P(O)(OH)(OH), —CH₂—P(O)(OH)(OH), —P(O)(OH)(O-alkyl), —CH₂—P(O)(OH)(O-alkyl), —P(O)(O-alkyl)(O-alkyl), —CH₂—P(O)(O-alkyl)(O-alkyl), or a pharmaceutically acceptable salt of —P(O)(OH)(OH), —CH₂—P(O)(OH)(OH), —P(O)(OH)(O-alkyl), or —CH₂—P(O)(OH)(O-alkyl). In other embodiments, R₁ is —P(O)(OH)(OH), —P(O)(OH)(O-alkyl), or —P(O)(O-alkyl)(O-alkyl), or a pharmaceutically acceptable salt of —P(O)(OH)(OH) or —P(O)(OH)(O-alkyl). In some embodiments, R₁ is —P(O)(OH)(OH) or —CH₂—P(O)(OH)(OH), or a pharmaceutically acceptable salt thereof. In some embodiments, R₁ is —P(O)(OH)(OH). In some embodiments, the pharmaceutically acceptable salt of —P(O)(OH)(OH), —CH₂—P(O)(OH)(OH), —P(O)(OH)(O-alkyl), or —CH₂—P(O)(OH)(O-alkyl) is a sodium salt, a potassium salt, a calcium salt, or other salts known in the art. In some embodiments, R₁ is —P(O)(ONa)(OH). In other embodiments, R₁ is —P(O)(ONa)(ONa). In certain embodiments, R₁ is —P(O)(ONa)(O-alkyl). In some embodiments, R₁ is H. In other embodiments, R₁ is alkyl. In certain embodiments, R₁ is methyl.

In some embodiments, R₂ and R₄ are H. In some embodiments, n is 0. In other embodiments, n is 1. In some embodiments, n is 2, 3, or 4. In some embodiments, R2 and R₄ are H and n is 0. In some embodiments, R₁₀ is H. In some embodiments, R₈ is H and R₉ is OH.

In some embodiments, the compound of formula (I) is represented by a compound of formula (II)

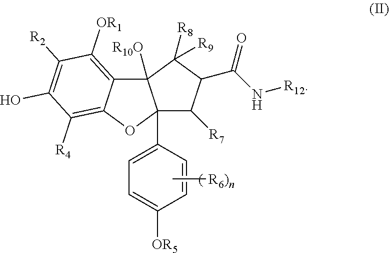

In some embodiments, in the compound of formula (II), R₁, R₂, R₄, R₅, R₆, R₇, R₈, R₉, R₁₀, R₁₂ and n are defined as anywhere herein. In some embodiments, R₁₂ is H or OMe. In some embodiments, R₁₂ is OMe. In other embodiments, R₁₂ is H.

In some embodiments, the compound of formula (I) is represented by a compound of formula (III)

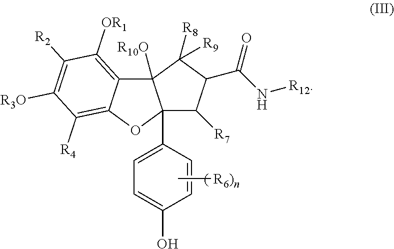

In some embodiments, in the compound of formula (III), R₁, R₂, R₃, R₄, R₆, R₇, R₈, R₉, R₁₀, R₁₂ and n are defined as anywhere herein. In some embodiments, R₁₂ is OMe. In other embodiments, R₁₂ is H.

In some embodiments, the compound of formula (I) is represented by a compound of formula (IV)

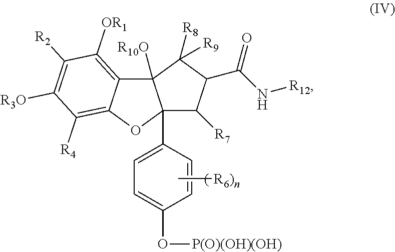

or a pharmaceutically acceptable salt thereof.

In some embodiments, in the compound of formula (IV), R₁, R₂, R₃, R₄, R₆, R₇, R₈, R₉, R₁₀, R₁₂ and n are defined as anywhere herein. In some embodiments, R₁₂ is alkyloxy or cycloalkyloxy. In some embodiments, R₁₂ is OMe.

In some embodiments, the compound of formula (I) is represented by a compound of formula (V)

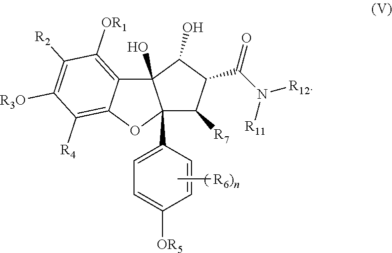

In some embodiments, the compound of formula (I) is a compound of formula (VI)

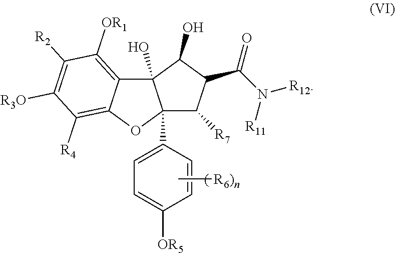

In some embodiments, the compound of formula (2) is

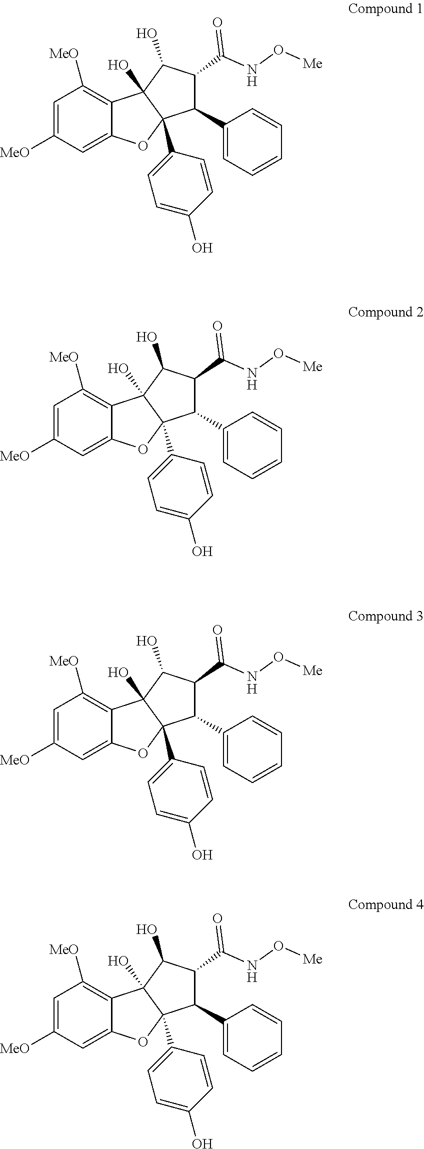 [SMILES: CONC(=O)[C@H]1[C@@H](O)[C@@]2(O)c3c(OC)cc(OC)cc3O[C@@]2(c2ccc(O)cc2)[C@@H]1c1ccccc1 ; CONC(=O)[C@@H]1[C@H](O)[C@]2(O)c3c(OC)cc(OC)cc3O[C@]2(c2ccc(O)cc2)[C@H]1c1ccccc1 ; CONC(=O)[C@@H]1[C@@H](O)[C@@]2(O)c3c(OC)cc(OC)cc3O[C@@]2(c2ccc(O)cc2)[C@H]1c1ccccc1 ; CONC(=O)[C@@H]1[C@@H](c2ccccc2)[C@@]2(c3ccc(O)cc3)Oc3cc(OC)cc(OC)c3[C@@]2(O)[C@H]1O]

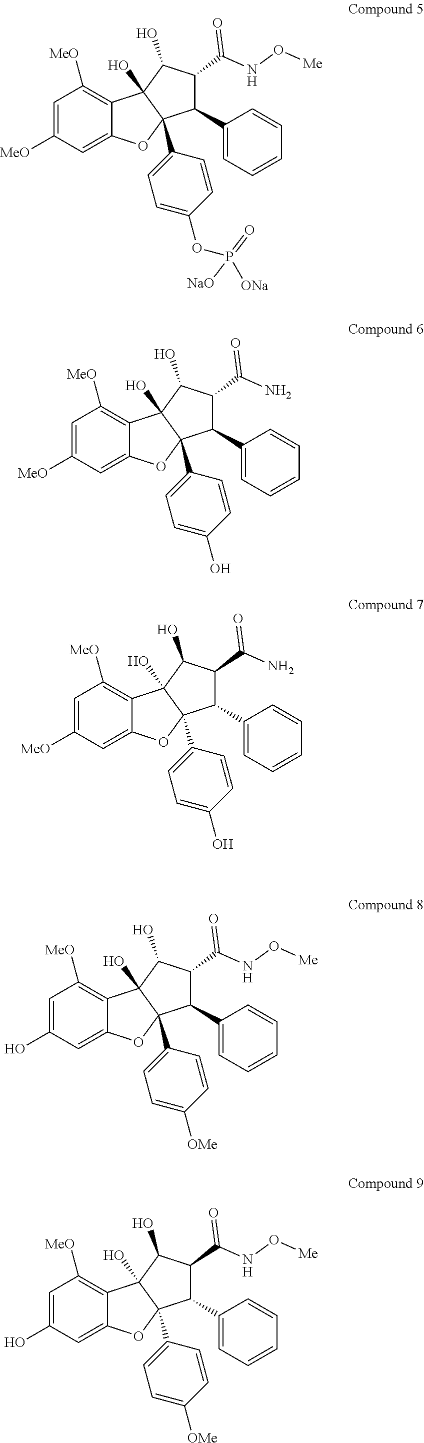

In other embodiments, the rocaglate compound is a racemic mixture comprising at least 50% Compound 9 enantiomer. In other embodiments, the rocaglate compound is Compound 9 enantiomer.

In other embodiments, the rocaglate compound is a racemic mixture comprising at least 50% Compound 6 enantiomer. In other embodiments, the rocaglate compound is Compound 6 enantiomer.

In other embodiments, the rocaglate compound is a racemic mixture comprising at least 50% Compound 1 enantiomer. In other embodiments, the rocaglate compound is Compound 1 enantiomer.

In other embodiments, the rocaglate compound is a racemic mixture comprising at least 50% Compound 5 enantiomer. In other embodiments, the rocaglate compound is Compound 5 enantiomer.

In some embodiments, the rocaglate compound reduces or inhibits a eukaryotic initiation factor 4A (eIF4A) activity. In some embodiments, the rocaglate compound reduces or inhibits an eIF4A helicase activity. In some embodiments, the rocaglate compound reduces or inhibits eIF4A clamping to a 5′-untranslated region (5′-UTR) of the mRNA of the virus. In some embodiments, the 5′-UTR comprises a hairpin structure. In some embodiments, the 5′-UTR comprises a polypurine sequence element comprising at least 10 purine nucleotides. In some embodiments, the 5′-UTR comprises a polypurine sequence element comprising at least 20 purine nucleotides. In some embodiments, the polypurine sequence element comprises at least 30 purine nucleotides.

In some embodiments, the virus is an RNA virus.

In some embodiments, the virus comprises a virus from the Coronaviridae family, the Arenaviridae family, the Nairoviridae family, the Flaviviridae family, the Hepeviridae family, the Filoviridae family, or the Togaviridae family.

In some embodiments, the virus from the Coronaviridae family comprises severe acute respiratory syndrome coronavirus 2 (SARS-CoV-2, COVID-19 virus), severe acute respiratory syndrome coronavirus (SARS-CoV), human coronavirus 229E (HCoV-229E), Middle East respiratory syndrome coronavirus (MERS-CoV), human coronavirus OC43 (HCoV-OC43), human coronavirus NL63 (HCoV-NL63), or human coronavirus HKU1 (HCoV-HKU1).

In some embodiments, the virus from the Arenaviridae family comprises Lassa mammarenavirus (LASV), Guanarito mammarenavirus, Junin mammarenavirus, Lujo mammarenavirus, Machupo mammarenavirus, Sabia mammarenavirus, or Whitewater Arroyo mammarenavirus.

In some embodiments, the virus from the Nairoviridae family comprises Crimean-Congo hemorrhagic fever virus (CCHFV).

In some embodiments, the virus from the Flaviviridae family comprises Zika virus (ZIKV), hepacivirus C (hepatitis C virus, HepC), dengue fever virus, yellow fever virus, Japanese encephalitis virus, or West Nile virus.

In some embodiments, the virus from the Hepeviridae family comprises hepatitis E virus (HEV) or hepatitis B virus.

In some embodiments, the virus from the Filoviridae family comprises Ebolavirus, Marburgvirus, Dianlovirus, Cuevavirus, Striavirus, or Thamnovirus.

In some embodiments, the virus from the Togaviridae family comprises an Alphavirus. In some embodiments, the virus from the Alphavirus comprises Chikungunya virus, Eastern equine encephalitis virus, Western equine encephalitis virus, Barmah Forest virus, Mayaro virus, O'nyong'nyong virus, Ross river virus, Semliki Forest virus, Sindbis virus, Una virus, Tonate virus, or Venezuelan equine encephalitis.

In some embodiments, the rocaglate compound may be administered prophylactically before infection, may be administered after suspected or known virus exposure but prior to the appearance of symptoms of infection, administered during an incubation period of a virus, or any combination thereof.

In some embodiments, the composition further comprises a pharmaceutically acceptable carrier, excipient, or diluent.

Also disclosed herein are methods for reducing or inhibiting translation initiation of a messenger ribonucleic acid (mRNA) of a virus in a host cell or organism infected by the virus, the methods comprising administering to the cell or organism a therapeutically effective amount of a pharmaceutical composition comprising a rocaglate compound of formula (I) or a pharmaceutically acceptable salt thereof.

In some embodiment, the virus is an RNA virus.

Also disclosed herein are uses of a synthetic rocaglate composition for reducing or inhibiting translation initiation of a messenger ribonucleic acid (mRNA) of a virus in a host cell or organism infected by the virus, the synthetic rocaglate composition comprising a therapeutically effective amount of a rocaglate compound of formula (I) or a pharmaceutically acceptable salt thereof. In some embodiments, the virus is an RNA virus.

Gene expression in prokaryotic and eukaryotic cells includes the steps of transcription of deoxyribonucleic acid (DNA) into ribonucleic acid (RNA). Transcription and subsequent processing of messenger RNA (mRNA) results in a template for protein synthesis via translation of the mRNA into protein, which is then further processed. Protein synthesis includes initiation, elongation, and termination steps. Part of the initiation phase includes the binding and subsequent activity of initiation factors.

A eukaryotic example of an initiation factor, the DEAD-box RNA helicase eukaryotic initiation factor 4A (eIF4A), which is part of the heterotrimeric translation initiation complex eukaryotic initiation factor 4F (eIF4F), unwinds ribonucleic acid (RNA) secondary structures in 5′-untranslated repeats (5′-UTRs) of selected messenger ribonucleic acids (mRNAs) to enable binding of the 43S preinitiation complex (PIC). In cells, eIF4A plays a role in the translation of protooncogenic messenger ribonucleic acids (mRNAs) with complex-structured 5′-UTRs. Viral mRNAs also contain structured 5′-UTRs.

Hallmark features of eIF4A-dependent translation define specific 5′UTR elements that confer a requirement for the eIF4A RNA helicase. The key features are longer 5′UTRs, a 12-mer (GGC)4 motif, and related 9-mer variant motifs. Importantly, the 12-mer and 9-mer motifs precisely localize to between 53% and 65% of all predicted RNA G-quadruplex structures (depending on the analysis tool). The 9-mer sequences require neighboring nucleotides to complete the structure as the minimal number is 12 nucleotides, and it was frequently observed that more than 12 nucleotides contribute to the G-quadruplex. Moreover, most of the remaining G-quadruplexes are based on highly similar sequence elements. In contrast, IRES mRNAs are somewhat protected, while TOP, TOP-like, or PRTE elements do not appear to influence the eIF4A requirement. This is distinct from mTORC1 inhibition, which affects a different set of transcripts marked by TOP and TOP-like elements. These findings identify sequence motifs that represent translational control elements encoded in the 5′UTR of several hundred transcripts and that confer a requirement for eIF4A RNA helicase action.

RNA G-quadruplex structures are typically made from at least two stacks of four guanosines exhibiting non-Watson-Crick interactions (e.g., hydrogen bonds) and connected by one or more linker nucleotides. The linker is most often a cytosine and less frequently an adenosine. There is variation in the exact structural composition and sequence requirement as our examples illustrate. The minimum requirement for the structure is a (GGC/A)4 sequence and neighboring nucleotides can complete the structure.

The cap-binding protein eIF4E is limiting for cap-dependent translation and its signaling is controlled by, e.g., mTORC1 and 4E-BP. For a set of mRNAs, the eIF4A helicase activity is required and represents the point of attack for three natural compounds, Silvestrol, hippuristanol, and pateamine. Regulatory interactions occur between eIF4A and the eIF4B, eIF4G, and eIF4H factors, and between S6 kinase in the phosphorylation and signaling control of eIF4B. These interactions define a broadly relevant layer of translational control that is distinct from the control of eIF4E by 4E-BP and mTORC1.

A mechanism of translational control has been identified that is characterized by a requirement for eIF4A/DDX2 RNA helicase activity and underlies the antiviral effects of Silvestrol. eIF4A refers to eIF4A1 or eIF4A2, and RNA helicases include, but are not limited to, eIF4A1, eIF4A2, DHX9 or DHX36.

eIF4A-dependent translation-controlling motifs are typically present in the 5′ UTR of the mRNA. In certain embodiments, the eIF4A-dependent translation-controlling motif comprises a G-quadruplex structure.

In one embodiment, a rocaglate compound of formula (I) interferes with eIF4A activity. In one embodiment, a rocaglate compound of formula (I) may inhibit eIF4A helicase activity.

In one embodiment, Compound 9, Compound 8/9 racemate (a racemic mixture of Compound 8 and Compound 9), Compound 8, Compound 6, Compound 7, Compound 6/7 (a racemic mixture of Compound 6 and Compound 7), Compound 1, or Compound 5 interferes with eIF4A activity. In one embodiment, Compound 9, Compound 8/9 racemate, Compound 8, Compound 1, Compound 6, or Compound 6/7 (a racemic mixture of Compound 6 and Compound 7) interferes with eIF4A activity. In one embodiment, Compound 9, Compound 8/9 racemate, or Compound 8 interferes with eIF4A activity. In one embodiment, Compound 9, Compound 8/9 racemate, Compound 8, Compound 6, Compound 7, Compound 6/7 a racemic mixture of Compound 6 and Compound 7, Compound 1, or Compound 5 may inhibit eIF4A helicase activity. In one embodiment, Compound 9, Compound 8/9 racemate, Compound 8, Compound 1, Compound 6, or Compound 6/7 (a racemic mixture of Compound 6 and Compound 7) may inhibit eIF4A helicase activity. In one embodiment, Compound 9, Compound 8/9 racemate, or Compound 8 may inhibit eIF4A helicase activity.

Compound 1, Compound 2, Compound 3, Compound 4, Compound 5, Compound 6, Compound 7, Compound 8, Compound 9, and enantiomers of any of these, and/or racemic mixtures thereof and/or synonymic variants of any of these are contemplated herein.

“Rocaglates” are a class of compounds that act as potent inhibitors of translation initiation. In some embodiments, they are proposed to form stacking interactions with polypurine sequences in the 5′-untranslated region (UTR) of selected mRNAs, thereby clamping the RNA substrate onto eIF4A and causing inhibition of the translation initiation complex. Rocaglates include, but are not limited to, Compound 1 ((1R,2R,3S,3aR,8bS)-1,8b-dihydroxy-3a-(4-hydroxyphenyl)-N,6,8-trimethoxy-3-phenyl-2,3-dihydro-1Hcyclopenta[b]benzofuran-2-carboxamide), Compound 2 ((1S,2S,3R,3aS,8bR)-1,8b-dihydroxy-3a-(4-hydroxyphenyl)-N,6,8-trimethoxy-3-phenyl-2,3-dihydro-1Hcyclopenta[b]benzofuran-2-carboxamide), Compound 3 ((1R,2S,3R,3aR,8bS)-1,8b-dihydroxy-3a-(4-hydroxyphenyl)-N,6,8-trimethoxy-3-phenyl-2,3-dihydro-1Hcyclopenta[b]benzofuran-2-carboxamide), Compound 4 ((1S,2R,3S,3aS,8bR)-1,8b-dihydroxy-3a-(4-hydroxyphenyl)-N,6,8-trimethoxy-3-phenyl-2,3-dihydro-1Hcyclopenta[b]benzofuran-2-carboxamide), Compound 5 (sodium 4-((1R,2R,3S,3aR,8bS)-1,8b-dihydroxy-6,8-dimethoxy-2-(methoxycarbamoyl)-3-phenyl-2,3,3a,8b-tetrahydro-1H-cyclopenta[b]benzofuran-3a-yl)phenyl phosphate), Compound 6 ((1R,2R,3S,3aR,8bS)-1,6,8b-trihydroxy-N,8-dimethoxy-3a-(4-methoxyphenyl)-3-phenyl-2,3-dihydro-1Hcyclopenta[b]benzofuran-2-carboxamide), Compound 7 ((1S,2S,3R,3aS,8bR)-1,6,8b-trihydroxy-N,8-dimethoxy-3a-(4-methoxyphenyl)-3-phenyl-2,3,3a,8b-tetrahydro-1H-cyclopenta[b]benzofuran-2-carboxamide), Compound 6/7 racemate (a racemic mixture of Compound 6 [(1R,2R,3S,3aR,8bS)-1,6,8b-trihydroxy-N,8-dimethoxy-3a-(4-methoxyphenyl)-3-phenyl-2,3-dihydro-1Hcyclopenta[b]benzofuran-2-carboxamide] and Compound 7 [(1S,2S,3R,3aS,8bR)-1,6,8b-trihydroxy-N,8-dimethoxy-3a-(4-methoxyphenyl)-3-phenyl-2,3,3a,8b-tetrahydro-1H-cyclopenta[b]benzofuran-2-carboxamide]), Compound 8 ((1S,2S,3R,3aS,8bR)-1,8b-dihydroxy-3a-(4-hydroxyphenyl)-6,8-dimethoxy-3-phenyl-2,3-dihydro-1H-cyclopenta[b]benzofuran-2-carboxamide), Compound 9 ((1R,2R,3S,3aR,8bS)-1,8b-dihydroxy-3a-(4-hydroxyphenyl)-6,8-dimethoxy-3-phenyl-2,3-dihydro-1H-cyclopenta[b]benzofuran-2-carboxamide), Compound 8/9 racemate (a racemic mixture of Compound 8 [(1S,2S,3R,3aS,8bR)-1,8b-dihydroxy-3a-(4-hydroxyphenyl)-6,8-dimethoxy-3-phenyl-2,3-dihydro-1H-cyclopenta[b]benzofuran-2-carboxamide] and Compound 9 [(1R,2R,3S,3aR,8bS)-1,8b-dihydroxy-3a-(4-hydroxyphenyl)-6,8-dimethoxy-3-phenyl-2,3-dihydro-1H-cyclopenta[b]benzofuran-2-carboxamide]), among other rocaglamide ((1R,2R,3S,3aR,8bS)-1,8b-dihydroxy-6,8-dimethoxy-3a-(4-methoxyphenyl)-N,N-dimethyl-3-phenyl-2,3-dihydro-1H-cyclopenta[b][1]benzofuran-2-carboxamide) derivatives.

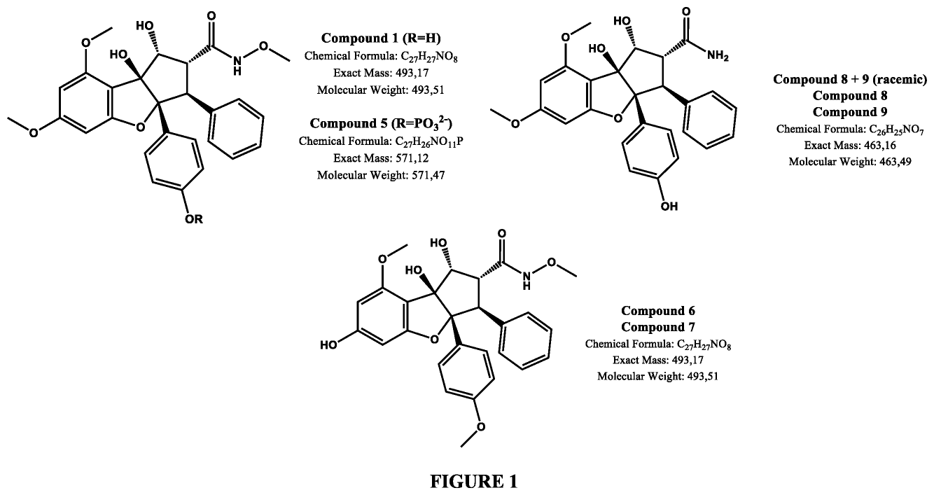

Synthetic rocaglates include, but are not limited to, Compound 1, Compound 2, Compound 3, Compound 4, Compound 5, Compound 6, Compound 7, Compound 8, Compound 9, and enantiomers of any of these, and/or racemic mixtures thereof and/or synonymic variants of any of these (see FIG. 1), such as those shown in TABLE 1.

TABLE 1
Synthetic rocaglates.

| Compound | | |
|---|---|---|
| No. | Structure | Compound name |
| 1 | 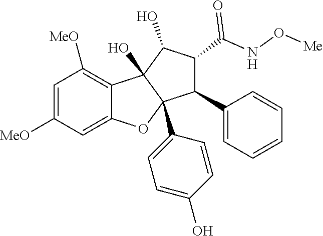 [SMILES: CONC(=O)[C@H]1[C@@H](O)[C@@]2(O)c3c(OC)cc(OC)cc3O[C@@]2(c2ccc(O)cc2)[C@@H]1c1ccccc1] | (1R,2R,3S,3aR,8bS)-1,8b-dihydroxy-3a- (4-hydroxyphenyl)-N,6,8-trimethoxy-3- phenyl-2,3-dihydro- 1Hcyclopenta[b]benzofuran-2- carboxamide |
| 2 | 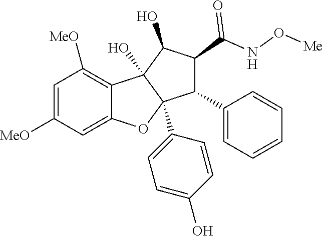 [SMILES: CONC(=O)[C@@H]1[C@H](O)[C@]2(O)c3c(OC)cc(OC)cc3O[C@]2(c2ccc(O)cc2)[C@H]1c1ccccc1] | (1S,2S,3R,3aS,8bR)-1,8b-dihydroxy-3a- (4-hydroxyphenyl)-N,6,8-trimethoxy-3- phenyl-2,3-dihydro- 1Hcyclopenta[b]benzofuran-2- carboxamide |
| 3 | 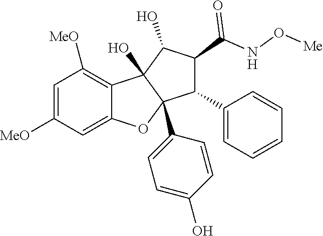 [SMILES: CONC(=O)[C@@H]1[C@@H](O)[C@@]2(O)c3c(OC)cc(OC)cc3O[C@@]2(c2ccc(O)cc2)[C@H]1c1ccccc1] | (1R,2S,3R,3aR,8bS)-1,8b-dihydroxy-3a- (4-hydroxyphenyl)-N,6,8-trimethoxy-3- phenyl-2,3-dihydro- 1Hcyclopenta[b]benzofuran-2- carboxamide |
| 4 | 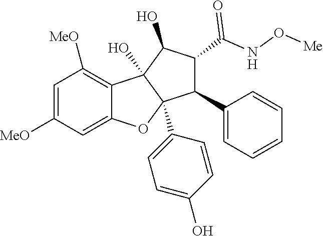 [SMILES: CONC(=O)[C@@H]1[C@@H](c2ccccc2)[C@@]2(c3ccc(O)cc3)Oc3cc(OC)cc(OC)c3[C@@]2(O)[C@H]1O] | (1S,2R,3S,3aS,8bR)-1,8b-dihydroxy-3a- (4-hydroxyphenyl)-N,6,8-trimethoxy-3- phenyl-2,3-dihydro- 1Hcyclopenta[b]benzofuran-2- carboxamide |
| 5 | 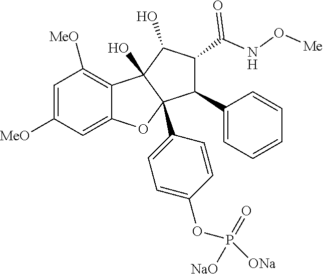 | sodium 4-((1R,2R,3S,3aR,8bS)-1,8b- dihydroxy-6,8-dimethoxy-2- (methoxycarbamoyl)-3-phenyl-2,3,3a,8b- tetrahydro-1H-cyclopenta[b]benzofuran- 3a-yl)phenyl phosphate |
| 6 | 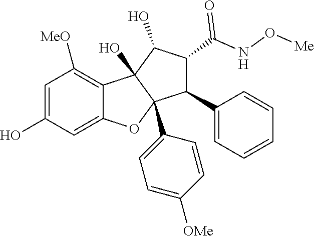 [SMILES: CONC(=O)[C@H]1[C@@H](O)[C@@]2(O)c3c(OC)cc(O)cc3O[C@@]2(c2ccc(OC)cc2)[C@@H]1c1ccccc1] | (1R,2R,3S,3aR,8bS)-1,6,8b-trihydroxy- N,8-dimethoxy-3a-(4-methoxyphenyl)-3- phenyl-2,3-dihydro- 1Hcyclopenta[b]benzofuran-2- carboxamide |
| 7 | 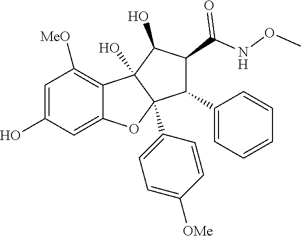 [SMILES: CONC(=O)[C@@H]1[C@H](O)[C@]2(O)c3c(OC)cc(O)cc3O[C@]2(c2ccc(OC)cc2)[C@H]1c1ccccc1] | (1S,2S,3R,3aS,8bR)-1,6,8b-trihydroxy- N,8-dimethoxy-3a-(4-methoxyphenyl)-3- phenyl-2,3,3a,8b-tetrahydro-1H- cyclopenta[b]benzofuran-2-carboxamide |
| 8 | 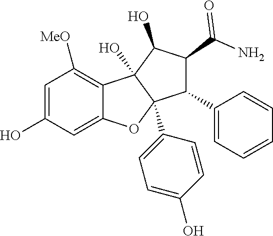 [SMILES: COc1cc(O)cc2c1[C@@]1(O)[C@@H](O)[C@@H](C(N)=O)[C@H](c3ccccc3)[C@@]1(c1ccc(O)cc1)O2] | (1S,2S,3R,3aS,8bR)-1,8b-dihydroxy-3a- (4-hydroxyphenyl)-6,8-dimethoxy-3- phenyl-2,3-dihydro-1H- cyclopenta[b]benzofuran-2-carboxamide |
| 9 | 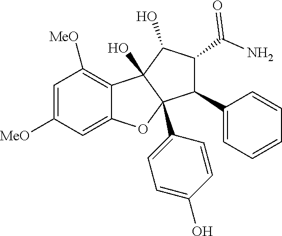 [SMILES: COc1cc(OC)c2c(c1)O[C@@]1(c3ccc(O)cc3)[C@H](c3ccccc3)[C@@H](C(N)=O)[C@@H](O)[C@@]21O] | (1R,2R,3S,3aR,8bS)-1,8b-dihydroxy-3a- (4-hydroxyphenyl)-6,8-dimethoxy-3- phenyl-2,3-dihydro-1H- cyclopenta[b]benzofuran-2-carboxamide |

Rocaglates (e.g., Compound 9, Compound 8/9, Compound 8, Compound 1, Compound 6, Compound 6/7) inhibit translation by reducing or inhibiting eIF4A activity. Reducing or inhibiting eIF4A activity can be achieved by reducing or inhibiting an eIF4A helicase activity and/or by reducing or inhibiting eIF4A clamping to a 5′-untranslated region (5′-UTR) of the mRNA of the virus. In some embodiments, the 5′-UTR comprises a hairpin structure. In some embodiments, the 5′-UTR comprises a polypurine sequence element comprising at least 10 purine nucleotides. In some embodiments, the 5′-UTR comprises a polypurine sequence element comprising at least 20 purine nucleotides. In some embodiments, the polypurine sequence element comprises at least 30 purine nucleotides.

A “virus” is a small infectious agent. While not inside an infected cell or in the process of infecting a cell, viruses exist in the form of independent particles, or virions, consisting of: (i) the genetic material (i.e., long molecules of DNA or RNA that encode the structure of the proteins by which the virus acts); (ii) a protein coat, the capsid, which surrounds and protects the genetic material; and in some cases (iii) an outside envelope of lipids. A virus has either a DNA or an RNA genome and is called a “DNA virus” or an “RNA virus,” respectively. The majority of viruses have RNA genomes. Different viruses can infect prokaryotes or eukaryotes. An “RNA virus” usually has single-stranded RNA (ssRNA) as its genetic material, but may instead have double-stranded RNA (dsRNA) as its genetic material. RNA viruses often have high mutation rates compared to DNA viruses, because viral RNA polymerases generally lack the proofreading ability of DNA polymerases. This high mutation rate often makes it difficult to construct effective vaccines against the diseases caused by RNA viruses.

Viruses cannot replicate on their own, but instead reproduce by infecting host cells and usurping the host cellular machinery, including the host transcription and/or translation machinery, to produce more virus particles. This property of viruses, as well as the ability of many viruses to mutate, makes treatment of viral infections difficult. Viral RNAs often contain highly structured 5′-UTRs, which may be eIFA-dependent.

Viruses include, but are not limited to, coronaviruses, arenaviruses, bunyaviruses, flaviviruses, and orthohepeviruses. Viruses include, but are not limited to, viruses from the Coronaviridae family, the Arenaviridae family, the Nairoviridae family, the Flaviviridae family, the Hepeviridae family, the Filoviridae family, or the Togaviridae family. Viruses include, but are not limited to, RNA viruses for which viral protein synthesis is eIF4A-dependent.

In some embodiments, the virus is from the Bunyavirales order, including, but not limited to the Arenaviridae family and/or the Nairoviridae family.

In some embodiments, the virus from the Coronaviridae family comprises human coronavirus 229E (HCoV-229E), Middle East respiratory syndrome coronavirus (MERS-CoV), severe acute respiratory syndrome coronavirus (SARS-CoV), severe acute respiratory syndrome coronavirus 2 (SARS-CoV-2, COVID-19 virus), human coronavirus OC43 (HCoV-OC43), human coronavirus NL63 (HCoV-NL63), or human coronavirus HKU1 (HCoV-HKU1).

In some embodiments, the virus from the Arenaviridae family comprises a mammarenavirus, including, but not limited to, Lassa mammarenavirus (LASV), Guanarito mammarenavirus, Junin mammarenavirus, Lujo mammarenavirus, Machupo mammarenavirus, Sabia mammarenavirus, and/or Whitewater Arroyo mammarenavirus.

In some embodiments, the virus from the Nairoviridae family comprises Crimean-Congo hemorrhagic fever virus (CCHFV).

In some embodiments, the virus is from the Flaviviridae family, including, but not limited to, the Flavivirus genus (e.g., Zika virus (ZIKV), dengue fever virus, yellow fever virus, Japanese encephalitis virus, or West Nile virus) and/or the Hepacivirus genus (e.g., hepacivirus C). In some embodiments, the virus from the Flaviviridae family comprises Zika virus (ZIKV), hepacivirus C (hepatitis C virus, HepC), dengue fever virus, yellow fever virus, Japanese encephalitis virus, or West Nile virus.

In some embodiments, the virus is from the Hepeviridae family, including, but not limited to, the Orthohepevirus genus. In some embodiments, the virus from the Hepeviridae family comprises hepatitis E virus (HEV) or hepatitis B virus.

In some embodiments, the virus is from the Filoviridae family, including, but not limited to the Ebolavirus genus (Ebola virus disease; e.g., Zaire ebolavirus, Bombali ebolavirus, Bundabugyo ebolavirus, Reston ebolavirus, Sudan ebolavirus, and Tai Forest ebolavirus), the Marburgvirus genus (Marburg virus disease; e.g., Marburg marburgvirus [Marburg virus (MARV), Ravn virus (RAVV)]), the Dianlovirus genus (Mengla virus disease; e.g., Mengla virus), the Cuevavirus genus (Lloviu virus disease; e.g., Lloviu cuevavirus), the Striavirus genus, and/or the Thamnovirus genus.

In some embodiments, the virus is from the Togaviridae family, including, but not limited to the Alphavirus genus (e.g., Chikungunya virus [Chikungunya virus disease], Eastern equine encephalitis virus [Eastern equine encephalitis], Western equine encephalitis virus [Western equine encephalitis], Barmah Forest virus, Mayaro virus, O'nyong'nyong virus, Ross River virus, Semliki Forest virus, Sindbis virus, Una virus, Tonate virus, Venezuelan equine encephalitis virus [Venezuelan equine encephalitis], and others).

Unless defined otherwise, all technical and scientific terms used herein have the same meaning as commonly understood to one of ordinary skill in the art.

As used herein, in some embodiments, the term “alkyl” refers to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkyloxy” or “alkoxy” refers to an —O-alkyl group.

In some embodiments, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. A cycloalkyl group having one or more fused aromatic rings can be attached through the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like.

As used herein, “cycloalkyloxy” refers to an —O-cycloalkyl group.

As used herein, “cycloalkylalkyl” refers to an alkyl group substituted by a cycloalkyl group.

In some embodiments, an “cycloalkylalkyloxy” group refers to an —O-alkyl group substituted by a cycloalkyl group.

In some embodiments, “aryl” refers to monocyclic or polycyclic (e.g., having 2, 3 or 4 fused rings) aromatic hydrocarbons such as, for example, phenyl, naphthyl, anthracenyl, phenanthrenyl, and the like. In some embodiments, an aryl group has from 6 to about 20 carbon atoms. In some embodiments, “aryl” may be optionally substituted at any one or more positions.

As used herein, “aryloxy” refers to an —O-aryl group.

As used herein, “arylalkyl” refers to an alkyl group substituted by an aryl group.

As used herein, “arylalkyloxy” refers to an —O-alkyl group substituted by an aryl group.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR (as in N-substituted pyrrolidinyl)).

In some embodiments, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms. In some embodiments, “heteroaryl” may be optionally substituted at any one or more positions capable of bearing a hydrogen atom.

The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, “heteroaryloxy” refers to an —O-heteroaryl group.

As used herein, “heteroarylalkyl” refers to an alkyl group substituted by a heteroaryl group.

As used herein, “heteroarylalkyloxy” refers to an —O-alkyl group substituted by a heteroaryl group.

In some embodiments, “heterocycloalkyl” refers to a non-aromatic heterocycle where one or more of the ring-forming atoms are a heteroatom such as an O, N, or S atom. Heterocycloalkyl groups can include mono- or polycyclic (e.g., having 2, 3 or 4 fused rings) ring systems as well as spirocycles. Example heterocycloalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. A heterocycloalkyl group having one or more fused aromatic rings can be attached though either the aromatic or non-aromatic portion. Also included in the definition of heterocycloalkyl are moieties where one or more ring-forming atoms are substituted by 1 or 2 oxo or sulfido groups. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “heterocycloalkyloxy” refers to an —O-heterocycloalkyl group.

As used herein, “heterocycloalkylalkyl” refers to an alkyl group substituted by a heterocycloalkyl group.

As used herein, “heterocycloalkylalkyloxy” refers to an —O-alkyl group substituted by a heterocycloalkyl group.

In some embodiments, “halo” or “halogen” includes fluoro, chloro, bromo, and iodo. A “halogen-substitution” or “halo” substitution designates replacement of one or more hydrogen atoms with F, Cl, Br or I.

In some embodiments, “haloalkyl” refers to an alkyl group having one or more halogen substituents. Example haloalkyl groups include CF₃, C₂F₅, CHF₂, CCl₃, CHCl₂, C₂Cl₅, and the like.

It is understood that each of alkyl, cycloalkyl, heterocycloalkyl, aryl, and heteroaryl may be optionally substituted with independently selected groups such as alkyl, haloalkyl, hydroxyalkyl, aminoalkyl, carboxylic acid and derivatives thereof, including esters, amides, and nitrites, hydroxy, alkyloxy, acyloxy, amino, alky and dialkylamino, acylamino, thio, and the like, and combinations thereof.

In some embodiments, the term “substituted” refers to the replacement of a hydrogen moiety with a non-hydrogen moiety in a molecule or group. It can refer to “mono-substituted” or “poly-substituted.” The term “mono-substituted” or “poly-substituted” means substituted with one or more than one substituent up to the valence of the substituted group. For example, a mono-substituted group can be substituted with 1 substituent, and a poly-substituted group can be substituted with 2, 3, 4, or 5 substituents. When a list of possible substituents is provided, the substituents can be independently selected from that group.

The term “optionally substituted,” in some embodiments, refers to that the groups in question are either unsubstituted or substituted with one or more of the substituents specified. When the groups in question are substituted with more than one substituent, the substituents may be the same or different. Such other functional groups illustratively include, but are not limited to, amino, hydroxyl, CN, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, CH, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted. In some embodiments, the functional groups are the substituents described herein for any one of variables. Furthermore, when using the terms “independently,” “independently are,” and “independently selected from” mean that the groups in question may be the same or different. Certain of the herein defined terms may occur more than once in the structure, and upon such occurrence each term shall be defined independently of the other.

Combinations of substituents envisioned by this invention are preferably those that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

In each of the foregoing and each of the following embodiments, it is to be understood that the formulas also include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds described herein may contain one or more asymmetric centers and may thus give rise to diastereomers and optical isomers. The present invention includes all such possible optical isomers, diastereomers as well as their racemic mixtures, their substantially pure resolved enantiomers, all possible geometric isomers, and pharmaceutically acceptable salts thereof. The above Formula (I) is shown without a definitive stereochemistry at certain positions. The present invention includes all stereoisomers of Formula (I) and pharmaceutically acceptable salts thereof. Further, mixtures of stereoisomers as well as isolated specific stereoisomers are also included.

The term “polynucleotide” as used herein encompasses single-stranded or double-stranded nucleic acid polymers. In certain embodiments, the nucleotides comprising the polynucleotide can be ribonucleotides or deoxyribonucleotides or a modified form of either general category of nucleotide (e.g., DNA or RNA).

The term “operably linked” encompasses components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein encompasses polynucleotide sequences that can affect expression or processing of coding sequences to which they are ligated or operably linked.

Provided herein are pharmaceutical compositions comprising a therapeutically effective amount of Compound 1, Compound 2, Compound 3, Compound 4, Compound 5, Compound 6, Compound 7, Compound 8, Compound 9, and enantiomers of any of these, and/or racemic mixtures thereof (e.g., Compound 8/9, Compound 6/7) and/or synonymic variants of any of these.

In some embodiments, the pharmaceutical composition further comprises one or more pharmaceutically acceptable carriers, excipients and/or diluents.

In some embodiments, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes “pharmaceutically acceptable salts” of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compound of the invention include the conventional non-toxic salts of the parent compound formed, for example, from non-toxic inorganic or organic acids. The pharmaceutically acceptable salts of the compound of the invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

The pharmaceutically acceptable salts of the compound of the invention can be also obtained by converting derivatives which possess tertiary amino groups into the corresponding quaternary ammonium salts in a manner known per se using quaternizing agents. Examples of suitable quaternizing agents are alkyl halides, such as methyl iodide, ethyl bromide, and n-propyl chloride, and also arylalkyl halides, such as benzyl chloride or 2-phenylethyl bromide. In some embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

Possible pharmaceutically acceptable salts are well known in the art. For example, S. M. Berge, et al. describe pharmaceutically acceptable salts in detail in J. Pharmaceutical Sciences, 66: 1-19, 1977; incorporated herein by reference.

Typically, a pharmaceutically acceptable salt form of a compound can be prepared in situ during the final isolation and purification of the compound, or separately by reacting the free base functionality with a suitable organic or inorganic acid. Examples of typical pharmaceutically acceptable, nontoxic acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods used in the art such as ion exchange.

Other pharmaceutically acceptable salts can include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and quaternary ammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

This invention further includes derivatives of the compound of the invention. The term “derivatives” includes but is not limited to ether derivatives, acid derivatives, amide derivatives, ester derivatives and the like. In addition, this invention further includes hydrates or solvates of the compound of the invention. The term “hydrate” includes but is not limited to hemihydrate, monohydrate, dihydrate, trihydrate and the like.

This invention further includes metabolites of the compound of the invention. The term “metabolite” means any substance produced from another substance by metabolism or a metabolic process.

“Pharmaceutically acceptable carriers” include any excipient which is nontoxic to the cell or subject being exposed thereto at the dosages and concentrations employed. The pharmaceutical composition may include one or additional therapeutic agents.

Pharmaceutically acceptable carriers include solvents, dispersion media, buffers, coatings, antibacterial and antifungal agents, wetting agents, preservatives, buggers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid, and thimerosal.

This invention further includes pharmaceutical compositions and pharmaceutical products of the compound of the invention. The terms “pharmaceutical composition” and “pharmaceutical product” means a composition suitable for pharmaceutical use (pharmaceutical composition), as defined herein. In some embodiments, as used herein, “pharmaceutical composition” also refers to therapeutically effective amounts of the compound of the invention together with suitable diluents, preservatives, solubilizers, emulsifiers, adjuvant and/or carriers.

In practice, the compounds of the invention, for example, represented by Formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration. e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non aqueous liquid, as an oil in water emulsion, or as a water in oil liquid emulsion.

In addition to the common dosage forms set out above, the compound represented by Formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Solid medicinal forms can comprise inert components and carrier substances, such as calcium carbonate, calcium phosphate, sodium phosphate, lactose, starch, mannitol, alginates, gelatine, guar gum, magnesium stearate, aluminum stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavorings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators. Examples of such additives are tartrate and citrate buffers, ethanol and sequestering agents (such as ethylenediaminetetraacetic acid and its nontoxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or water miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na—N-lauryl-p-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

Thus, the present invention further provides a pharmaceutical composition comprising a compound of the invention as described herein, or a pharmaceutically acceptable salt thereof, and a pharmaceutically acceptable carrier.

The term “carrier” refers to any chemical entity that can be incorporated into a composition containing an active agent (e.g., a compound of formula (I)) without interfering with the stability and/or activity of the agent. In some embodiments, the term “carrier” refers to a pharmaceutically acceptable carrier. An exemplary carrier herein is water.

Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In some embodiments, the pharmaceutical composition is administered parenterally, paracancerally, transmucosally, transdermally, intramuscularly, intravenously, intradermally, subcutaneously, intraperitoneally, intraventricularly, intravaginally, intracranially and intratumorally.

Methods of delivering drugs by pulmonary administration have been described. For example, each of U.S. Pat. Nos. 6,550,472, 6,546,927, 6,543,443, 6,540,154, 6,540,153, 6,467,476 and 6,427,682 teaches methods and devices useful in the pulmonary administration of drugs, and each is specifically incorporated herein by reference. Also, each of U.S. Pat. Nos. 6,503,480, 6,447,753, 6,387,390, 5,985,320, 5,985,309 and 5,855,913 teaches methods and devices useful in the pulmonary administration of drugs, and each is specifically incorporated herein by reference. In addition, each of U.S. Pat. Nos. 6,431,167, 6,408,854, 6,349,719, 6,167,880, 6,098,620, 5,971,951, 5,957,124, 5,906,202, 5,819,726, 5,755,218, and 5,522,385 teaches methods and devices useful in the pulmonary administration of drugs, and each is specifically incorporated herein by reference. Likewise, each of U.S. Pat. Nos. 6,546,929, 6,543,448, 6,509,006, 6,423,344, 6,303,582, and 6,138,668 teach methods of delivering drug to the lung, and each is specifically incorporated herein by reference.

This invention further includes prodrugs of the compound of the invention. Compounds of the invention can also be prepared as prodrugs, for example pharmaceutically acceptable prodrugs. The terms “pro-drug” and “prodrug” are used interchangeably herein and may refer to any compound which releases an active parent drug in vivo. Since prodrugs are known to enhance numerous desirable qualities of pharmaceuticals (e.g., solubility, bioavailability, manufacturing, etc.) the compounds of the present invention can be delivered in prodrug form. The term “prodrug” or “pro-drug” means a substance which can be converted in vivo into a biologically active agent by such reactions as hydrolysis, esterification, de-esterification, activation, salt formation and the like.

This invention further includes crystals of the compound of the invention. Further, this invention provides polymorphs of the compound of the invention. The term “crystal” means a substance in a crystalline state. The term “polymorph” refers to a particular crystalline state of a substance, having particular physical properties such as X-ray diffraction, IR spectra, melting point, and the like.

The pharmaceutical compositions of the invention may be formulated in a variety of ways, including for example, solid, semi-solid (e.g., cream, ointment, and gel), and liquid dosage forms, such as liquid solutions (e.g., topical lotion or spray), dispersions or suspensions, tablets, pills, powders, liposomes and suppositories. In some embodiments, the compositions are in the form of injectable or infusible solutions. The composition is in a form suitable for oral, intravenous, intraarterial, intramuscular, subcutaneous, parenteral, transmucosal, transdermal, or topical administration. The composition may be formulated as an immediate, controlled, extended or delayed release composition.

Pharmaceutical compositions suitable for use include sterile aqueous solutions (where water soluble) or dispersions and sterile powders for the extemporaneous preparation of sterile solutions or dispersions. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of injectable compositions can be brought about by including in the composition an agent which delays absorption, for example, aluminum monostearate and gelatin.

Sterile solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art.

Further, the preparations may be packaged and sold in the form of a kit. Such articles of manufacture will preferably have labels or package inserts indicating that the associated compositions are useful for treating a subject suffering a viral infection as described herein.

Effective doses of the compositions of the present invention, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a human, but non-human organisms, including non-human mammals and birds, as well as transgenic organisms, can also be treated. Treatment dosages may be titrated using routine methods known to those of skill in the art to optimize safety and efficacy.

In some embodiments, the compositions of the present invention may be administered prophylactically before infection, may be administered after suspected or known virus exposure but prior to the appearance of symptoms of infection, administered during an incubation period of a virus, or any combination thereof.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, species, age, sex, and weight of the individual, and the ability of the molecule to elicit a desired response in the individual. A therapeutically effective amount is also one in which any toxic or detrimental effects of the molecule are outweighed by the therapeutically beneficial effects.

Also as used herein, in some embodiments, the terms “therapeutically effective amount” and “effective amount” of an agent refer to an amount sufficient to provide a therapeutic benefit in the treatment, prevention and/or management of a disease, disorder, or condition, e.g., to delay onset of or minimize (e.g., reduce the incidence, frequency, and/or magnitude of) one or more symptoms associated with the disease, disorder or condition to be treated. Those of ordinary skill in the art will appreciate that, a composition may be said to contain a “therapeutically effective amount” of an agent if it contains an amount that is effective when administered as a single dose within the context of a therapeutic regimen. In some embodiments, a therapeutically effective amount is an amount that, when administered as part of a dosing regimen, is statistically likely to delay onset of or minimize (reduce the incidence and/or magnitude of) one or more symptoms or side effects of a disease, disorder or condition. In some embodiments, a “therapeutically effective amount” is an amount that enhances therapeutic efficacy of another agent with which the composition is administered in combination.

In some embodiments, a therapeutically effective amount for administration to a human corresponds to a reference amount (e.g., a therapeutically effective amount in an animal model such as a mouse model) adjusted for body surface area of a human as compared with body surface area of the animal model, as is known in the art (see, for example Reagan-Shaw et al., “Dose translation from animal to human studies revisited,” The FASEB Journal 22: 659-661 (2007), the entirety of which is herein incorporated by reference). In some embodiments, the reference therapeutically effective amount is an amount that is therapeutically effective in an animal model (e.g., in a mouse model). In some embodiments, the reference therapeutically effective amount is within the range of about 0.01 mg/kg to about 500 mg/kg. In some embodiments, the reference therapeutically effective amount is within the range of about 0.01 mg/kg to about 0.1 mg/kg. In some embodiments, the reference therapeutically effective amount is within the range of about 0.1 mg/kg to about 0.5 mg/kg. In some embodiments, the reference therapeutically effective amount is within the range of about 0.5 mg/kg to about 1 mg/kg. In some embodiments, the reference therapeutically effective amount is within the range of about 1 mg/kg to about 2.5 mg/kg. In some embodiments, the reference therapeutically effective amount is within the range of about 2.5 mg/kg to about 10 mg/kg. In some embodiments, the reference therapeutically effective amount is within the range of about 10 mg/kg to about 50 mg/kg. In some embodiments, the reference therapeutically effective amount is within the range of about 50 mg/kg to about 100 mg/kg. In some embodiments, the reference therapeutically effective amount is within the range of about 100 mg/kg to about 250 mg/kg. In some embodiments, the reference therapeutically effective amount is within the range of about 250 mg/kg to about 500 mg/kg.

As used herein, “modulating” refers to “stimulating” or “inhibiting” an activity of a molecular target or pathway. For example, a composition modulates the activity of a molecular target or pathway if it stimulates or inhibits the activity of the molecular target or pathway by at least 10%, by at least about 20%, by at least about 25%, by at least about 30%, by at least about 40%, by at least about 50%, by at least about 60%, by at least about 70%, by at least about 75%, by at least about 80%, by at least about 90%, by at least about 95%, by at least about 98%, or by about 99% or more relative to the activity of the molecular target or pathway under the same conditions but lacking only the presence of the composition. In another example, a composition modulates the activity of a molecular target or pathway if it stimulates or inhibits the activity of the molecular target or pathway by at least 2-fold, at least 5-fold, at least 10-fold, at least 20-fold, at least 50-fold, at least 100-fold relative to the activity of the molecular target or pathway under the same conditions but lacking only the presence of the composition. The activity of a molecular target or pathway may be measured by any reproducible means. The activity of a molecular target or pathway may be measured in vitro or in vivo. For example, the activity of a molecular target or pathway may be measured in vitro or in vivo by an appropriate assay known in the art measuring the activity. Control samples (untreated with the composition) can be assigned a relative activity value of 100%. A change in activity caused by the composition can be measured in the assays.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented.

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the invention may be administered only once, or it may be administered multiple times. For multiple dosages, the composition may be, for example, administered three times a day, twice a day, once a day, once every two days, twice a week, weekly, once every two weeks, or monthly.

As used herein, a compound “inhibits” an activity if the compound reduces the desired activity by at least 10% relative to the activity under the same conditions but lacking only the presence of the compound. The activity may be measured by any reproducible means. The activity may be measured in vitro or in vivo. In some embodiments, compounds in the methods described herein will inhibit a eIF4A activity by at least about 20%, by at least about 25%, by at least about 30%, by at least about 40%, by at least about 50%, by at least about 60%, by at least about 70%, by at least about 75%, by at least about 80%, by at least about 90%, by about 95%, by about 98%, or by about 99% or more.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Administration” to a subject is not limited to any particular delivery system and may include, without limitation, topical, transdermal, oral (for example, in capsules, suspensions or tablets), parenteral (including subcutaneous, intravenous, intramedullary, intraarticular, intramuscular, or intraperitoneal injection), or rectal. Administration to a subject may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

The term “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In some embodiments, the subject is male human or a female human.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used herein includes both one and more than one such excipient.

The present invention further provides a method for preventing, treating or intervening in the recurrence of a viral in a subject comprising administering to the subject a compound of the invention as described herein, or a pharmaceutically acceptable salt thereof, or a pharmaceutical composition thereof.

Treatment of a human or mammalian subject with a compound of the invention for any one of the aforementioned conditions or diseases is typically achieved by administration of the compound in a pharmaceutical composition. The invention also encompasses a pharmaceutical composition that is comprised of a compound of Formula (I) in combination with a pharmaceutically acceptable carrier.

The term “inhibit” is used to mean decreasing the level of activity of, including decreasing the degree of interaction with one or more biologically relevant partners (e.g., substrates, co-factors, ligands or other entities) associated with a biological effect. In some embodiments, a biologically relevant partner is one which would associate in nature.

The terms “treat” or “treating,” as used herein, refer to partially or completely alleviating, delaying onset of, reducing the incidence of, ameliorating and/or relieving a disorder, disease, or condition, or one or more symptoms of the disorder, disease or condition.

The term “patient” or “subject,” as used herein, means an animal to which a formulation or composition comprising a formulation is administered, and it includes humans and non-human mammals.

Unless otherwise indicated, all numbers expressing quantities, ratios, and numerical properties of ingredients, reaction conditions, and so forth used in the specification and claims are to be understood as being modified in all instances by the term “about”. All parts, percentages, ratios, etc. herein are by weight unless indicated otherwise.

As used herein, the singular forms “a” or “an” or “the” are used interchangeably and intended to include the plural forms as well and fall within each meaning, unless expressly stated otherwise or unless the context clearly dictates otherwise. For example, the term “a compound” or “at least one compound” may include a plurality of compounds, including mixtures thereof.

Also as used herein, “at least one” is intended to mean “one or more” of the listed elements. Singular word forms are intended to include plural word forms and are likewise used herein interchangeably where appropriate and fall within each meaning, unless expressly stated otherwise. Except where noted otherwise, capitalized and non-capitalized forms of all terms fall within each meaning.

“Consisting of” shall thus mean excluding more than traces of other elements. The skilled artisan would appreciate that while, in some embodiments the term “comprising” is used, such a term may be replaced by the term “consisting of”, wherein such a replacement would narrow the scope of inclusion of elements not specifically recited. The terms “comprises”, “comprising”, “includes”, “including”, “having” and their conjugates encompass “including but not limited to”.

The term “about” or “approximately” means within an acceptable error range for the particular value as determined by one of ordinary skill in the art, which will depend in part on how the value is measured or determined. In some embodiments, the term “about” refers to a deviance of between 0.0001-5% from the indicated number or range of numbers. In some embodiments, the term “about” refers to a deviance of between 1-10% from the indicated number or range of numbers. In some embodiments, the term “about” refers to a deviance of up to 25% from the indicated number or range of numbers. In some embodiments, the term “about” refers to ±10%.

When not otherwise stated, “substantially” means “being largely, but not wholly, that which is specified” (e.g., “substantially pure”).

The term “substantially free of,” as used herein, refers to containing no more than an insignificant amount. In some embodiments, a composition or preparation is “substantially free of” a recited element if it contains less than 5%, 4%, 3%, 2%, or 1%, by weight of the element. In some embodiments, the composition or preparation contains less than 0.9%, 0.8%, 0.7%, 0.6%, 0.5%, 0.4%, 0.3%, 0.2%, 0.1% or less of the recited element.

In some embodiments, the composition or preparation contains an undetectable amount of the recited element.

Throughout this application, various embodiments may be presented in a range format. It should be understood that the description in range format is merely for convenience and brevity and should not be construed as an inflexible limitation on the scope of certain embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Any patent, patent application publication, or scientific publication, cited herein, is incorporated by reference herein in its entirety.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Objective 1: Synthesis of (1R,2R,3S,3aR,8bS)-1,8b-dihydroxy-3a-(4-hydroxyphenyl)-N,6,8-trimethoxy-3-phenyl-2,3-dihydro-1Hcyclopenta[b]benzofuran-2-carboxamide (Compound 1), (1S,2S,3R,3aS,8bR)-1,8b-dihydroxy-3a-(4-hydroxyphenyl)-N,6,8-trimethoxy-3-phenyl-2,3-dihydro-1Hcyclopenta[b]benzofuran-2-carboxamide (Compound 2), (1R,2S,3R,3aR,8bS)-1,8b-dihydroxy-3a-(4-hydroxyphenyl)-N,6,8-trimethoxy-3-phenyl-2,3-dihydro-1Hcyclopenta[b]benzofuran-2-carboxamide (Compound 3), and 1S,2R,3S,3aS,8bR)-1,8b-dihydroxy-3a-(4-hydroxyphenyl)-N,6,8-trimethoxy-3-phenyl-2,3-dihydro-1Hcyclopenta[b]benzofuran-2-carboxamide (Compound 4)

Compounds 1-9 (see Table 1) were synthesized as described in WO 2019/161345 (published 22 Aug. 2019; PCT/US2019/018441, filed 18 Feb. 2019), which is incorporated herein by reference.

Compounds 1-4 were synthesized following the scheme below.

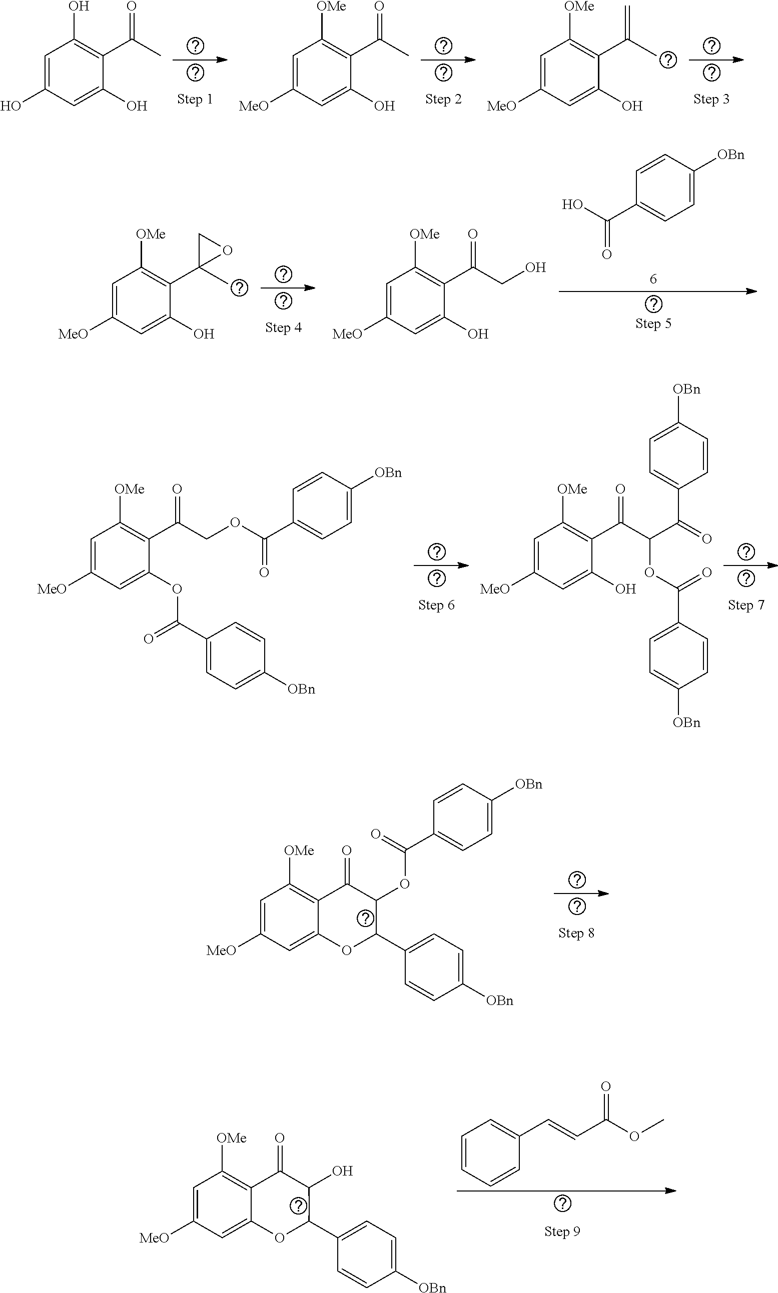

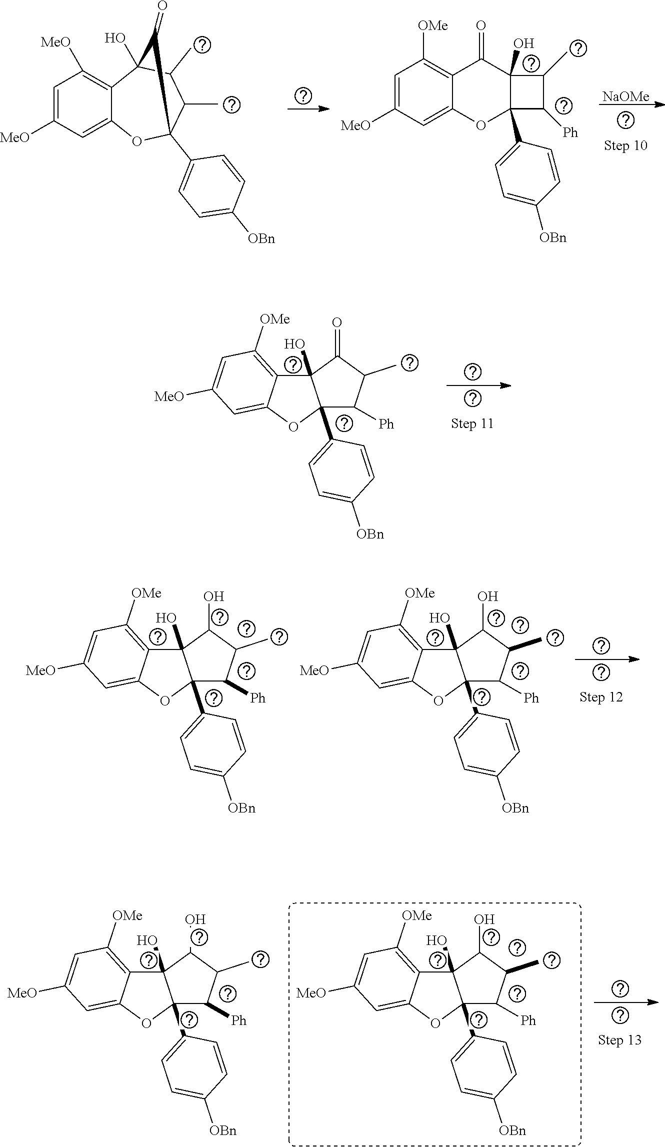

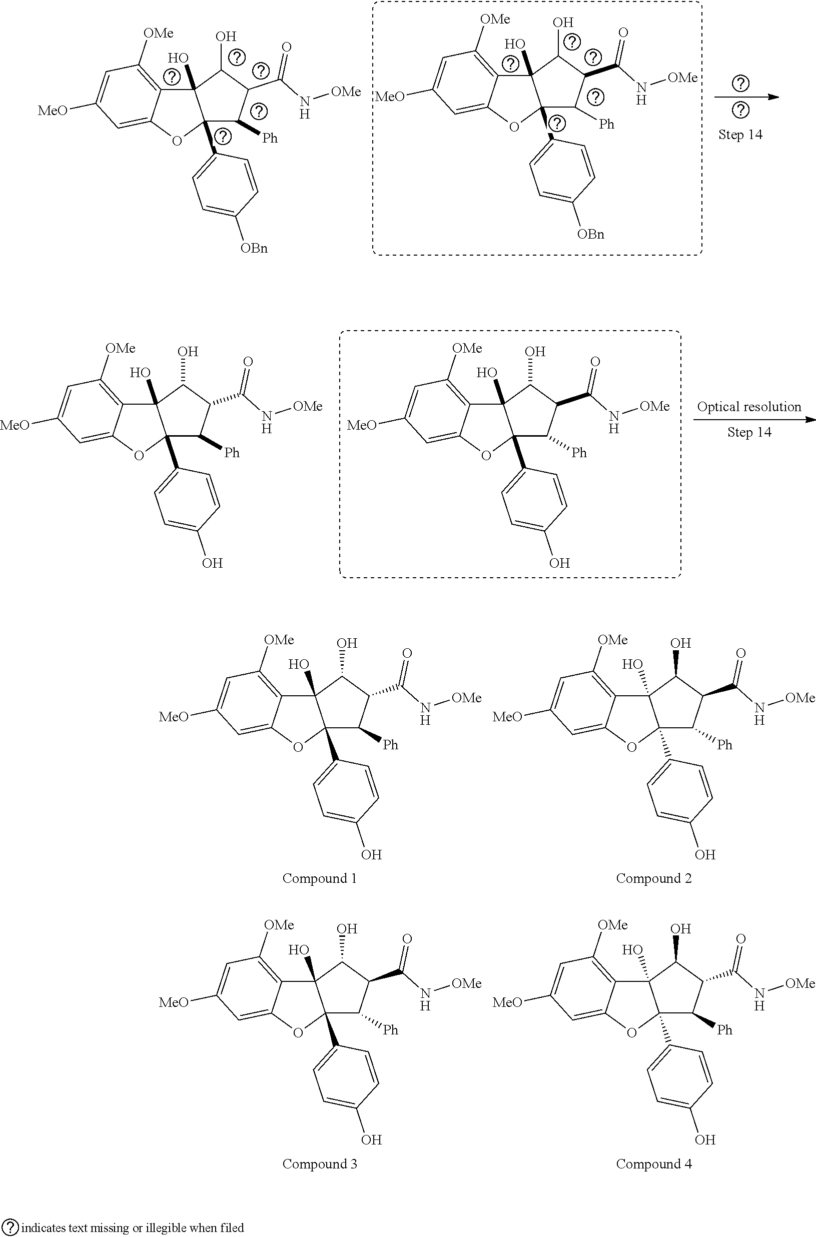

### Step 1: 1-(2-hydroxy-4,6-dimethoxy-phenyl)ethanone

To a mixture of 1-(2,4,6-trihydroxyphenyl)ethanone (50 g, 297.36 mmol, 1 eq) and K2CO3 (102.74 g, 743.40 mmol, 2.5 eq) in acetone (500 mL) was added Me2SO4 (78.76 g, 624.46 mmol, 59.22 mL, 2.1 eq) at 15° C. The mixture was stirred at 60° C. for 2 h. The reaction mixture was filtered and the filter cake was washed with EtOAc (40 mL*3). The filtrate was concentrated under reduced pressure to give 1-(2-hydroxy-4,6-dimethoxy-phenyl)ethanone (53 g, crude) as gray solid. The product will be used directly in next step without further purification.

1H NMR: (CDCl3, 400 MHz) δ=14.01 (s, 1H), 6.06 (d, J=2.2 Hz, 1H), 5.92 (d, J=2.4 Hz, 1H), 3.86 (s, 3H), 3.82 (s, 3H), 2.61 (s, 3H).

### Step 2: 2-[1-[tert-butyl(dimethyl)silyl]oxyvinyl]-3,5-dimethoxy-phenol

To a stirred solution of 1-(2-hydroxy-4,6-dimethoxy-phenyl)ethanone (53 g, 270.13 mmol, 1 eq) and TEA (82.00 g, 810.40 mmol, 112.80 mL, 3 eq) in DCM (600 mL) was added [tert-butyl(dimethyl)silyl]trifluoromethanesulfonate (142.81 g, 540.27 mmol, 124.18 mL, 2 eq) dropwise at 0° C. After the addition the reaction mixture was stirred at 0° C. for 2 h. The reaction mixture was diluted with water (500 mL) and concentrated under reduced pressure to remove the DCM. Then solution was extracted with EtOAc (200 mL*3). The organic layer was washed with brine (100 mL), dried over Na2SO4, filtered and the filtrate was concentrated under reduced pressure to give 2-[1-[tert-butyl(dimethyl)silyl]oxyvinyl]-3,5-dimethoxy-phenol (120 g, crude) as brown oil. The crude product was used directly in next step without further purification.

LCMS: (M+H+): 311.1 @1.874 min (5-95% ACN in H2O, 2.0 min).

### Step 3: 2-[2-[tert-butyl(dimethyl)silyl]oxyoxiran-2-yl]-3,5-dimethoxy-phenol

To a mixture of 2-[1-[tert-butyl(dimethyl)silyl]oxyvinyl]-3,5-dimethoxy-phenol (120 g, 316.95 mmol, 1 eq) and NaHCO3 (66.56 g, 792.37 mmol, 30.82 mL, 2.5 eq) in DCM (700 mL) was added m-CPBA (102.95 g, 507.12 mmol, 1.6 eq) at 0° C. in portions. After the addition, the mixture was stirred at 0° C. for 4 h, and then quenched by saturated aq. Na2SO3 (150 mL) and aq. NaHCO3 (100 mL). The solution was stirred at room temperature for 0.5 h. After DCM was removed by evaporation. The residual aqueous phase was extracted with EtOAc (80 mL*4). The combined organic layer was washed with brine (150 mL), dried over Na2SO4, filtered and the filtrate was concentrated under reduced pressure to give 2-[2-[tert-butyl(dimethyl)silyl]oxyoxiran-2-yl]-3,5-dimethoxy-phenol (120 g, crude) as brown oil. The crude product was used directly in next step without further purification. LCMS: (M+H+): 327.1 @2.827 min (10-80% ACN in H2O, 3.0 min).

### Step 4: 2-hydroxy-1-(2-hydroxy-4,6-dimethoxy-phenyl)ethanone

To a solution of 2-[2-[tert-butyl(dimethyl)silyl]oxyoxiran-2-yl]-3,5-dimethoxy-phenol (120 g, 367.58 mmol, 1 eq) in THF (600 mL) and H2O (60 mL) was added p-toluenesulfonic acid monohydrate (6.99 g, 36.76 mmol, 0.1 eq) at 15° C. The mixture was stirred for 80° C. at 12 h, and then quenched by saturated NaHCO3 solution (150 mL). Then the solution was concentrated under reduced pressure to remove THF. After that the mixture was filtered and the filter cake was collected. The collected solid were washed with water (20 mL*3) and EtOH (20 mL*5). Then the solid was dried under reduced pressure to give 2-hydroxy-1-(2-hydroxy-4,6-dimethoxy-phenyl)ethanone (22 g, 98.97 mmol, 26.92% yield, 95.46% purity) as gray solid.

LCMS: (M+H+): 213.0 @1.790 min (10-80% ACN in H2O, 3.0 min).

1H NMR: (CDCl3, 400 MHz) δ=13.23 (s, 1H), 6.11 (d, J=2.2 Hz, 1H), 5.94 (d, J=2.3 Hz, 1H), 4.72 (d, J=4.8 Hz, 2H), 3.88 (s, 3H), 3.85 (s, 3H), 3.77 (t, J=4.8 Hz, 1H).

### Step 5: [2-[2-(4-benzyloxybenzoyl)oxy-4,6-dimethoxy-phenyl]-2-oxo-ethyl]4-benzyloxybenzoate

To a solution of 2-hydroxy-1-(2-hydroxy-4,6-dimethoxy-phenyl)ethanone (19 g, 89.54 mmol, 1 eq) in DCM (100 mL) and DMF (150 mL) was added 4-benzyloxybenzoic acid (62.35 g, 273.17 mmol, 3.05 eq), DMAP (3.72 g, 30.44 mmol, 0.34 eq) and 3-(ethyliminomethyleneamino)-N,N-dimethyl-propan-1-amine hydrochloride (77.24 g, 402.92 mmol, 4.5 eq) at 15° C. Then the reaction mixture was stirred at 15° C. for 12 h. The reaction mixture was poured into water (400 mL) and concentrated under reduced pressure to remove DCM. Then the resulting mixture was filtered and the filter cake was washed with H2O (20 mL*3). The filter cake was collected and dried under reduced pressure. The collected solid was washed with EtOAc (20 mL*5). Then the solid was collected and dried by evaporation to give [2-[2-(4-benzyloxybenzoyl)oxy-4,6-dimethoxy-phenyl]-2-oxo-ethyl]4-benzyloxybenzoate (52 g, 73.97 mmol, 82.62% yield, 90% purity) as white solid.

1H NMR: (CDCl3, 400 MHz) δ=8.14-8.08 (m, 2H), 8.00-7.95 (m, 2H), 7.48-7.31 (m, 10H), 7.02 (d, J=8.9 Hz, 2H), 6.96 (d, J=8.9 Hz, 2H), 6.45 (d, J=2.2 Hz, 1H), 6.39 (d, J=2.1 Hz, 1H), 5.24 (s, 2H), 5.12 (d, J=11.0 Hz, 4H), 3.85 (d, J=6.7 Hz, 6H).

### Step 6: [1-(4-benzyloxybenzoyl)-2-(2-hydroxy-4,6-dimethoxy-phenyl)-2-oxo-ethyl]4-benzyloxybenzoate

To a solution of the [2-[2-(4-benzyloxybenzoyl)oxy-4,6-dimethoxy-phenyl]-2-oxo-ethyl]4-benzyloxybenzoate (20 g, 31.61 mmol, 1 eq) in THF (300 mL) at −78° C. under nitrogen was added dropwise LiHMDS (1 M, 96.00 mL, 3.04 eq). The mixture was stirred at −78° C. for 1 h and then stirred at −20° C. for 1 h. The mixture was poured into saturated aqueous NH4Cl solution (300 mL). The aqueous phase was extracted with ethyl acetate (100 mL*3). The combined organic phases were washed with brine (100 mL), dried over Na2SO4, filtered and concentrated in vacuum to give [1-(4-benzyloxybenzoyl)-2-(2-hydroxy-4,6-dimethoxy-phenyl)-2-oxo-ethyl]4-benzyloxybenzoate (20 g, crude) as gray solid. The crude product was used directly in next step without further purification.

1H NMR: (CDCl3, 400 MHz) δ=13.31 (s, 1H), 8.08 (d, J=8.8 Hz, 2H), 8.01 (d, J=8.8 Hz, 2H), 7.46-7.31 (m, 11H), 7.07 (d, J=8.8 Hz, 2H), 6.99 (d, J=8.8 Hz, 2H), 6.12 (d, J=2.2 Hz, 1H), 5.83 (d, J=2.2 Hz, 1H), 5.14 (d, J=6.6 Hz, 4H), 3.82 (s, 3H), 3.34 (s, 3H).

### Step 7: [2-(4-benzyloxyphenyl)-5,7-dimethoxy-4-oxo-chromen-3-yl]4-benzyloxy benzoate

To a stirred solution of [1-(4-benzyloxybenzoyl)-2-(2-hydroxy-4,6-dimethoxy-phenyl)-2-oxo-ethyl]4-benzyloxybenzoate (20 g, 31.61 mmol, 1 eq) in AcOH (200 mL) was added H2SO4 (15.83 g, 158.18 mmol, 8.60 mL, 98% purity, 5 eq) dropwise at 20° C. Then the mixture was stirred at 20° C. for 12 h. The reaction mixture was quenched with cooled water (200 mL) and filtered. The filter cake was washed with EtOH (40 mL*3). Then the filter cake was collected and dried under reduced pressure to give [2-(4-benzyloxyphenyl)-5,7-dimethoxy-4-oxo-chromen-3-yl]4-benzyloxy benzoate (16 g, crude) as gray solid.

1H NMR: (CDCl3, 400 MHz) δ=8.07 (d, J=8.8 Hz, 2H), 7.79 (d, J=8.9 Hz, 2H), 7.40-7.21 (m, 10H), 6.93 (t, J=8.9 Hz, 4H), 6.45 (d, J=2.2 Hz, 1H), 6.26 (d, J=2.2 Hz, 1H), 5.06 (s, 2H), 4.99 (s, 2H), 3.81 (d, J=3.3 Hz, 6H).

### Step 8: 2-(4-benzyloxyphenyl)-3-hydroxy-5,7-dimethoxy-chromen-4-one

To a stirred solution of [2-(4-benzyloxyphenyl)-5,7-dimethoxy-4-oxo-chromen-3-yl]4-benzyloxybenzoate (16 g, 26.03 mmol, 1 eq) in EtOH (160 mL) and H2O (40 mL) was added NaOH (4.16 g, 104.13 mmol, 4 eq), then the mixture was heated to 80° C. and stirred for 6 h. The reaction mixture was diluted with water (300 mL) and the solution was acidified to pH=4 with aq. HCl (2 N). The resulting mixture was filtered and the filter cake was washed with EtOH (20 mL*10). The filter cake was collected and dried under vacuum to give 2-(4-benzyloxyphenyl)-3-hydroxy-5,7-dimethoxy-chromen-4-one (9 g, crude) as light yellow solid.

LCMS: (M+H+): 405.1 @1.536 min (10-90% ACN in H2O, 2.0 min).

1H NMR: (CDCl3, 400 MHz) δ=8.17 (d, J=8.7 Hz, 2H), 7.52-7.31 (m, 6H), 7.10 (d, J=8.7 Hz, 2H), 6.52 (s, 1H), 6.33 (s, 1H), 5.14 (s, 2H), 3.96 (s, 3H), 3.90 (s, 3H).

### Step 9

To a solution of 2-(4-benzyloxyphenyl)-3-hydroxy-5,7-dimethoxy-chromen-4-one (8.5 g, 21.02 mmol, 1 eq) in DCM (850 mL), MeCN (340 mL) and MeOH (340 mL) was added methyl (E)-3-phenylprop-2-enoate (34.1 g, 210.25 mmol, 10 eq) at 3° C. The mixture was stirred and irradiated (mercury lamp radiation (400 W)) under N2 at 3° C. for 9.5 days. The reaction mixture was poured into ice water (1000 mL) and the organic phases were separated. The aqueous phase was back extracted with DCM (200 mL*3). The organic layer was washed with brine (600 mL), dried over Na2SO4, filtered and the filtrate was concentrated under reduced pressure. The residue was purified by column chromatography (SiO2, Petroleum ether/Ethyl acetate=10/1 to 5/1 to 1/1) to give the desired product (8 g, 6.85 mmol, 32.58% yield, 97% purity) as yellow solid. Due to the tautomerism, two peaks with M-17 were detected by LCMS.

LCMS: (M-17): 549.4 @1.321, 1.371 min (10-90% ACN in H2O, 2.0 min).

### Step 10

To a stirred solution of NaOMe (3.84 g, 21.32 mmol, 30% purity, 3.02 eq) in MeOH (30 mL) was added a solution of the product being obtained in the step 9 (8 g, 7.06 mmol, 1 eq) in MeOH (50 mL) at 25° C. After the addition the reaction mixture was heated to 75° C. and stirred for 2 h. The reaction was filtered after it was cooled to room temperature. The filter cake was suspended in saturated aq. NH4Cl (100 mL) and the solution was extracted with EtOAc (30 mL*5). The organic layer was washed with brine (60 mL), dried over Na2SO4, filtered and the filtrate was concentrated under reduced pressure to afford the crude product (batch 1: 2.6 g). The filtrate was concentrated under reduced pressure. The residue from the concentrated filtrate was purified by column chromatography (SiO2, Petroleum ether/Ethyl acetate=10/1 to 1/1) to give the second batch of the product (4 g). But it was still not pure after purification and used directly in next step without further purification.

LCMS: (batch 1), (M-17): 549.1@1.338; 549.0@1.398 min (10-90% ACN in H2O, 2.0 min).

LCMS: (batch 2), (M-17): 549.0@1.320 min (10-90% ACN in H2O, 2.0 min).

### Step 11: Methyl (1R,3aR,8bS)-3a-(4-benzyloxyphenyl)-1,8b-dihydroxy-6,8-dimethoxy-3-phenyl-2,3-dihydro-1Hcyclopenta[b]benzofuran-2-carboxylate

To a stirred solution of methyl (3aR,8bR)-3a-(4-benzyloxyphenyl)-8b-hydroxy-6,8-dimethoxy-1-oxo-3-phenyl-2,3-dihydrocyclopenta[b]benzofuran-2-carboxylate (4 g, 7.06 mmol, 1 eq) and AcOH (4.24 g, 70.60 mmol, 4.04 mL, 10 eq) in MeCN (30 mL) was added tetramethylammonium triacetoxyborohydride (11.14 g, 42.36 mmol, 6 eq). Then the mixture was stirred at 25° C. for 12 h. The reaction mixture was poured into ice-water (50 mL). The aqueous phase was extracted with ethyl acetate (30 mL*3). The combined organic phases were washed with brine (40 mL), dried over anhydrous Na2SO4, filtered and concentrated in vacuum to give methyl (1R,3aR,8bS)-3a-(4-benzyloxyphenyl)-1,8b-dihydroxy-6,8-dimethoxy-3-phenyl-2,3-dihydro-1Hcyclopenta[b]benzofuran-2-carboxylate (4 g, crude) was obtained as light yellow solid.

The crude product was used directly in next step without further purification.

LCMS: (M-17): 551.4@1.296 min (10-90% ACN in H2O, 2.0 min).

### Step 12: (1R,3aR,8bS)-3a-(4-benzyloxyphenyl)-1,8b-dihydroxy-6,8-dimethoxy-3-phenyl-2,3-dihydro-1H-cyclopenta[b]benzofuran-2-carboxylic acid

To a solution of the methyl (1R,3aR,8bS)-3a-(4-benzyloxyphenyl)-1,8b-dihydroxy-6,8-dimethoxy-3-phenyl-2,3-dihydro-1Hcyclopenta[b]benzofuran-2-carboxylate (4 g, 7.03 mmol, 1 eq) in THF (40 mL) and H2O (10 mL) was added LiOH·H2O (1.18 g, 28.12 mmol, 4.00 eq) at 25° C. The solution was stirred at 60° C. for 12 hours. The reaction mixture was diluted with water (50 mL). The solution was adjusted to pH=3 with aq HCl (2 N). Then the solution was extracted with EtOAc (30 mL*3). The combined organic layer was washed with brine (40 mL), dried over Na2SO4, filtered and the filtrated was concentrated under reduced pressure to afford (1R,3aR,8bS)-3a-(4-benzyloxyphenyl)-1,8b-dihydroxy-6,8-dimethoxy-3-phenyl-2,3-dihydro-1H-cyclopenta[b]benzofuran-2-carboxylic acid (3.1 g, crude) d as light yellow solid. The crude product was used directly in next step without further purification.

### Step 13: (1R,3aR,8bS)-3a-(4-benzyloxyphenyl)-1,8b-dihydroxy-N,6,8-trimethoxy-3-phenyl-2,3-dihydro-1H-cyclopenta[b]benzofuran-2-carboxamide

To a solution of (1R,3aR,8bS)-3a-(4-benzyloxyphenyl)-1,8b-dihydroxy-6,8-dimethoxy-3-phenyl-2,3-dihydro-1Hcyclopenta[b]benzofuran-2-carboxylic acid (3.1 g, 5.59 mmol, 1 eq), O-methylhydroxylamine hydrochloride (1.40 g, 16.76 mmol, 1.27 mL, 3.00 eq) in DCM (40 mL) was added HOBt (1.13 g, 8.39 mmol, 1.5 eq), EDCI (1.28 g, 6.70 mmol, 1.2 eq) and TEA (2.54 g, 25.12 mmol, 3.50 mL, 4.49 eq) at 25° C. under nitrogen. Then the mixture was stirred at 25° C. for 12 h. The reaction mixture was diluted with water (50 mL). The solution was extracted with DCM (30 mL*4). The organic layer was washed with brine (40 mL), dried over Na2SO4, filtered and the filtrate was concentrated under reduced pressure. The residue was purified by column chromatography (SiO2, Petroleum ether/Ethyl acetate=1/1 to 1/4) to give (1R,3aR,8bS)-3a-(4-benzyloxyphenyl)-1,8b-dihydroxy-N,6,8-trimethoxy-3-phenyl-2,3-dihydro-1H-cyclopenta[b]benzofuran-2-carboxamide (0.7 g, 1.18 mmol, 21.07% yield, 98.2% purity) as light yellow solid. SFC showed there were four isomers (method: IC-3_MeOH_IPAm_10-40_Gradient_4 ml. Retention time: 3.54; 3.72; 4.03; 4.28).

LCMS: (M+H+): 584.2@1.173 min (10-90% ACN in H2O, 2.0 min).

SFC: (Retention time: 3.54; 3.72; 4.03; 4.28).

### Step 14

To a solution of (1R,3aR,8bS)-3a-(4-benzyloxyphenyl)-1,8b-dihydroxy-N,6,8-trimethoxy-3-phenyl-2,3-dihydro-1Hcyclopenta[b]benzofuran-2-carboxamide (750 mg, 1.29 mmol, 1 eq) in EtOH (5 mL) and THF (5 mL) was added Pd(OH)2/C (0.5 g, 1.29 mmol, 20% purity) under N2. The suspension was degassed under vacuum and purged with H2 three times. The mixture was stirred under H2 (15 psi) at 25° C. for 8 h. The reaction mixture was filtered through a pad of Celite and the filter cake was washed with THF (20 mL×5). The filtrate was concentrated under reduced pressure to afford the crude product. The residue was checked by HPLC and Chiral SFC (Retention time: P1: 2.83 min; P2: 3.12 min; P3: 3.32 min; P4: 4.53 min).

Then the residue was separated by SFC (Instrument: Thar SFC80 preparative SFC; Column: Chiralpak AD-H 250*30 mm i.d. 5 u; Mobile phase: A for CO2 and B for IPA (0.1% NH3H2O); Gradient: B %=42%; Flow rate: 70 g/min; Wavelength: 220 nm; Column temperature: 40° C.; System back pressure: 100 bar) to give compounds 1-4.

### Compound 1

(1R,2R,3S,3aR,8bS)-1,8b-dihydroxy-3a-(4-hydroxyphenyl)-N,6,8-trimethoxy-3-phenyl-2,3-dihydro-1Hcyclopenta[b]benzofuran-2-carboxamide (125.7 mg, 238.38 μmol, 18.55% yield, 93.59% purity, 100% ee) was obtained as white solid.

LCMS: (M+H+): 494.1@ 1.562 min (25-100% ACN in H2O, 4.5 min).

1H NMR: (DMSO, 400 MHz) δ11.11 (br s, 1H), 9.28 (br s, 1H), 7.15-7.07 (m, 5H), 6.92 (dd, J=2.3, 7.1 Hz, 2H), 6.69 (d, J=8.7 Hz, 2H), 6.14 (d, J=1.8 Hz, 1H), 6.09 (d, J=2.0 Hz, 1H), 5.03 (br s, 1H), 4.84 (br s, 1H), 4.44 (d, J=10.1 Hz, 1H), 3.78 (d, J=12.7 Hz, 1H), 3.74 (s, 3H), 3.69 (s, 3H), 3.36-3.33 (m, 3H), 2.72 (dd, J=10.4, 12.5 Hz, 1H).

SFC: (Retention time: 2.78; 100% ee).

### Compound 2

(1S,2S,3R,3aS,8bR)-1,8b-dihydroxy-3a-(4-hydroxyphenyl)-N,6,8-trimethoxy-3-phenyl-2,3-dihydro-1Hcyclopenta[b]benzofuran-2-carboxamide (92.4 mg, 185.29 μmol, 14.42% yield, 98.96% purity, 97.72% % ee) was obtained as white solid.

LCMS: (M+H+): 494.1@ 2.283 min (10-80% ACN in H2O, 4.5 min).

1H NMR: (DMSO, 400 MHz) δ=11.11 (s, 1H), 9.26 (s, 1H), 7.15-7.06 (m, 5H), 6.91 (dd, J=2.3, 7.0 Hz, 2H), 6.69 (d, J=8.7 Hz, 2H), 6.14 (d, J=2.0 Hz, 1H), 6.08 (d, J=2.0 Hz, 1H), 5.02 (s, 1H), 4.83 (d, J=4.2 Hz, 1H), 4.43 (dd, J=4.2, 10.2 Hz, 1H), 3.77 (d, J=12.7 Hz, 1H), 3.74 (s, 3H), 3.69 (s, 3H), 3.33 (s, 3H), 2.74-2.66 (m, 1H).

SFC: (Retention time: 3.03; 97.72% ee).

### Compound 3

SFC showed the ee % value of P3 was 57%, so P3 was separated by SFC (Instrument: Thar SFC80 preparative SFC; Column: Chiralpak AD-H 250*30 mm id. 5 u; Mobile phase: A for CO2 and B for IPA (0.1% NH3H2O); Gradient: B %=42%; Flow rate: 70 g/min; Wavelength: 220 nm; Column temperature: 40° C.; System back pressure: 100 bar) again to give (1R,2S,3R,3aR,8bS)-1,8b-dihydroxy-3a-(4-hydroxyphenyl)-N,6,8-trimethoxy-3-phenyl-2,3-dihydro-1Hcyclopenta[b]benzofuran-2-carboxamide (43 mg, 86.17 μmol, 6.71% yield, 98.9% purity, 100% % ee) as white solid.

LCMS: (M+H+): 494.1@ 2.310 min (10-80% ACN in H2O, 4.5 min).

1H NMR: (DMSO, 400 MHz) δ11.12 (br s, 1H), 9.02 (s, 1H), 7.07-7.01 (m, 2H), 7.00-6.95 (m, 1H), 6.91-6.84 (m, 4H), 6.41 (d, J=8.4 Hz, 2H), 6.26 (s, 1H), 6.11 (s, 1H), 4.94 (s, 1H), 4.60 (d, J=3.7 Hz, 1H), 4.55 (br s, 1H), 4.13 (d, J=14.1 Hz, 1H), 3.78 (s, 3H), 3.74 (s, 3H), 3.54 (dd, J=5.0, 13.8 Hz, 1H), 3.48 (s, 3H).

SFC: (Retention time: 2.43; 100% ee).

Optical rotation: (−52.86°±7.37°; c=0.5 g/100 mL diluted with methanol, 20° C.).

### Compound 4

The product was re-purified by prep-HPLC (column: Agela Durashell C18 150*25 5 u; mobile phase: [water (10 mM NH4HCO3)-ACN]; B %: 30%-50%, 10 min) to give (1S,2R,3S,3aS,8bR)-1,8b-dihydroxy-3a-(4-hydroxyphenyl)-N,6,8-trimethoxy-3-phenyl-2,3-dihydro-1Hcyclopenta[b]benzofuran-2-carboxamide (30.5 mg, 61.80 μmol, 4.81% yield, 100% purity, 98.52% % ee) as white solid.

LCMS: (M+H+): 494.1@ 2.323 min (10-80% ACN in H2O, 4.5 min).

1H NMR: (DMSO, 400 MHz) δ=11.12 (br s, 1H), 9.04 (s, 1H), 7.08-7.01 (m, 2H), 7.00-6.94 (m, 1H), 6.91-6.84 (m, 4H), 6.41 (d, J=8.6 Hz, 2H), 6.26 (d, J=1.7 Hz, 1H), 6.11 (d, J=1.7 Hz, 1H), 4.95 (s, 1H), 4.61 (d, J=3.8 Hz, 1H), 4.57-4.52 (m, 1H), 4.13 (d, J=14.1 Hz, 1H), 3.77 (s, 3H), 3.74 (s, 3H), 3.54 (dd, J=5.7, 14.1 Hz, 1H), 3.48 (s, 3H).

SFC: (Retention time: 3.69; 98.52% ee).

Optical rotation: (54.23°±8.95°; c=0.5 g/100 mL diluted with methanol, 20° C.).

### Objective 2: Synthesis of sodium 4-((1R,2R,3S,3aR,8bS)-1,8b-dihydroxy-6,8-dimethoxy-2-(methoxycarbamoyl)-3-phenyl-2,3,3a,8b-tetrahydro-1H-cyclopenta[b]benzofuran-3a-yl)phenyl phosphate (Compound 5)

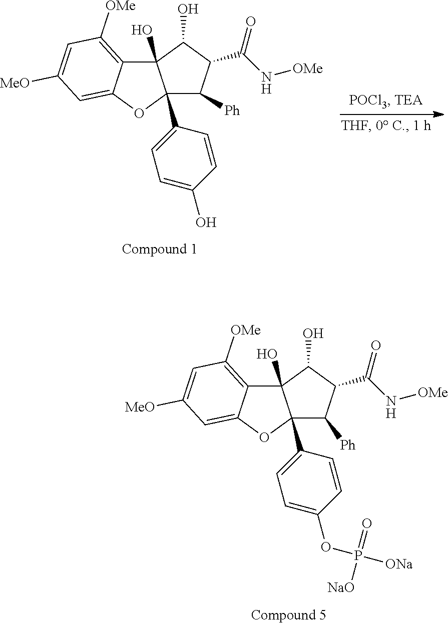

To a stirred solution of (1R,2R,3S,3aR,8bS)-1,8b-dihydroxy-3a-(4-hydroxyphenyl)-N,6,8-trimethoxy-3-phenyl-2,3-dihydro-1Hcyclopenta[b]benzofuran-2-carboxamide (43 mg, 87.13 μmol, 1 eq) in THF (5 mL) was added TEA (26 mg, 256.94 μmol, 35.76 μL, 2.95 eq) and POCl3 (67 mg, 436.96 μmol, 40.61 μL, 5.01 eq) at 0° C. Then the mixture was stirred at 0° C. for 1 h. The reaction mixture was added slowly into aq. saturated NaHCO₃ solution (2 mL). The solution was extracted with EtOAc (1 mL*2). The organic layer was discarded. The aqueous phase was purified by neutral prep-HPLC (column: Waters Xbridge 150*25 5 u; mobile phase: [Water-ACN]; B %: 1%-15%, 11 min) to give sodium 4-((1R,2R,3S,3aR,8bS)-1,8b-dihydroxy-6,8-dimethoxy-2-(methoxycarbamoyl)-3-phenyl-2,3,3a,8b-tetrahydro-1H-cyclopenta[b]benzofuran-3a-yl)phenyl phosphate (18.2 mg, 31.74 μmol, 36.42% yield, 100% purity) was obtained as light yellow solid.

LCMS: (M+H+): 574.2@ 2.041 min (10-80% ACN in H2O, 4.5 min).

1H NMR: (400 MHz, DEUTERIUM OXIDE) δ7.19-7.11 (m, 4H), 7.09-7.01 (m, 3H), 6.85 (d, J=8.6 Hz, 2H), 6.34 (d, J=1.5 Hz, 1H), 6.20 (s, 1H), 4.59 (d, J=5.1 Hz, 1H), 4.35 (d, J=14.4 Hz, 1H), 3.85-3.79 (m, 4H), 3.75 (s, 3H), 3.57 (s, 3H).

SFC: (Retention time: 1.85; % ee: 100%).

Optical rotation: (−83.13°±8.95°; c=0.5 g/100 mL diluted with water, 20° C.).

### Objective 3: Synthesis of (1R,2R,3S,3aR,8bS)-1,6,8b-trihydroxy-N,8-dimethoxy-3a-(4-methoxyphenyl)-3-phenyl-2,3-dihydro-1Hcyclopenta[b]benzofuran-2-carboxamide (Compound 6) and (1S,2S,3R,3aS,8bR)-1,6,8b-trihydroxy-N,8-dimethoxy-3a-(4-methoxyphenyl)-3-phenyl-2,3,3a,8b-tetrahydro-1H-cyclopenta[b]benzofuran-2-carboxamide (Compound 7)

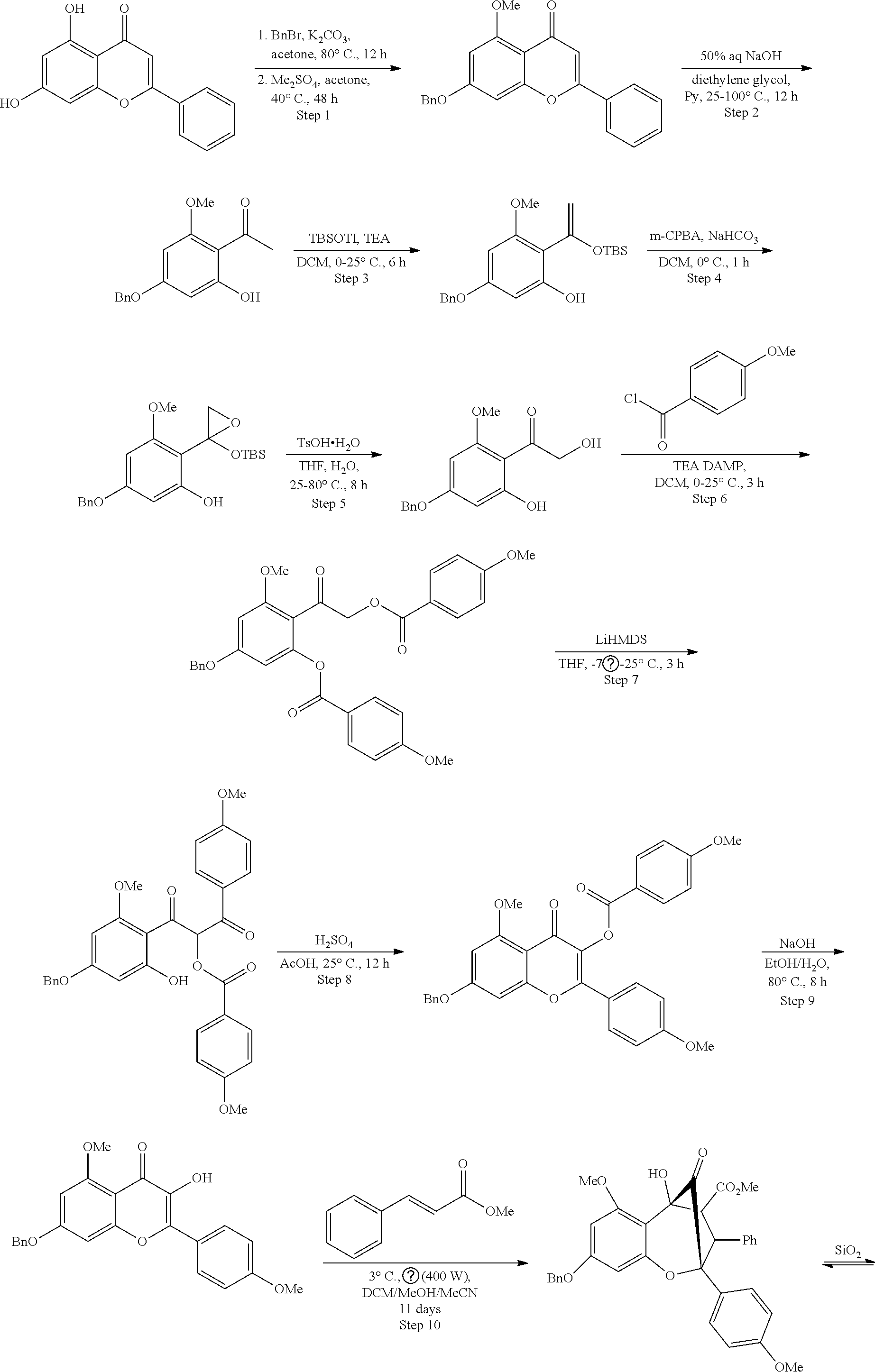

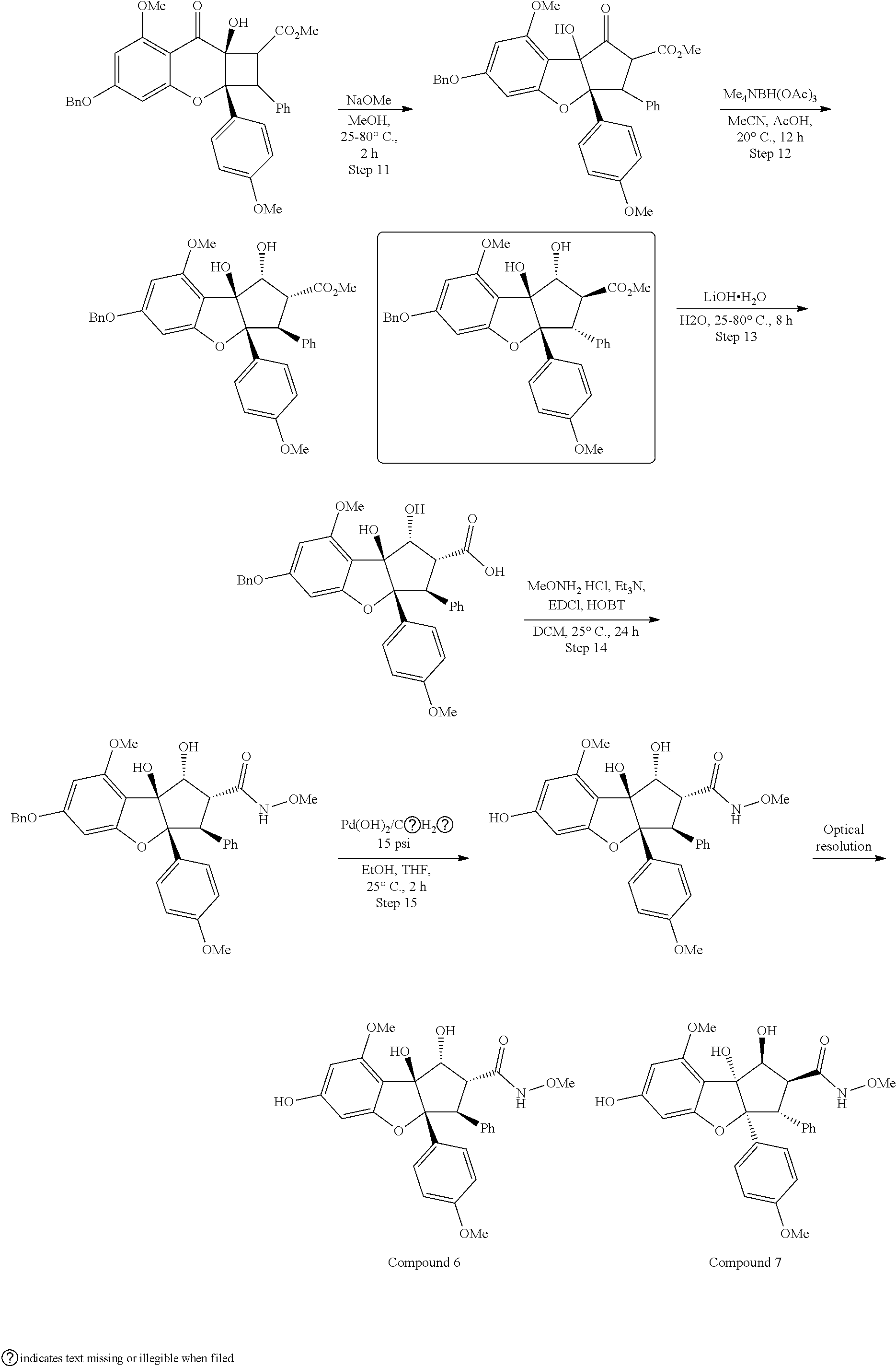

### Step 1: 7-benzyloxy-5-methoxy-2-phenyl-chromen-4-one

A suspension of 5,7-dihydroxy-2-phenyl-chromen-4-one (25 g, 98.33 mmol, 1 eq) in acetone (300 mL) was treated with K2C03 (40.77 g, 295.00 mmol, 3 eq) and bromomethylbenzene (16.82 g, 98.33 mmol, 11.68 mL, 1 eq). The mixture was heated to 60° C. and stirred for 12 h. And then it was cooled to 40° C., treated with dimethyl sulfate (37.00 g, 293.35 mmol, 27.82 mL, 2.98 eq), and stirred for 48 h at 40° C. The mixture was cooled to room temperature and the solid was removed by filtration. The cake was dissolved in water (1 L) and the solution was extracted with DCM (200 mL*3). The organic layer was combined with the filtrate that was previously concentrated to dryness under reduced pressure. The residue was washed with MTBE (100 mL*4) to give 7-benzyloxy-5-methoxy-2-phenyl-

chromen-4-one (90% purity) (63 g, 90% purity, combined with another run of the same reaction) as light green solid.

LCMS: (M+H+): 359.0 @ 1.605 min (10-90% ACN in H2O, 2.0 min).

1H NMR: (400 MHz, CDCl3) δ 7.92-7.83 (m, 2H), 7.59-7.33 (m, 8H), 6.69 (s, 1H), 6.67 (d, J=2.0 Hz, 1H), 6.47 (d, J=2.0 Hz, 1H), 5.17 (s, 2H), 3.96 (s, 3H).

### Step 2: 1-(4-benzyloxy-2-hydroxy-6-methoxy-phenyl)ethanone

7-benzyloxy-5-methoxy-2-phenyl-chromen-4-one (48 g, 133.93 mmol, 1 eq) was added to a mixture of NaOH (375 g, 4.69 mol, 50% purity, 35 eq) and pyridine (211.68 g, 2.68 mol, 216 mL, 19.98 eq) at 25° C. The mixture was vigorously stirred and treated with diethylene glycol (284.48 g, 2.68 mol, 254 mL, 20.02 eq) at 25° C. Then the reaction mixture was heated to 100° C. and stirred for 12 h. After the reaction mixture was cooled to 25° C., it was acidified to pH=1 with 8 N aqueous hydrochloric acid solution. The aqueous portion was extracted with ethyl acetate (100 mL*4). The combined organic phase was washed with brine (200 mL), dried over Na2SO4, filtered and the filtrate was concentrated under reduced pressure. The residue was washed with ice methyl alcohol (20 mL*5). The collected solid was dried under reduced pressure to give 1-(4-benzyloxy-2-hydroxy-6-methoxy-phenyl)ethanone (31.5 g, with another run of the same reaction) as light yellow solid.

1H NMR: (400 MHz, CDCl3) δ 14.05 (s, 1H), 7.46-7.33 (m, 5H), 6.15 (d, J=2.2 Hz, 1H), 6.02 (d, J=2.2 Hz, 1H), 5.07 (s, 2H), 3.85 (s, 3H), 2.62 (s, 3H).

### Step 3: 5-benzyloxy-2-[1-[tert-butyl(dimethyl)silyl]oxyvinyl]-3-methoxy-phenol

A solution of 1-(4-benzyloxy-2-hydroxy-6-methoxy-phenyl)ethanone (26.5 g, 97.32 mmol, 1 eq) and TEA (24.62 g, 243.30 mmol, 33.86 mL, 2.5 eq) in DCM (300 mL) was cooled to 0° C. Then [tert-butyl(dimethyl)silyl]trifluoromethanesulfonate (51.45 g, 194.64 mmol, 44.74 mL, 2 eq) was added drop-wise to the mixture at 0° C. under N2. After the addition, the reaction mixture was allowed to warm to 25° C. and stirred for 6 h. The reaction mixture was quenched with sat. aq NaHCO₃ (300 mL). The mixture was extracted with DCM (100 mL*2) and the separated organic layer was washed with brine (100 mL), dried over Na2SO4. After filtration, the filtrate was concentrated under reduced pressure to afford 5-benzyloxy-2-[1-[tert-butyl(dimethyl)silyl]oxyvinyl]-3-methoxy-phenol (60 g, crude) as brown oil. The crude product will be used directly in next step without further purification.

TLC Information: (PE/EtOAc=5/1)

Reactant: Rf=0.6

Product: Rf=0.8

### Step 4: 5-benzyloxy-2-[2-[tert-butyl(dimethyl)silyl]oxyoxiran-2-yl]-3-methoxy-phenol

To a mixture of 5-benzyloxy-2-[l-[tert-butyl(dimethyl)silyl]oxyvinyl]-3-methoxy-phenol (60 g, 155.22 mmol, 1 eq) and NaHCO3 (32.60 g, 388.04 mmol, 15.09 mL, 2.5 eq) in DCM (600 mL) was added m-CPBA (47.27 g, 232.83 mmol, 85% purity, 1.5 eq) in portions at 0° C. After the addition, the reaction mixture was stirred at 0° C. for 1 h, and then quenched by saturated aq. Na2SO3 (500 mL) and aq. NaHCO3 (100 mL). Then the mixture was extracted with EtOAc (200 mL*4). The organic layer was washed with brine (300 mL), dried over Na2SO4, filtered and the filtrate was concentrated under reduced pressure to give 5-benzyloxy-2-[2-[tert-butyl(dimethyl)silyl]oxyoxiran-2-yl]-3-methoxy-phenol (66 g, crude) as brown oil. The crude product was used directly in next step without further purification.

TLC Information: (PE/EtOAc=10/1)

Reactant: Rf=0.5

Product: Rf=0.3

### Step 5: 1-(4-benzyloxy-2-hydroxy-6-methoxy-phenyl)-2-hydroxy-ethanone

To a solution of 5-benzyloxy-2-[2-[tert-butyl(dimethyl)silyl]oxyoxiran-2-yl]-3-methoxy-phenol (66 g, 163.95 mmol, 1 eq) in THF (600 mL) and H2O (60 mL) was added 4-methylbenzenesulfonic acid hydrate (3.12 g, 16.41 mmol, 0.1 eq) at 25° C. The mixture was stirred for 80° C. at 6 hours. The reaction mixture was quenched with saturated aqueous sodium bicarbonate solution (500 mL) and concentrated under reduced pressure to remove THF. Then the residue aqueous phase was extracted with EtOAc (150 mL*4). The organic phase was washed with brine (300 mL), dried over Na2SO4, filtered and the filtrate was concentrated under reduced pressure. The residue solid was washed with EtOH (40 mL*4). The collected solid was dried under reduced pressure to give 1-(4-benzyloxy-2-hydroxy-6-methoxy-phenyl)-2-hydroxy-ethanone (91.29% purity) (18 g, combined with another run of the same reaction) as gray solid.

LCMS: (M+H+): 289.2 @1.170 min (5-95% ACN in H2O, 2.0 min).

1H NMR: (400 MHz, CDCl3) δ 13.22 (s, 1H), 7.46-7.32 (m, 5H), 6.20 (d, J=2.4 Hz, 1H), 6.03 (d, J=2.2 Hz, 1H), 5.09 (s, 2H), 4.72 (d, J=4.6 Hz, 2H), 3.86 (s, 3H), 3.77 (br s, 1H).

### Step 6: [2-[4-benzyloxy-2-methoxy-6-(4-methoxybenzoyl)oxy-phenyl]-2-oxo-ethyl]4-methoxybenzoate

A solution of 1-(4-benzyloxy-2-hydroxy-6-methoxy-phenyl)-2-hydroxy-ethanone (7 g, 24.28 mmol, 1 eq) in DCM (100 mL) was treated with DMAP (149 mg, 1.22 mmol, 5.02e-2 eq) and TEA (7.4 g, 73.13 mmol, 10.18 mL, 3.01 eq). The mixture was cooled to 0° C., treated with 4-methoxybenzoyl chloride (8.3 g, 48.65 mmol, 6.69 mL, 2 eq) dropwise. After the addition, the mixture was allowed to warm to 25° C. and stirred for 3 h. The reaction mixture was quenched with 1 N aqueous hydrochloric acid (100 mL), and the organic layer was separated. The aqueous phase was back-extracted with DCM (40 mL*3). The combined organic phases were washed with brine (100 mL), dried over Na2SO4, filtered and the filtrate was concentrated under reduced pressure. The residue was washed with EtOH (20 mL*3). The collected solid was dried under recued pressure to give [2-[4-benzyloxy-2-methoxy-6-(4-methoxybenzoyl)oxy-phenyl]-2-oxo-ethyl]4-methoxybenzoate (25 g, 44.92 mmol, 92.50% yield) as white solid.

1H NMR: (400 MHz, CDCl3)δ 8.14-8.09 (m, 2H), 8.00-7.96 (m, 2H), 7.45-7.36 (m, 5H), 6.97-6.92 (m, 2H), 6.91-6.87 (m, 2H), 6.56 (d, J=2.1 Hz, 1H), 6.48 (d, J=2.2 Hz, 1H), 5.25 (s, 2H), 5.08 (s, 2H), 3.86 (s, 3H), 3.84 (d, J=2.0 Hz, 6H).

### Step 7: [1-(4-benzyloxy-2-hydroxy-6-methoxy-benzoyl)-2-(4-methoxyphenyl)-2-oxo-ethyl]4-methoxybenzoate

A stirred solution of [2-[4-benzyloxy-2-methoxy-6-(4-methoxybenzoyl)oxy-phenyl]-2-oxo-ethyl]4-methoxybenzoate (25 g, 44.92 mmol, 1 eq) in THF (300 mL) was cooled to −70° C. and was treated with LiHMDS (1 M, 135 mL, 3.01 eq) dropwise. The mixture was allowed to warm to 25° C. and stirred for 3 h. The mixture was poured into saturated aqueous NH4Cl solution (400 mL). The aqueous phase was extracted with ethyl acetate (100 mL*3). The combined organic phases were washed with brine (100 mL), dried over Na2SO4, filtered and concentrated in vacuum to give [1-(4-benzyloxy-2-hydroxy-6-methoxy-benzoyl)-2-(4-methoxyphenyl)-2-oxo-ethyl]4-methoxybenzoate (25 g, crude) as gray solid. The crude product will be used directly in next step without further purification.

1H NMR: (400 MHz, CDCl3) δ 13.30 (s, 1H), 8.10-8.06 (m, 2H), 8.04-7.99 (m, 2H), 7.43-7.37 (m, 6H), 7.00-6.97 (m, 2H), 6.94-6.90 (m, 2H), 6.20 (d, J=2.2 Hz, 1H), 5.92 (d, J=2.2 Hz, 1H), 5.06 (s, 2H), 3.88 (s, 3H), 3.85 (s, 3H), 3.34 (s, 3H).

### Step 8: [7-benzyloxy-5-methoxy-2-(4-methoxyphenyl)-4-oxo-chromen-3-yl]4-methoxybenzoate

To a suspension of [1-(4-benzyloxy-2-hydroxy-6-methoxy-benzoyl)-2-(4-methoxyphenyl)-2-oxo-ethyl]4-methoxybenzoate (25 g, 44.92 mmol, 1 eq) in AcOH (300 mL) was added H2SO4 (22.08 g, 225.13 mmol, 12 mL, 5.01 eq) at 25° C. Then the mixture was stirred at 25° C. for 12 h. The reaction mixture was quenched with cooled water (400 mL) and stirred for 10 min. It was filtered. The filter cake was washed with EtOH (30 mL*5). Then the filter cake was collected and dried under reduced pressure to give [7-benzyloxy-5-methoxy-2-(4-methoxyphenyl)-4-oxo-chromen-3-yl]4-methoxybenzoate (18 g, 33.42 mmol, 74.41% yield) as light yellow solid. The crude product was used directly in next step without further purification.

LCMS: (M+H+): 539.1 @1.683 min (10-90% ACN in H2O, 2.0 min).

1H NMR: (400 MHz, CDCl3) δ 8.20-8.14 (m, 2H), 7.91-7.85 (m, 2H), 7.50-7.36 (m, 5H), 6.99-6.92 (m, 4H), 6.64 (d, J=2.2 Hz, 1H), 6.45 (d, J=2.2 Hz, 1H), 5.16 (s, 2H), 3.91 (s, 3H), 3.89 (s, 3H), 3.83 (s, 3H).

### Step 9: 7-benzyloxy-3-hydroxy-5-methoxy-2-(4-methoxyphenyl)chromen-4-one

A suspension of [7-benzyloxy-5-methoxy-2-(4-methoxyphenyl)-4-oxo-chromen-3-yl]4-methoxybenzoate (18 g, 33.42 mmol, 1 eq) in EtOH (200 mL) was treated with NaOH (53.48 g, 66.85 mmol, 5% purity, 2 eq). The suspension was heated to 80° C. and stirred for 6 h. The reaction mixture was diluted with water (100 mL) and the solution was acidified to pH=5 with aq. HCl (2 N). Then the mixture was filtered and the filter cake was washed with EtOH (20 mL*5). The filter cake was collected and dried under vacuum to give the crude product. The residue was purified by column chromatography (SiO2, DCM/Ethyl acetate=10/1 to 5/1) to give 7-benzyloxy-3-hydroxy-5-methoxy-2-(4-methoxyphenyl)chromen-4-one (11 g, 27.20 mmol, 81.38% yield) as yellow solid.

LCMS: (M+H+): 405.2 @ 1.427 min (10-90% ACN in H2O, 2.0 min).

1H NMR: (400 MHz, CDCl3) δ 8.17 (d, J=8.3 Hz, 2H), 7.52-7.34 (m, 6H), 7.03 (d, J=8.3 Hz, 2H), 6.63 (br s, 1H), 6.43 (s, 1H), 5.15 (s, 2H), 3.97 (s, 3H), 3.89 (s, 3H).

### Step 10

A solution of 7-benzyloxy-3-hydroxy-5-methoxy-2-(4-methoxyphenyl)chromen-4-one (5 g, 12.36 mmol, 1 eq) and methyl (E)-3-phenylprop-2-enoate (23.06 g, 142.18 mmol, 11.5 eq) in DCM (500 mL), MeOH (200 mL), and MeCN (200 mL) was placed in a jacketed flask. Then the stirring mixture was cooled to 3° C. and irradiated (mercury lamp radiation (400 W)) under N2 for 11 days. The reaction mixture was poured into ice water (500 mL) and the organic phases were separated. The aqueous phase was back extracted with DCM (200 mL*3). The organic layer was washed with brine (600 mL), dried over Na2SO4, filtered and the filtrate was concentrated under reduced pressure. The residue was purified by column chromatography (SiO2, Petroleum ether/Ethyl acetate=10/1 to 5/1 to 1/1) to give the desired product (5.5 g, crude) as orange solid which was used in the next step directly.

### Step 11

To a stirred solution of NaOMe (4.77 g, 26.47 mmol, 30% purity, 3 eq) in MeOH (10 mL) was added a solution of the product of Step 10 (5.00 g, 8.82 mmol, 1 eq) in MeOH (30 mL) at 25° C. Then the mixture was stirred at 80° C. for 2 h. The reaction mixture was filtered and the filter cake was washed with MeOH (5 mL*4). The collected solid was re-dissolved in saturated aq. NH4Cl solution (50 mL) and the solution was extracted with EtOAc (20 mL*4). The organic layer was washed with brine (40 mL), dried over Na2SO4, filtered and the filtrate was concentrated under reduced pressure. Desired product (2.1 g, 3.19 mmol, 36.12% yield, 86% purity) was obtained as light yellow solid. The crude product was used directly in next step without further purification. Due to the tautomerism of the structure, two peaks were detected on LCMS.

LCMS: (M-17): 549.3@1.277 min (5-95% ACN in H2O, 2.0 min).

### Step 12: Methyl 6-benzyloxy-1,8b-dihydroxy-8-methoxy-3a-(4-methoxyphenyl)-3-phenyl-2,3-dihydro-1H-cyclopenta[b]benzofuran-2-carboxylate

To a stirred solution of starting material (2.10 g, 3.71 mmol, 1 eq) and AcOH (2.23 g, 37.07 mmol, 2.12 mL, 10 eq) in MeCN (30 mL) was added tetramethylammonium; triacetoxyboranuide (5.85 g, 22.24 mmol, 6 eq). Then the mixture was stirred at 25° C. for 12 h. The reaction mixture was poured into ice-water (60 mL). The aqueous phase was extracted with ethyl acetate (30 mL*4). The combined organic phases were washed with brine (50 mL), dried over anhydrous Na2SO4, filtered and concentrated in vacuum to give methyl 6-benzyloxy-1,8b-dihydroxy-8-methoxy-3a-(4-methoxyphenyl)-3-phenyl-2,3-dihydro-1H-cyclopenta[b]benzofuran-2-carboxylate (1.7 g, crude) as light yellow solid. The crude product was used directly in next step without further purification.

LCMS: (M-17): 551.3, (M+23): 591.2@1.240 min, (5-95% ACN in H2O, 2.0 min).

### Step 13: 6-benzyloxy-1,8b-dihydroxy-8-methoxy-3a-(4-methoxyphenyl)-3-phenyl-2,3-dihydro-1H-cyclopenta[b]benzofuran-2-carboxylic acid

To a solution of the methyl 6-benzyloxy-1,8b-dihydroxy-8-methoxy-3a-(4-methoxyphenyl)-3-phenyl-2,3-dihydro-1Hcyclopenta[b]benzofuran-2-carboxylate (1.70 g, 2.99 mmol, 1 eq) in THF (20 mL) and H2O (5 mL) was added LiOH·H2O (503.00 mg, 11.99 mmol, 4.01 eq) at 25° C. The solution was stirred at 60° C. for 6 hours. The reaction mixture was diluted with water (50 mL). The solution was adjusted to pH=3 with aq HCl (2 N). Then the solution was extracted with EtOAc (20 mL*3). The combined organic layer was washed with brine (30 mL), dried over Na2SO4, filtered and the filtrated was concentrated under reduced pressure to afford 6-benzyloxy-1,8b-dihydroxy-8-methoxy-3a-(4-methoxyphenyl)-3-phenyl-2,3-dihydro-1H-cyclopenta[b]benzofuran-2-carboxylic acid (1.7 g, crude) as light yellow solid.

LCMS: (M-17): 537.3@ 1.218 min (5-95% ACN in H2O, 2.0 min).

SFC: (Retention time: 3.54; 3.72; 4.03; 4.28).

### Step 14: 6-benzyloxy-1,8b-dihydroxy-N,8-dimethoxy-3a-(4-methoxyphenyl)-3-phenyl-2,3-dihydro-1H-cyclopenta[b]benzofuran-2-carboxamide

To a solution of 6-benzyloxy-1,8b-dihydroxy-8-methoxy-3a-(4-methoxyphenyl)-3-phenyl-2,3-dihydro-1Hcyclopenta[b]benzofuran-2-carboxylic acid (1.70 g, 3.07 mmol, 1 eq), O-methyl hydroxylamine; hydrochloride (768.02 mg, 9.20 mmol, 698.20 uL, 3 eq) in DCM (30 mL) was added HOBt (620.98 mg, 4.60 mmol, 1.5 eq), EDCI (705.99 mg, 3.68 mmol, 1.2 eq) and TEA (1.40 g, 13.81 mmol, 1.92 mL, 4.5 eq) at 25° C. Then the mixture was stirred at 25° C. for 24 h. The reaction mixture was diluted with water (100 mL). The solution was extracted with DCM (40 mL*4). The organic layer was washed with brine (50 mL), dried over Na2SO4, filtered and the filtrate was concentrated under reduced pressure. The residue was purified by column chromatography (SiO2, Petroleum ether/Ethyl acetate=1/1 to 1/4) to give 6-benzyloxy-1,8b-dihydroxy-N,8-dimethoxy-3a-(4-methoxyphenyl)-3-phenyl-2,3-dihydro-1H-cyclopenta[b]benzofuran-2-carboxamide (410 mg, 702.50 μmol, 22.92% yield) as light yellow solid.

### Step 15: (1R,2R,3S,3aR,8bS)-1,6,8b-trihydroxy-N,8-dimethoxy-3a-(4-methoxyphenyl)-3-phenyl-2,3-dihydro-1H-cyclopenta[b]benzofuran-2-carboxamide and (1S,2S,3R,3aS,8bR)-1,6,8b-trihydroxy-N,8-dimethoxy-3a-(4-methoxyphenyl)-3-phenyl-2,3,3a,8b-tetrahydro-1H-cyclopenta[b]benzofuran-2-carboxamide

To a solution of 6-benzyloxy-1,8b-dihydroxy-N,8-dimethoxy-3a-(4-methoxyphenyl)-3-phenyl-2,3-dihydro-1Hcyclopenta[b]benzofuran-2-carboxamide (410.00 mg, 702.50 μmol, 1 eq) in EtOH (5 mL) and EtOAc (5 mL) was added Pd(OH)2/C (0.2 g, 20% purity) under N2. The suspension was degassed under vacuum and purged with H2 three times. The mixture was stirred under H2 (15 psi) at 25° C. for 2 hours. The reaction mixture was filtered through a pad of Celite and the filter cake was washed with EtOAc (10 mL×5). The filtrate was concentrated under reduced pressure to afford the crude product. The residue was purified by neutral prep-HPLC (column: Waters Xbridge 150*25 5 u; mobile phase: [water (10 mM NH4HCO3)-ACN]; B %: 15%-35%, 11 min). The purified product was checked by chiral SFC (Retention time: P1: 2.71 min; P2: 3.85 min), and each isomer was separated by chiral SFC to give P1 and P2.

### Compound 6: (1R,2R,3S,3aR,8bS)-1,6,8b-trihydroxy-N,8-dimethoxy-3a-(4-methoxyphenyl)-3-phenyl-2,3-dihydro-1Hcyclopenta[b]benzofuran-2-carboxamide

(75.6 mg, 153.19 μmol, 21.81% yield, 100% purity: 100% ee) as white solid.

LCMS: (M+H+): 494.0@ 2.278 min (10-80% ACN in H2O, 4.5 min)

1H NMR: (400 MHz, METHANOL-d4) δ7.33 (d, J=8.9 Hz, 2H), 7.15-7.11 (m, 3H), 6.98 (dd, J=3.5, 5.9 Hz, 2H), 6.88 (d, J=8.9 Hz, 2H), 6.05 (d, J=1.7 Hz, 1H), 5.99 (d, J=1.8 Hz, 1H), 4.65 (d, J=10.3 Hz, 1H), 3.96 (d, J=12.6 Hz, 1H), 3.77 (d, J=2.1 Hz, 6H), 3.43 (s, 3H), 2.82 (dd, J=10.3, 12.6 Hz, 1H).

SFC: (Retention time: 2.61; % ee: 100%).

Optical rotation: (−37.43°±1.65°; c=0.5 g/100 mL diluted with methanol, 20° C.).

### Compound 7: (1S,2S,3R,3aS,8bR)-1,6,8b-trihydroxy-N,8-dimethoxy-3a-(4-methoxyphenyl)-3-phenyl-2,3,3a,8b-tetrahydro-1H-cyclopenta[b]benzofuran-2-carboxamide

(74.1 mg, 149.76 μmol, 21.32% yield, 99.74% purity, % ee: 100%) obtained as white solid.

LCMS: (M+H): 494.0@2.277 min (10-80% ACN in H2O, 4.5 min).

1H NMR: (400 MHz, METHANOL-d4) δ 7.34 (d, J=8.9 Hz, 2H), 7.17-7.12 (m, 3H), 6.99 (dd, J=3.4, 6.0 Hz, 2H), 6.88 (d, J=8.9 Hz, 2H), 6.05 (d, J=1.7 Hz, 1H), 5.99 (d, J=1.8 Hz, 1H), 4.66 (d, J=10.4 Hz, 1H), 3.97 (d, J=12.7 Hz, 1H), 3.78 (d, J=1.2 Hz, 6H), 3.44 (s, 3H), 2.82 (dd, J=10.3, 12.7 Hz, 1H).

SFC: (Retention time: 3.73; 100% ee).

Optical rotation: (33.140±3.37°; c=0.5 g/100 mL diluted with methanol, 20° C.).

### Objective 4: Synthesis of (1S,2S,3R,3aS,8bR)-1,8b-dihydroxy-3a-(4-hydroxyphenyl)-6,8-dimethoxy-3-phenyl-2,3-dihydro-1H-cyclopenta[b]benzofuran-2-carboxamide (Compound 8) and (1R,2R,3S,3aR,8bS)-1,8b-dihydroxy-3a-(4-hydroxyphenyl)-6,8-dimethoxy-3-phenyl-2,3-dihydro-1H-cyclopenta[b]benzofuran-2-carboxamide (Compound 9)

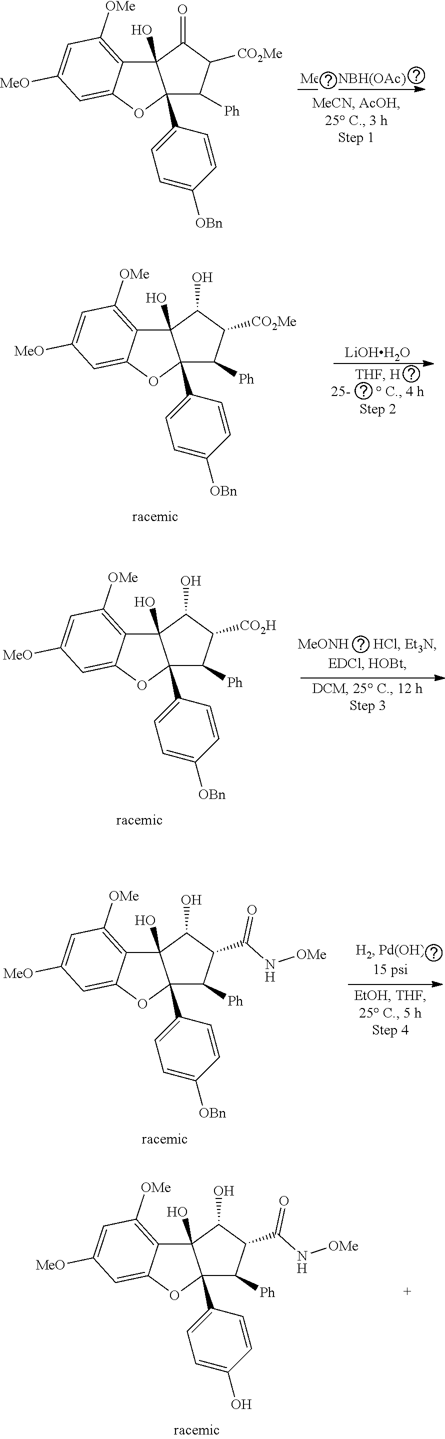

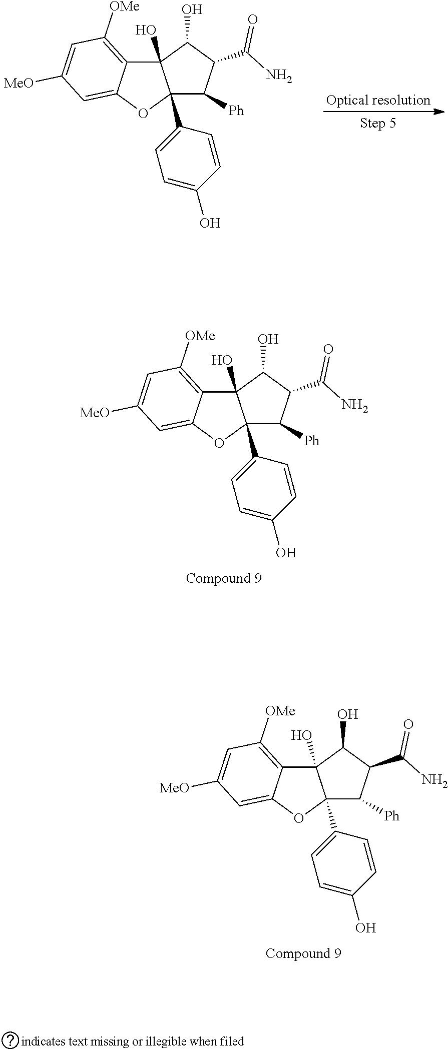 [SMILES: COc1cc(OC)c2c(c1)O[C@@]1(c3ccc(O)cc3)[C@H](c3ccccc3)[C@@H](C(N)=O)[C@@H](O)[C@@]21O ; COc1cc(OC)c2c(c1)O[C@@]1(c3ccc(O)cc3)[C@H](c3ccccc3)[C@@H](C(N)=O)[C@@H](O)[C@@]21O ; COc1cc(OC)c2c(c1)O[C@]1(c3ccc(O)cc3)[C@@H](c3ccccc3)[C@H](C(N)=O)[C@H](O)[C@]21O]

### Step 1: Methyl (1R,3aR,8bS)-3a-(4-benzyloxyphenyl)-1,8b-dihydroxy-6,8-dimethoxy-3-phenyl-2,3-dihydro-1H-cyclopenta[b]benzofuran-2-carboxylate

To a stirred solution of methyl (3aR,8bR)-3a-(4-benzyloxyphenyl)-8b-hydroxy-6,8-dimethoxy-1-oxo-3-phenyl-2,3-dihydrocyclopenta[b]benzofuran-2-carboxylate (2.6 g, 4.59 mmol, 1 eq) and AcOH (2.76 g, 45.89 mmol, 2.62 mL, 10 eq) in MeCN (30 mL) was added tetramethylammonium triacetoxyborohydride (7.24 g, 27.53 mmol, 6 eq). Then the mixture was stirred at 25° C. for 3 h, and then was poured into ice-water (50 mL). The aqueous phase was extracted with ethyl acetate (20 mL*4). The combined organic phases were washed with brine (20 mL), dried over anhydrous Na2SO4, filtered and concentrated in vacuum to give methyl (1R,3aR,8bS)-3a-(4-benzyloxyphenyl)-1,8b-dihydroxy-6,8-dimethoxy-3-phenyl-2,3-dihydro-1H-cyclopenta[b]benzofuran-2-carboxylate (2.6 g, crude) as light yellow solid. The crude product was used directly in next step without further purification.

LCMS: (M-17): 551.1 and (M+Na+): 591.1@1.277 min (5-95% ACN in H2O, 2.0 min).

### Step 2: (1R,3aR,8bS)-3a-(4-benzyloxyphenyl)-1,8b-dihydroxy-6,8-dimethoxy-3-phenyl-2,3-dihydro-1H-cyclopenta[b]benzofuran-2-carboxylic acid

To a solution of the methyl (1R,3aR,8bS)-3a-(4-benzyloxyphenyl)-1,8b-dihydroxy-6,8-dimethoxy-3-phenyl-2,3-dihydro-1H-cyclopenta[b]benzofuran-2-carboxylate (2.6 g, 4.57 mmol, 1 eq) in THF (28 mL) and H2O (7 mL) was added LiOH·H2O (767.00 mg, 18.28 mmol, 4.00 eq) at 25° C. The solution was stirred at 60° C. for 12 hours. The reaction mixture was diluted with water (50 mL). The solution was adjusted to pH=3 with aq HCl (2 N). Then the result solution was extracted with EtOAc (20 mL*5). The combined organic layer was washed with brine (40 mL), dried over Na2SO4, filtered and the filtrate was concentrated under reduced pressure to afford (1R,3aR,8bS)-3a-(4-benzyloxyphenyl)-1,8b-dihydroxy-6,8-dimethoxy-3-phenyl-2,3-dihydro-1H-cyclopenta[b]benzofuran-2-carboxylic acid (2.5 g, crude) as light yellow solid. The crude product was used directly in next step without further purification.

LCMS: (M-17): 537.4 @1.216 min (5-95% ACN in H2O, 2.0 min).

### Step 3: (1R,3aR,8bS)-3a-(4-benzyloxyphenyl)-1,8b-dihydroxy-N,6,8-trimethoxy-3-phenyl-2,3-dihydro-1H-cyclopenta[b]benzofuran-2-carboxamide

To a solution of (1R,3aR,8bS)-3a-(4-benzyloxyphenyl)-1,8b-dihydroxy-6,8-dimethoxy-3-phenyl-2,3-dihydro-1H-cyclopenta[b]benzofuran-2-carboxylic acid (2.5 g, 4.51 mmol, 1 eq), O-methylhydroxylamine hydrochloride (1.13 g, 13.51 mmol, 1.03 mL, 3.00 eq) in DCM (30 mL) was added HOBt (914.29 mg, 6.77 mmol, 1.5 eq), EDCI (1.04 g, 5.40 mmol, 1.2 eq) and TEA (2.05 g, 20.26 mmol, 2.82 mL, 4.49 eq) at 25° C. under nitrogen. Then the mixture was stirred at 25° C. for 12 h. The reaction mixture was diluted with water (50 mL). The solution was extracted with DCM (20 mL*4). The organic layer was washed with brine (30 mL), dried over Na2SO4, filtered and the filtrate was concentrated under reduced pressure. The residue was purified by column chromatography (SiO2, Petroleum ether/Ethyl acetate=1/1 to 1/4) to give (1R,3aR,8bS)-3a-(4-benzyloxyphenyl)-1,8b-dihydroxy-N,6,8-trimethoxy-3-phenyl-2,3-dihydro-1H-cyclopenta[b]benzofuran-2-carboxamide (1.9 g, 3.21 mmol, 71.29% yield, 98.72% purity) as white solid.

LCMS: (M+H+): 584.3 @1.153 min (5-95% ACN in H2O, 2.0 min).

HPLC: @3.532 min (10-80% ACN in H2O, 5.2 min).

Chiral SFC: Retention time: (Peak 1:3.50; Peak 2: 3.67).

### Step 4: 1,8b-dihydroxy-3a-(4-hydroxyphenyl)-N,6,8-trimethoxy-3-phenyl-2,3-dihydro-1H-cyclopenta[b]benzofuran-2-carboxamide

To a solution of (1R,3aR,8bS)-3a-(4-benzyloxyphenyl)-1,8b-dihydroxy-N,6,8-trimethoxy-3-phenyl-2,3-dihydro-1H-cyclopenta[b]benzofuran-2-carboxamide (0.6 g, 1.03 mmol, 1 eq) in EtOH (5 mL) and THF (5 mL) was added Pd(OH)2/C (1.03 mmol, 20% purity) under N2. The suspension was degassed under vacuum and purged with H2 three times. The mixture was stirred under H2 (15 psi) at 25° C. for 5 h. The reaction mixture was filtered through a pad of Celite and the filter cake was washed with THF (10 mL*4) and EtOH (10 mL*4). The filtrate was concentrated under reduced pressure to afford the crude product. The residue was purified by prep-TLC (Methanol/Ethyl acetate=20/1) to afford the two products (product 1: 180 mg; product 2: 280 mg). Each product was further purified by neutral prep-HPLC (product 1:column: Agela Durashell C18 150*25 5 u; mobile phase: [water (10 mM NH4HCO3)-ACN]; B %: 25%-%, 10 min; product 2: column: Agela Durashell C18 150*25 5 u; mobile phase: [water (10 mM NH4HCO3)-ACN]; B %: 23%-53%, 10 min) to afford the purified products.

### Product 1: 1,8b-dihydroxy-3a-(4-hydroxyphenyl)-N,6,8-trimethoxy-3-phenyl-2,3-dihydro-1H-cyclopenta[b]benzofuran-2-carboxamide

(130 mg crude, 15 mg pure) as white solid.

LCMS: (M+H+): 494.1 @2.313 min (10-80% ACN in H2O, 4.5 min).

1H NMR: (400 MHz, DMSO-d6) δ 11.12 (br s, 1H), 9.03 (s, 1H), 7.08-7.02 (m, 2H), 7.01-6.95 (m, 1H), 6.92-6.85 (m, 4H), 6.42 (d, J=8.7 Hz, 2H), 6.27 (d, J=1.7 Hz, 1H), 6.12 (d, J=1.7 Hz, 1H), 4.96 (s, 1H), 4.62 (d, J=3.3 Hz, 1H), 4.56 (br s, 1H), 4.14 (d, J=14.2 Hz, 1H), 3.78 (s, 3H), 3.75 (s, 3H), 3.55 (dd, J=5.5, 14.2 Hz, 1H), 3.49 (s, 3H).

### Product 2: 1,8b-dihydroxy-3a-(4-hydroxyphenyl)-6,8-dimethoxy-3-phenyl-2,3-dihydro-1H-cyclopenta[b]benzofuran-2-carboxamide

(115.3 mg, 248.77 μmol, 24.20% yield, 100% purity) as white solid.

LCMS: (M+H+): 464.1 @2.235 min (10-80% ACN in H2O, 4.5 min).

1H NMR: (400 MHz, DMSO-d6) δ 8.98 (s, 1H), 7.63 (br s, 1H), 7.07-7.00 (m, 2H), 6.99-6.88 (m, 5H), 6.40 (d, J=8.4 Hz, 2H), 6.24 (s, 1H), 6.09 (s, 1H), 4.88 (s, 1H), 4.61 (d, J=3.1 Hz, 1H), 4.57-4.52 (m, 1H), 4.11 (d, J=14.1 Hz, 1H), 3.77 (s, 3H), 3.73 (s, 3H).

### Step 5: (1R,2R,3S,3aR,8bS)-1,8b-dihydroxy-3a-(4-hydroxyphenyl)-6,8-dimethoxy-3-phenyl-2,3-dihydro-1H-cyclopenta[b]benzofuran-2-carboxamide (Compound 8) and (1S,2S,3R,3aS,8bR)-1,8b-dihydroxy-3a-(4-hydroxyphenyl)-6,8-dimethoxy-3-phenyl-2,3-dihydro-1H-cyclopenta[b]benzofuran-2-carboxamide (Compound 9)

Separation of the enantiomers of Product 2 from the previous step: Step 5a: Chiral SFC (Instrument: Thar SFC80 preparative SFC; Column: Chiralcel OJ-H 250*30 mm i.d. 5 u; Mobile phase: A for CO2 and B for MeOH (0.1% NH3H2O); Gradient: B %=30%; Flow rate: 65 g/min; Wavelength: 220 nm; Column temperature: 40° C.; System back pressure: 100 bar) gave compound 9 (Rt: 2.766 min, 18 mg), partially separated compound 8 (Rt: 3.040 min, 15 mg), and 20 mg mixture of compound 8 and compound 9.

Step 5b: Partially separated compound 8 from step 5a was further separated by SFC (Instrument: Thar SFC80 preparative SFC; Column: Chiralpak IC-H 250*30 mm i.d. 5 u; Mobile phase: A for C02 and B for EtOH (0.1% NH3H2O); Gradient: B %=42%; Flow rate: 70 g/min; Wavelength: 220 nm; Column temperature: 40° C.; System back pressure: 100 bar) again to give Compound 9 (Rt: 2.766 min, 2 mg), and Compound 8 (Rt: 3.040 min, 7 mg).

Step 5c: The 20 mg mixture of Compound 9 and Compound 8 from step 5a was separated by SFC (Instrument: Thar SFC80 preparative SFC; Column: Chiralpak IC-H 250*30 mm i.d. 5 u; Mobile phase: A for C02 and B for EtOH (0.1% NH3H2O); Gradient: B %=42%; Flow rate: 70 g/min; Wavelength: 220 nm; Column temperature: 40° C.; System back pressure: 100 bar) again to give purified compound 9 (Rt: 2.766 min, 6.5 mg) and compound 8 (Rt: 3.040 min, 3.1 mg).

A total of 26.5 mg of Compound 9 and 25.1 mg Compound 8 was obtained.

### Compound 8: (1R,2R,3S,3aR,8bS)-1,8b-dihydroxy-3a-(4-hydroxyphenyl)-6,8-dimethoxy-3-phenyl-2,3-dihydro-1H-cyclopenta[b]benzofuran-2-carboxamide

(25.1 mg, 54.16 μmol, 27.9% yield) was obtained as white solid.

LCMS: (M+H+): 464.0 @ 2.215 min (10-80% ACN in H2O, 4.5 min).

1H NMR: (400 MHz, ACETONITRILE-d3) δ 7.14-6.98 (m, 8H), 6.84 (s, 1H), 6.53 (d, J=8.7 Hz, 2H), 6.47 (br s, 1H), 6.26 (d, J=1.8 Hz, 1H), 6.16 (d, J=1.8 Hz, 1H), 5.79 (br s, 1H), 4.68 (d, J=4.0 Hz, 1H), 4.21 (d, J=14.2 Hz, 1H), 3.88-3.79 (m, 9H), 3.05 (s, 1H).

Chiral SFC: (Retention time: 3.04; 100% ee).

Optical rotation: (−71.88°±13.08°; c=0.5 g/100 mL diluted with methanol, 20° C.).

### Compound 9: (1S,2S,3R,3aS,8bR)-1,8b-dihydroxy-3a-(4-hydroxyphenyl)-6,8-dimethoxy-3-phenyl-2,3-dihydro-1H-cyclopenta[b]benzofuran-2-carboxamide

(26.5 mg, 57.2 μmol, 27.9% yield) was obtained as white solid.

LCMS: (M+H+): 464.0 @2.215 min (10-80% ACN in H2O, 4.5 min).

1H NMR: (400 MHz, ACETONITRILE-d3) δ7.14-7.00 (m, 8H), 6.84 (br s, 1H), 6.53 (d, J=8.7 Hz, 2H), 6.47 (br s, 1H), 6.26 (d, J=1.8 Hz, 1H), 6.17 (d, J=2.0 Hz, 1H), 5.78 (br s, 1H), 4.69 (d, J=5.6 Hz, 1H), 4.21 (d, J=14.2 Hz, 1H), 3.89-3.78 (m, 9H), 3.06 (br s, 1H).

Chiral SFC: (Retention time: 2.766; 98.6% ee).

Optical rotation: (66.27°±8.51°; 0.5 g/100 mL diluted with methanol, 20° C.).

### Summary of Compounds 1-9.

Compounds 1-9 are summarized in Table 1 above.

### Objective 5: Evaluation of Biological Activity In Vitro

### Examples 1-6: Objective and Methods

The following objective and methods relate to Examples 1-5.

### Objective:

Comparison of broad-spectrum antiviral activities of various synthetic rocaglates, and related biological activities.

### Materials & Methods:

Cryopreserved normal human bronchial epithelial (NHBE) cells were obtained from LONZA™. The undifferentiated cells were seeded on collagen IV-coated transwell plates (CORNING COSTAR™) and grown in a mixture of DMEM (INVITROGEN™) and BEGM (LONZA™) supplemented with retinoic acid (75 nM). Every other day, fresh medium was added and, after reaching confluence, the cells were cultivated under air-liquid conditions for at least four additional weeks to differentiate into a pseudostratified human airway epithelium. During this period, medium from the basolateral compartment was renewed every 2-3 days and the apical surface was washed once a week with PBS (INVITROGEN™).

Reagents. Compounds 1-9 were obtained as described above (see also WO 2019/161345, published 22 Aug. 2019 [PCT/US2019/018441, filed 18 Feb. 2019]) and were dissolved in DMSO for a total concentration of 10 mM (individual enantiomer or total racemic mixture) and stored at −20° C.

Primers. Primers utilized in various examples are found in Table 2.

TABLE 2
Primer sequence used for PCR-based site-
directed mutagenesis of pFR_HCV_xb
constructs.

| Con- | Primer_fwd | Primer_rev |
|---|---|---|
| struct | (5′-3′) | (5′-3′) |
| (AG)15 | AGAGAGAGAGAGAG | TTAAGCGGGTCGCT |
| | AGAGAGAGAGAGAG | GCAGG |
| | AGATGGAAGACGCC | (SEQ ID NO: 2) |
| | AAAAACATAAAGAA | |
| | AGG | |
| | (SEQ ID NO: 1) | |
| ß- | TCACTAGCAACCTC | ACACAGTTGTGTCA |
| Globin | AAACAGACACCATG | GAAGCAAATGTTTA |
| | GAAGACGCCAAAAA | AGCGGGTCGC |
| | C | (SEQ ID NO: 4) |
| | (SEQ ID NO: 3) | |

Human airway epithelial cells. Cryopreserved normal human bronchial epithelial (NHBE) cells were obtained from LONZA™. Undifferentiated cells were seeded on transwell plates (CORNING COSTAR™) coated with Collagen IV (INVITROGEN™) and grown in a mixture of DMEM (INVITROGEN™) and BEGM (LONZA™) supplemented with retinoic acid (75 nM). Fresh medium was added regularly after 2 days. After reaching confluence, the cells were cultivated under air-liquid conditions for 4 additional weeks for full differentiation into pseudostratified human airway epithelia. Medium from the basolateral compartment was renewed every 2-3 days, and the apical surface was washed every week with PBS (INVITROGEN™).

Antiviral activity. To calculate effective concentration 50% (half-maximal effective concentration; EC₅₀) values, the virus titer determined for virus-infected cells treated with DMSO only was set to 100%, and titers obtained treated cells were calculated in relation to it. EC₅₀ values were calculated by non-linear regression analysis using GRAPHPAD PRISM™ 6.0 (GRAPHPAD SOFTWARE™).

For the infection of the primary human airway epithelial cells, the apical surface was washed 3 times with PBS before the cells were infected with SARS-CoV-2 (MOI=3). After 1 h the inoculum was removed and the medium in the basal compartment was replaced with medium containing the indicated inhibitor concentrations. At the indicated time points, the apical surface of the cells was incubated with PBS for 15 min, and virus titers in the supernatants were determined by virus plaque assay.

Dual luciferase constructs. The constructs are based on the commercially available plasmid pFR_HCV_xb (ADDGENE™) and were produced using PCR-based site-directed mutagenesis. Primers were designed using SNAPGENE 4.1.9™ (GSL BIOTECH LLC™). Primer sequences are shown in Table 2. The respective 5′-UTRs were cloned downstream of the HSV-TK promotor directly followed by the firefly luciferase gene, an HCV IRES, and the Renilla luciferase gene.

Dualluciferase reporter assay. The dualluciferase reporter assay was done as described previously (Müller et al. [2018a] Antivir. Res. 150: 123-129). All experiments were performed in at least three independent replicates.

### Example 1: Analyses of the 5′-UTR-Mediated Inhibitory Activities of Synthetic Rocaglates

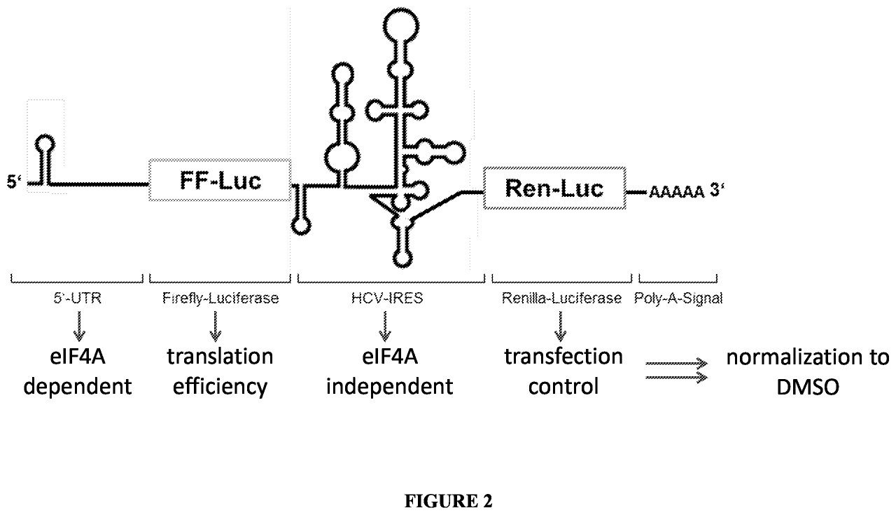

To gain more mechanistic insights regarding the effects of the synthetic rocaglates on translation initiation and RNA clamping, the inhibitory effects of the two rocaglates on different viral 5′-UTRs in a dual luciferase reporter assay were compared (FIG. 2; Müller et al. [2018a] Antivir. Res. 150: 123-129).

The dual luciferase assay was performed in human liver carcinoma HepG2 cells using the DUAL-LUCIFERASE® Reporter Assay System (PROMEGA™). The day before transfection, 2×10⁴ cells were seeded in a 96-well plate (CELLSTAR® 96-well Microplate, flat bottom black polystyrene wells, GREINER BIO-ONE™). 5-6 hours post transfection, fresh medium containing the synthetic rocaglate compounds or DMSO was added to the cells, followed by an incubation at 37° C. and 5% CO₂ for 48 h. The dual luciferase assay was performed according to the manufacturer's instructions in at least three independent replicates. The bioluminescence was measured using a SAFIRE™ II microplate reader (TECAN™). The constructs used for transfection are based on the plasmid pFR_HCV_xb (ADDGENE™) containing a firefly luciferase and a Renilla luciferase reporter gene, the latter regulated by an HCV-IRES. The respective 5′-UTRs were cloned upstream of the firefly luciferase gene using PCR based site-directed mutagenesis. As a negative control the 5′-UTR of the housekeeping gene ß-globin was used; as a positive control a 30 bp polypurine sequence ((AG)₁₅) was used. For analysis the firefly luciferase activities were normalized to the corresponding Renilla luciferase activities, followed by a normalization of the treated samples to the corresponding DMSO control.

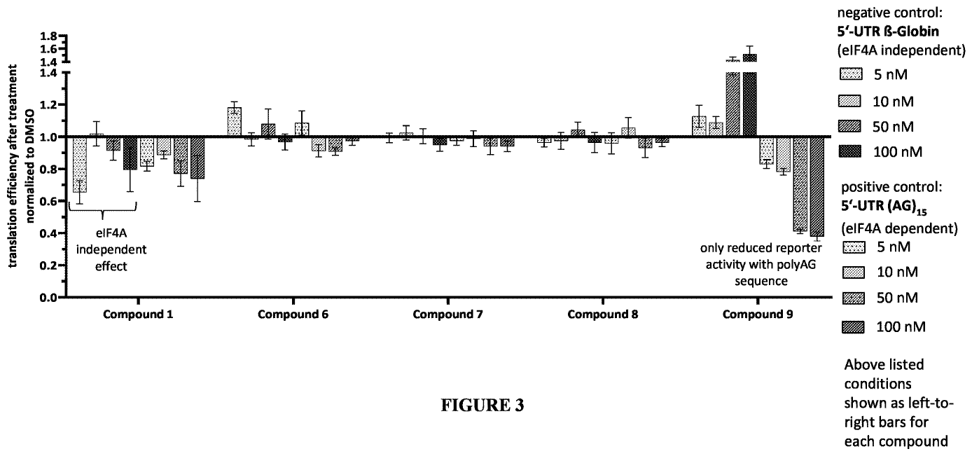

FIG. 3 is a graph showing the resulting analysis of the sensitivity of the 5′-UTR of beta-globin (β-globin; shades of blue) negative control (eIF4A-independent translation), and the sensitivity of the 5′-UTR of (AG)₁₅ ([AG]₁₅; shades of red) positive control (eIF4A-dependent translation), against 5, 10, 50, and 100 nM treatment with five rocaglate samples (Compound 1, Compound 6, Compound 7, Compound 8, Compound 9) in a dual luciferase assay. The reporter gene expression data were normalized to the transfection efficiencies and the corresponding DMSO controls. An eIF4A-independent effect was observed for Compound 1, while Compound 9 only reduced reporter activity with polyAG sequences. Therefore, only Compound 9 shows eIF4A-dependency, while Compound 1 has nonspecific effects (B-Globin 5′-UTR). Standard errors of the mean of at least three independent experiments are shown. MFE=minimal free energy (kcal/mol).

### Example 2: Further Studies of the Complexing of the Synthetic Rocaglates with the eIF4A Complex

A thermal shift assay was performed to study the complexing of the synthetic rocaglates with the eIF4A complex. For the thermal shift assay a final protein concentration of 5 μM purified recombinant human eIF4A (purification according to Iwasaki, et al., 2019) was used. In a reaction volume of 20 μL a 10-fold excess of polypurine RNA ((AG)5) (biomers) (final concentration: 50 μM), 1 mM of the ATP analogue AMP-PNP (ROCHE™), 100 μM of the synthetic rocaglate compounds or DMSO as well as 0.3 μL SYPRO® Orange Protein Gel Stain (SIGMA-ALDRICH™) were mixed with the protein. The measurement was performed in a MICROAMP® Fast 96-Well Reaction Plate (0.1 mL, APPLIED BIOSYSTEMS™) using the QUANTSTUDIO3™ Real-Time PCR System (APPLIED BIOSYSTEMS™). The melt curve starting temperature was set to 10° C. in increments of 0.05° C./s ending at 95° C. After measurement, the data was analyzed using the PROTEIN THERMAL SHIFT™ Software (APPLIED BIOSYSTEMS™).

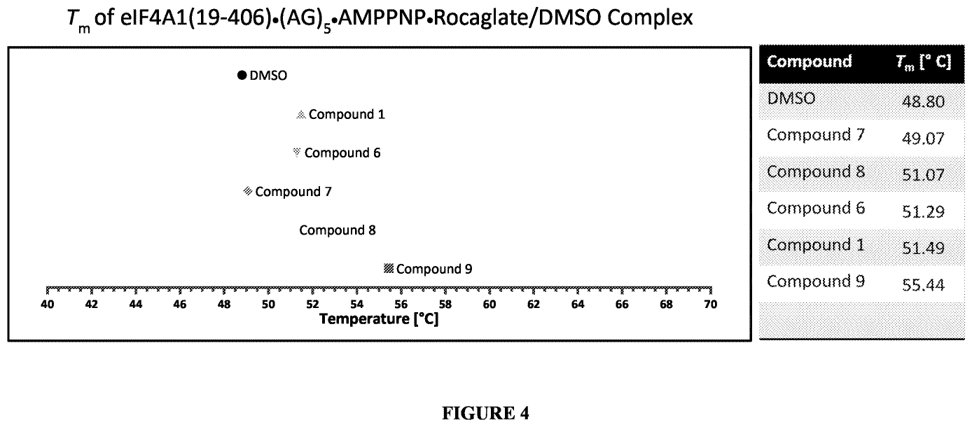

FIG. 4 shows a graph (left) and a table (right) depicting the melting temperature (Tm; Tm) of eIF4A1(19-406)-(AG)5-AMPPNP-Rocaglate/DMSO complexes, using DMSO as a negative control (Tm=48.80), as indicative of eIF4A binding of various rocaglate compounds (Compound 7 [Tm=49.07], Compound 8 [Tm=51.07], Compound 6 [Tm=51.29], Compound 1 [Tm=51.49], Compound 9 [Tm=55.44]), described herein (eIF4A1(19-406)=amino acid residues 19-406 of eIF4A1); AMPPNP=adenylyl-imidodiphosphate [AMP-PNP]).

### Example 3: Antiviral Activity of Synthetic Rocaglates

Cryopreserved normal human bronchial epithelial (NHBE) cells were obtained from LONZA™. The undifferentiated cells were seeded on collagen IV-coated transwell plates (CORNING COSTAR™) and grown in a mixture of DMEM (INVITROGEN™) and BEGM (LONZA™) supplemented with retinoic acid (75 nM). Every other day, fresh medium was added and, after reaching confluence, the cells were cultivated under air-liquid conditions for at least four additional weeks to differentiate into a pseudostratified human airway epithelium. During this period, medium from the basolateral compartment was renewed every 2-3 days and the apical surface was washed once a week with PBS (INVITROGEN™).

For the infection of NHBE cells, the apical surface was washed 3 times with PBS and cells were infected with SARS-CoV-2 (MOI=3). After 1 h, the inoculum was removed and the medium in the basal compartment was replaced with medium containing the indicated inhibitor concentration. At the indicated time points p.i., the apical surface of the cells was incubated with PBS for 15 min and virus titers in the supernatants were determined by plaque assay. Briefly, cells were seeded in 24-well plates and inoculated for 1 h with 10-fold virus dilutions in PBS/BA/P/S. Next, the virus inoculum was replaced with Avicel-containing medium (1×MEM [GIBCO™], 1.25% Avicel [FMC BIOPOLYMER™]). At 24 and 48 h post infection (p.i.), the plates were washed with PBS, fixed with 3.7% PFA in PBS and the cell layer was stained with 0.15% crystal violet. The virus titer determined for DMSO treated virus-infected controls was set to 100%.

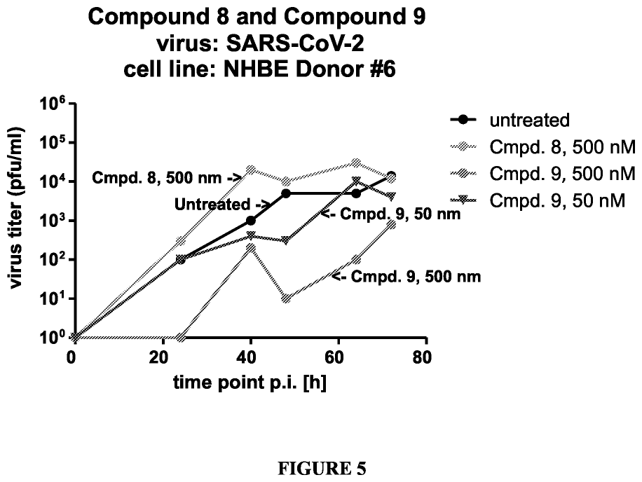

FIG. 5 is a graph depicting the comparative antiviral effects of Compound 8, Compound 9, and untreated cells (bottom) in Normal Human Bronchial Epithelial cells (NHBE cells), as a measure of virus titer over time, from a donor whose cells were infected with SARS-CoV-2 (COVID-19 virus). The cells of the donor were treated with 50 nM or 500 nM Compound 9 as compared with untreated cells or with cells treated with 500 nM Compound 8 as controls.

### Example 4: Imaging of Cells Treated with Synthetic Rocaglates

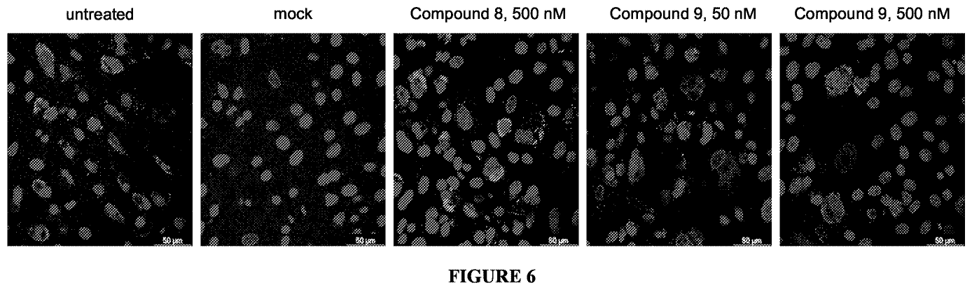

FIG. 6 is an immunofluorescence analysis to determine the effects of Compound 8 and 9 on viral dsRNA accumulation in SARS-CoV-2 infected Vero E6 cells. After infection and treatment with Compound 8 and 9 cells were fixed at 24 h p.i. and analyzed by confocal microscopy using a mouse anti-dsRNA mAB (J2, SCICONS English & Scientific Consulting Kft) that detects viral dsRNA replication intermediates (red) (Müller et al., 2018). Cell nuclei were stained with 2-(4-amidinophenyl)-1H-indole-6-carboxamidine (4′,6-diamidino-2-phenylindole; DAPI; blue). Samples were treated as follows: untreated control, mock infection control, Compound 8 (500 nM), Compound 9 (50 nM), Compound 9 (500 nM) (left to right).

WO 2019/161345 (published 22 Aug. 2019; PCT/US2019/018441, filed 18 Feb. 2019) and all other references cited herein are hereby incorporated by reference.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating a viral infection in a host cell or organism infected by a virus, the method comprising administering to the cell or organism a therapeutically effective amount of a pharmaceutical composition comprising a synthetic rocaglate composition or a pharmaceutically acceptable salt thereof, wherein the synthetic rocaglate composition comprises a compound represented by formula (I) 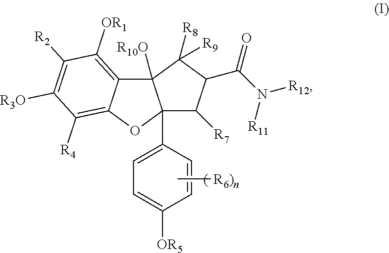
wherein
R1, R3, and R5 are each independently H, alkyl, —P(O)(OH)(OH), —CH2-P(O)(OH)(OH), —P(O)(OH)(O-alkyl), —CH2-P(O)(OH)(O-alkyl), —P(O)(O-alkyl)(O-alkyl), —CH2-P(O)(O-alkyl)(O-alkyl), or (CO)-alkyl, or a pharmaceutically acceptable salt of —P(O)(OH)(OH), —CH2-P(O)(OH)(OH), —P(O)(OH)(O-alkyl), or —CH2-P(O)(OH)(O-alkyl), wherein at least one of R1, R3, and R5 is H, —P(O)(OH)(OH), —CH2-P(O)(OH)(OH), —P(O)(OH)(O-alkyl), —CH2-P(O)(OH)(O-alkyl), —P(O)(O-alkyl)(O-alkyl), —CH2-P(O)(O-alkyl)(O-alkyl), or a pharmaceutically acceptable salt of —P(O)(OH)(OH), —CH2-P(O)(OH)(OH), —P(O)(OH)(O-alkyl), or —CH2-P(O)(OH)(O-alkyl);
R2 and R4 are each independently H, alkyl, halo, nitro, OH, O-alkyl, SH, S-alkyl, CN, haloalkyl, O-haloalkyl, NRaRb, (CO)-alkyl, (CO)OH, (CO)O-alkyl, SO2NRaRb, (CO)NRaRb, NH(CO)-alkyl, cycloalkyl, heterocycloalkyl, aryl, or heteroaryl, wherein aryl and heteroaryl are optionally substituted with alkyl, halo, nitro, OH, O-alkyl, SH, S-alkyl, CN, haloalkyl, O-haloalkyl, and NRaRb;
R6 is alkyl, halo, nitro, OH, O-alkyl, SH, S-alkyl, CN, haloalkyl, O-haloalkyl, NRaRb, (CO)-alkyl, (CO)OH, (CO)O-alkyl, SO2NRaRb, (CO)NRaRb, NH(CO)-alkyl, cycloalkyl, heterocycloalkyl, aryl, or heteroaryl, wherein aryl and heteroaryl are optionally substituted with alkyl, halo, nitro, OH, O-alkyl, SH, S-alkyl, CN, haloalkyl, O-haloalkyl, and NRaRb;
R7 is aryl or heteroaryl, wherein aryl and heteroaryl are optionally substituted with alkyl, halo, nitro, OH, O-alkyl, SH, S-alkyl, CN, haloalkyl, O-haloalkyl, and NRaRb;
R8 and R9 are each independently H, OH, alkyl, halo, O-alkyl, SH, S-alkyl, CN, haloalkyl, O-haloalkyl, NRaRb, (CO)OH, (CO)O-alkyl, SO2NRaRb, (CO)NRaRb, or NH(CO)-alkyl;
R10 is H, alkyl, (CO)-alkyl, or (CO)NRaRb;
R11 and R12 are each independently H, OH, alkyloxy, cycloalkyloxy, heterocycloalkyloxy, cycloalkylalkyloxy, heterocycloalkylalkyloxy, arylalkyloxy, heteroarylalkyloxy, aryloxy, or heteroaryloxy;
Ra and Rb are each H or alkyl, or Ra and Rb, together with the nitrogen atom they are attached, form a heterocycloalkyl group; and
n is an integer from 0 to 4,
or a pharmaceutically acceptable salt thereof.

2. The method of claim 1, wherein the synthetic rocaglate composition comprises 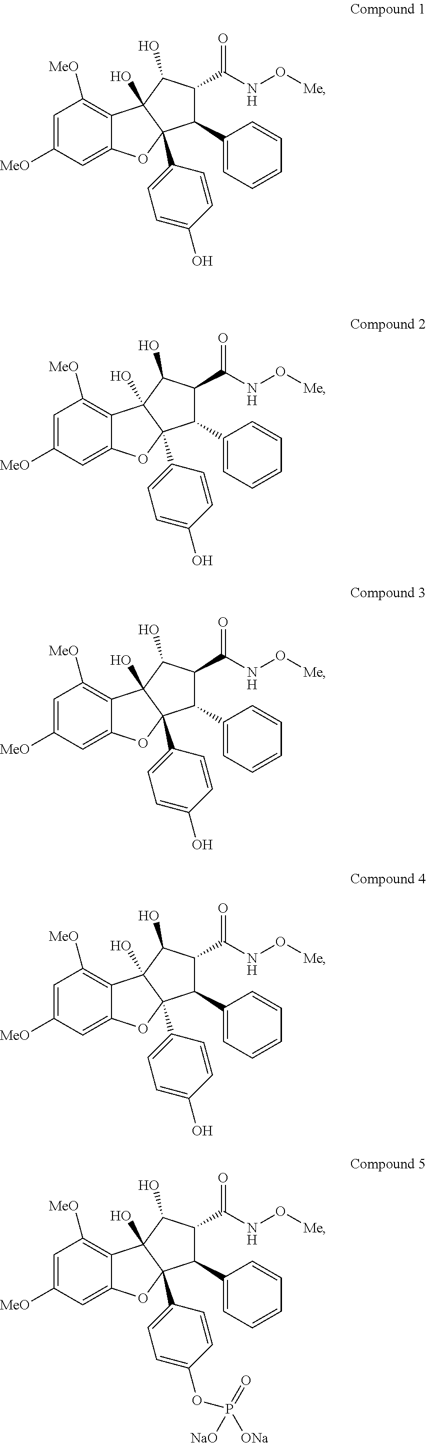 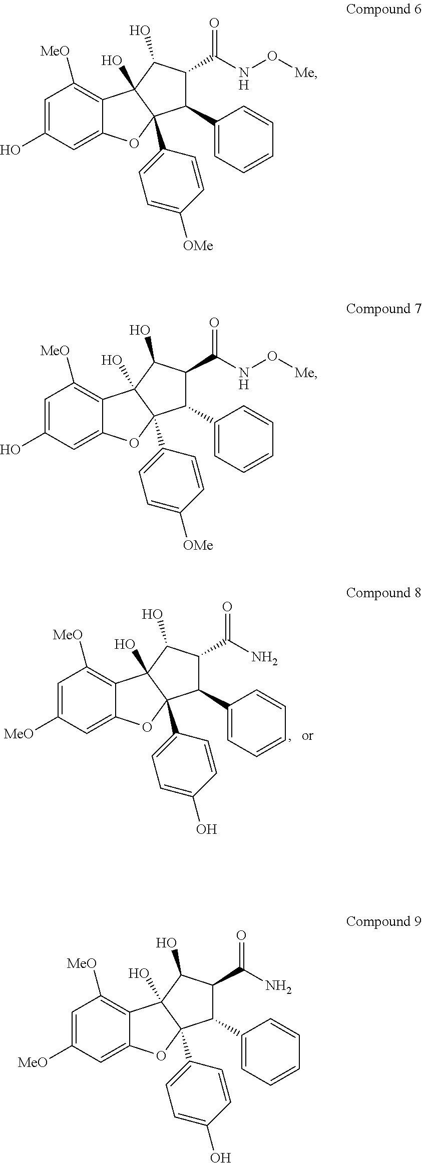 [SMILES: CONC(=O)[C@H]1[C@@H](O)[C@@]2(O)c3c(OC)cc(O)cc3O[C@@]2(c2ccc(OC)cc2)[C@@H]1c1ccccc1 ; CONC(=O)[C@@H]1[C@H](O)[C@]2(O)c3c(OC)cc(O)cc3O[C@]2(c2ccc(OC)cc2)[C@H]1c1ccccc1 ; COc1cc(OC)c2c(c1)O[C@@]1(c3ccc(O)cc3)[C@H](c3ccccc3)[C@@H](C(N)=O)[C@@H](O)[C@@]21O ; COc1cc(OC)c2c(c1)O[C@]1(c3ccc(O)cc3)[C@@H](c3ccccc3)[C@H](C(N)=O)[C@H](O)[C@]21O]
or an enantiomer of any thereof, a racemic mixture of any thereof, or a combination of any thereof.

3.-5. (canceled)

6. The method of claim 1, wherein the synthetic rocaglate composition reduces or inhibits a eukaryotic initiation factor 4A (eIF4A) activity, reduces or inhibits the eIF4A helicase activity, or reduces or inhibits eIF4A clamping to a 5′-untranslated region (5′-UTR) of the mRNA of the virus.

7.-8. (canceled)

9. The method of claim 6, wherein the 5′-UTR comprises a hairpin structure or a polypurine sequence element comprising at least 10 purine nucleotides.

10. (canceled)

11. The method of claim 9, wherein the polypurine sequence element comprises at least 20 purine nucleotides.

12. The method of claim 1, wherein the virus comprises a virus from the Coronaviridae family, the Arenaviridae family, the Nairoviridae family, the Flaviviridae family, the Hepeviridae family, the Filoviridae family, or the Togaviridae family.

13. The method of claim 12, wherein the virus from the Coronaviridae family comprises severe acute respiratory syndrome coronavirus 2 (SARS-CoV-2, COVID-19 virus), human coronavirus 229E (HCoV-229E), Middle East respiratory syndrome coronavirus (MERS-CoV), severe acute respiratory syndrome coronavirus (SARS-CoV), human coronavirus OC43 (HCoV-OC43), human coronavirus NL63 (HCoV-NL63), or human coronavirus HKU1 (HCoV-HKU1).

14. The method of claim 12, wherein the virus from the Arenaviridae family comprises Lassa mammarenavirus (LASV), Guanarito mammarenavirus, Junin mammarenavirus, Lujo mammarenavirus, Machupo mammarenavirus, Sabia mammarenavirus, or Whitewater Arroyo mammarenavirus.

15. The method of claim 12, wherein the virus from the Nairoviridae family comprises Crimean-Congo hemorrhagic fever virus (CCHFV).

16. The method of claim 12, wherein the virus from the Flaviviridae family comprises Zika virus (ZIKV), hepacivirus C (hepatitis C virus, HepC), dengue fever virus, yellow fever virus, Japanese encephalitis virus, or West Nile virus.

17. The method of claim 12, wherein the virus from the Hepeviridae family comprises hepatitis E virus (HEV) or hepatitis B virus.

18. The method of claim 12, wherein the virus from the Filoviridae family comprises Ebolavirus, Marburgvirus, Dianlovirus, Cuevavirus, Striavirus, or Thamnovirus.

19. The method of claim 12, wherein the virus from the Togaviridae family comprises an Alphavirus.

20. The method of claim 19, wherein the virus from the Alphavirus comprises Chikungunya virus, Eastern equine encephalitis virus, Western equine encephalitis virus, Barmah Forest virus, Mayaro virus, O'nyong'nyong virus, Ross river virus, Semliki Forest virus, Sindbis virus, Una virus, Tonate virus, or Venezuelan equine encephalitis.

21. The method of claim 1, wherein the pharmaceutical composition further comprises a pharmaceutically acceptable carrier, excipient, or diluent.

22. A method for reducing or inhibiting translation initiation of a messenger ribonucleic acid (mRNA) of a virus in a host cell or organism infected by the virus, the method comprising administering to the cell or organism a therapeutically effective amount of a pharmaceutical composition comprising a synthetic rocaglate composition or a pharmaceutically acceptable salt thereof, wherein the synthetic rocaglate composition comprises a compound represented by formula (I) 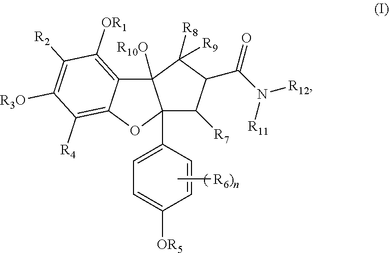
wherein
R1, R3, and R5 are each independently H, alkyl, —P(O)(OH)(OH), —CH2-P(O)(OH)(OH), —P(O)(OH)(O-alkyl), —CH2-P(O)(OH)(O-alkyl), —P(O)(O-alkyl)(O-alkyl), —CH2-P(O)(O-alkyl)(O-alkyl), or (CO)-alkyl, or a pharmaceutically acceptable salt of —P(O)(OH)(OH), —CH2-P(O)(OH)(OH), —P(O)(OH)(O-alkyl), or —CH2-P(O)(OH)(O-alkyl), wherein at least one of R1, R3, and R5 is H, —P(O)(OH)(OH), —CH2-P(O)(OH)(OH), —P(O)(OH)(O-alkyl), —CH2-P(O)(OH)(O-alkyl), —P(O)(O-alkyl)(O-alkyl), —CH2-P(O)(O-alkyl)(O-alkyl), or a pharmaceutically acceptable salt of —P(O)(OH)(OH), —CH2-P(O)(OH)(OH), —P(O)(OH)(O-alkyl), or —CH2-P(O)(OH)(O-alkyl);
R2 and R4 are each independently H, alkyl, halo, nitro, OH, O-alkyl, SH, S-alkyl, CN, haloalkyl, O-haloalkyl, NRaRb, (CO)-alkyl, (CO)OH, (CO)O-alkyl, SO2NRaRb, (CO)NRaRb, NH(CO)-alkyl, cycloalkyl, heterocycloalkyl, aryl, or heteroaryl, wherein aryl and heteroaryl are optionally substituted with alkyl, halo, nitro, OH, O-alkyl, SH, S-alkyl, CN, haloalkyl, O-haloalkyl, and NRaRb;
R6 is alkyl, halo, nitro, OH, O-alkyl, SH, S-alkyl, CN, haloalkyl, O-haloalkyl, NRaRb, (CO)-alkyl, (CO)OH, (CO)O-alkyl, SO2NRaRb, (CO)NRaRb, NH(CO)-alkyl, cycloalkyl, heterocycloalkyl, aryl, or heteroaryl, wherein aryl and heteroaryl are optionally substituted with alkyl, halo, nitro, OH, O-alkyl, SH, S-alkyl, CN, haloalkyl, O-haloalkyl, and NRaRb;
R7 is aryl or heteroaryl, wherein aryl and heteroaryl are optionally substituted with alkyl, halo, nitro, OH, O-alkyl, SH, S-alkyl, CN, haloalkyl, O-haloalkyl, and NRaRb;
R8 and R9 are each independently H, OH, alkyl, halo, O-alkyl, SH, S-alkyl, CN, haloalkyl, O-haloalkyl, NRaRb, (CO)OH, (CO)O-alkyl, SO2NRaRb, (CO)NRaRb, or NH(CO)-alkyl;
R10 is H, alkyl, (CO)-alkyl, or (CO)NRaRb;
R11 and R12 are each independently H, OH, alkyloxy, cycloalkyloxy, heterocycloalkyloxy, cycloalkylalkyloxy, heterocycloalkylalkyloxy, arylalkyloxy, heteroarylalkyloxy, aryloxy, or heteroaryloxy;
Ra and Rb are each H or alkyl, or Ra and Rb, together with the nitrogen atom they are attached, form a heterocycloalkyl group; and
n is an integer from 0 to 4,
or a pharmaceutically acceptable salt thereof.

23. The method of claim 22, wherein the synthetic rocaglate composition comprises 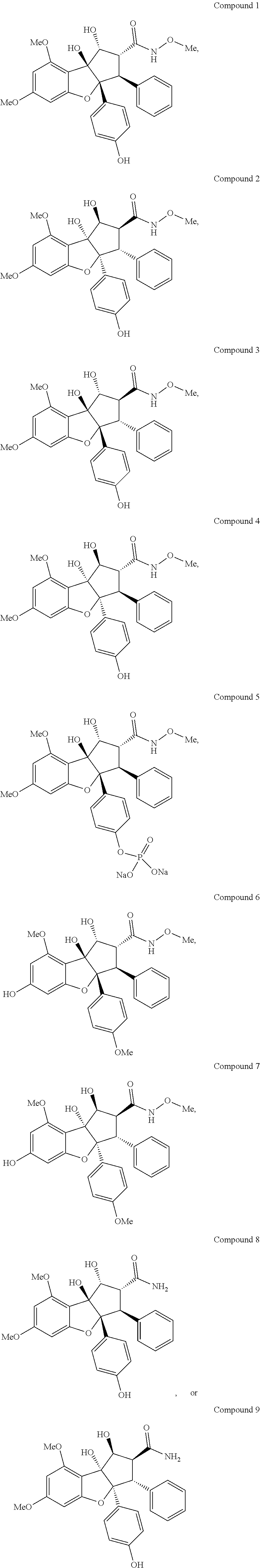
or an enantiomer of any thereof, a racemic mixture of any thereof, or a combination of any thereof.

24.-33. (canceled)

34. The method of claim 22, wherein the virus comprises a virus from the Coronaviridae family, the Arenaviridae family, the Nairoviridae family, the Flaviviridae family, the Hepeviridae family, the Filoviridae family, or the Togaviridae family.

35. The method of claim 34, wherein the virus from the Coronaviridae family comprises severe acute respiratory syndrome coronavirus 2 (SARS-CoV-2, COVID-19 virus), human coronavirus 229E (HCoV-229E), Middle East respiratory syndrome coronavirus (MERS-CoV), severe acute respiratory syndrome coronavirus (SARS-CoV), human coronavirus OC43 (HCoV-OC43), human coronavirus NL63 (HCoV-NL63), or human coronavirus HKU1 (HCoV-HKU1).

36.-59. (canceled)


SEQUENCE LISTING (4 sequences)

SEQ ID NO: 1 - DNA, 59 bp, Artificial Sequence
  Forward primer for (AG)15
agagagagagagagagagagagagagagagatggaagacgccaaaaacataaagaaagg

SEQ ID NO: 2 - DNA, 19 bp, Artificial Sequence
  Reverse primer for (AG)15
ttaagcgggtcgctgcagg

SEQ ID NO: 3 - DNA, 43 bp, Artificial Sequence
  Forward primer for beta-globin
tcactagcaacctcaaacagacaccatggaagacgccaaaaac

SEQ ID NO: 4 - DNA, 38 bp, Artificial Sequence
  Reverse primer for beta-globin
acacagttgtgtcagaagcaaatgtttaagcgggtcgc